SEQUENCE LISTING (46 sequences)

SEQ ID NO: 1 - protein, 19 aa, Artificial Sequence
  IgA1 hinge
VPSTPPTPSPSTPPTPSPS

SEQ ID NO: 2 - protein, 6 aa, Artificial Sequence
  IgA2 hinge
VPPPPP

SEQ ID NO: 3 - protein, 58 aa, Artificial Sequence
  IgD hinge
ESPKAQASSVPTAQPQAEGSLAKATTAPATTRNTGRGGEEKKKEKEKEEQEERETKTP

SEQ ID NO: 4 - protein, 15 aa, Artificial Sequence
  IgG1 hinge
EPKSCDKTKTCPPCP

SEQ ID NO: 5 - protein, 12 aa, Artificial Sequence
  IgG2 hinge
ERKCCVECPPCP

SEQ ID NO: 6 - protein, 17 aa, Artificial Sequence
  IgG3 hinge
ELKTPLGDTTHTCPRCP

SEQ ID NO: 7 - protein, 15 aa, Artificial Sequence
  IgG3 hinge
EPKSCDTPPPCPRCP

SEQ ID NO: 8 - protein, 12 aa, Artificial Sequence
  IgG4 hinge
ESKYGPPCPSCP

SEQ ID NO: 9 - protein, 34 aa, Artificial Sequence
  IgG1 hinge_IgG1CH2
EPKSCDKTHTCPPCPAPELLGGPSTPPTPSPSGG

SEQ ID NO: 10 - protein, 9 aa, Artificial Sequence
  N-terminus of human IgG1 CH2
APELLGGPS

SEQ ID NO: 11 - protein, 8 aa, Artificial Sequence
  human IgG1 hinge
TPPTPSPS

SEQ ID NO: 12 - protein, 32 aa, Artificial Sequence
  synthetic polypeptide
EPKSCDKTHTCPPCPAPELLPSTPPSPSTPGG

SEQ ID NO: 13 - protein, 5 aa, Artificial Sequence
  N-terminus of human IgG1 CH2
APELL

SEQ ID NO: 14 - protein, 10 aa, Artificial Sequence
  synthetic polypeptide
PSTPPSPSTP

SEQ ID NO: 15 - protein, 327 aa, Artificial Sequence
  mutIGHG4 S228P mutant of IgG4 isotype
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 330 aa, Artificial Sequence
  IGHG1*01-L234A/L235A|Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 8 aa, Artificial Sequence
  Heavy_K3_P4_CDHR1
GYTFTEYS

SEQ ID NO: 18 - protein, 8 aa, Artificial Sequence
  Heavy_K3_P4_CDHR2
ISPNSGGT

SEQ ID NO: 19 - protein, 11 aa, Artificial Sequence
  Heavy_K3_P4_CDHR3
ARRDFHSSFDY

SEQ ID NO: 20 - protein, 6 aa, Artificial Sequence
  Light_K3_P4_CDRL1
QSISDY

SEQ ID NO: 21 - protein, 3 aa, Artificial Sequence
  Light_K3_P4_CDRL2
YAS

SEQ ID NO: 22 - protein, 9 aa, Artificial Sequence
  Light_K3_P4_CDRL3
QNGHSFPLT

SEQ ID NO: 23 - protein, 118 aa, Artificial Sequence
  K3_P4_VH
EVQLQQSGPELVKPGTSVKISCKTSGYTFTEYSMHWVKQSHGKSLEWFGGISPNSGGTSYNQKFKGKATLTVDKSSSTAYMELRSLTSEDSAVYYCARRDFHSSFDYWGQGTTLTVSS

SEQ ID NO: 24 - protein, 107 aa, Artificial Sequence
  K3_P4_VL
DIVMTQSPATLSVTPGDRVSLSCRASQSISDYLHWFQQKSHESPRLLIKYASQSISGIPSRFSGSGSGSDFTLSINSVEPEDVGVYYCQNGHSFPLTFGPGTKLELK

SEQ ID NO: 25 - protein, 118 aa, Artificial Sequence
  K3_P4_humanized_VH
EVQLQQSGAEVKKPGSSVKVSCKTSGYTFTEYSMHWVRQAPGKSLEWFGGISPNSGGTSYNQKFKGRATLTVDKSSSTAYMELSSLRSEDTAVYYCARRDFHSSFDYWGQGTTVTVSS

SEQ ID NO: 26 - protein, 107 aa, Artificial Sequence
  K3_P4_humanized_VL
DIVMTQSPATLSLSPGERATLSCRASQSISDYLHWFQQKPHESPRLLIKYASQSISGIPARFSGSGSGSDFTLTISSLEPEDFAVYYCQNGHSFPLTFGPGTKVDIK

SEQ ID NO: 27 - protein, 118 aa, Artificial Sequence; identical to SEQ ID NO: 23
  K3b_VH

SEQ ID NO: 28 - protein, 107 aa, Artificial Sequence
  K3b_VL
DIVLTQSPATLSVTPGNSVSLSCRASQSISDYLHWFQQKSHESPRLLIKYASQSISGIPSRFSGSGSGSDFTLSINSVEPEDVGVYYCQNGHSFPLTFGPGTKLELK

SEQ ID NO: 29 - protein, 118 aa, Artificial Sequence; identical to SEQ ID NO: 25
  K3b_Humanized_VH

SEQ ID NO: 30 - protein, 107 aa, Artificial Sequence
  K3b_Humanized_VL
DIVLTQSPATLSLSPGERATLSCRASQSISDYLHWFQQKPHESPRLLIKYASQSISGIPARFSGSGSGSDFTLTISSLEPEDFAVYYCQNGHSFPLTFGPGTKVDIK

SEQ ID NO: 31 - protein, 233 aa, Artificial Sequence
  light chain 1 anti-NkP46
MEWSGVFMFLLSVTAGVHSDIVMTQSPATLSLSPGERATLSCRASQSISDYLHWFQQKPHESPRLLIKYASQSISGIPARFSGSGSGSDFTLTISSLEPEDFAVYYCQNGHSFPLTFGPGTKVDIKRTVAAPAVFIFPPSDEQLKSGTASVVCLLKNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 8 aa, Artificial Sequence
  Anti-CD38_VH1_CDRH1
GYTFTSYW

SEQ ID NO: 33 - protein, 8 aa, Artificial Sequence
  Anti-CD38_VH1_CDRH2
IYPGDGDT

SEQ ID NO: 34 - protein, 14 aa, Artificial Sequence
  Anti-CD38_VH1_CDRH3
ARERTTGAPRYFDV

SEQ ID NO: 35 - protein, 6 aa, Artificial Sequence
  Anti-CD38_VH1_CDRL1
ENIYSF

SEQ ID NO: 36 - protein, 3 aa, Artificial Sequence
  Anti-CD38_VH1_CDRL2
NTK

SEQ ID NO: 37 - protein, 9 aa, Artificial Sequence
  Anti-CD38_VH1_CDRL3
QHHYGIPLT

SEQ ID NO: 38 - protein, 121 aa, Artificial Sequence
  Anti-CD38_VH
QVQLVQSGAEVKKPGASVKVSCKASGYTFTSYWMQWVRQAPGQGLEWMGAIYPGDGDTRYTQKFKGRVTMTRDTSTSTVYMELSSLRSEDTAVYYCARERTTGAPRYFDVWGQGTLVTVSS

SEQ ID NO: 39 - protein, 107 aa, Artificial Sequence
  Anti-CD38_VL
DIQMTQSPSSLSASVGDRVTITCRASENIYSFLAWYQQKPGKAPKLLIYNTKTLTEGVPSRFSGSGSGTDFTLTISSLQPEDFATYYCQHHYGIPLTFGGGTKVEIK

SEQ ID NO: 40 - protein, 233 aa, Artificial Sequence
  light chain 2 anti-CD38
MEWSGVFMFLLSVTAGVHSDIQMTQSPSSLSASVGDRVTITCRASENIYSFLAWYQQKPGKAPKLLIYNTKTLTEGVPSRFSGSGSGTDFTLTISSLQPEDFATYYCQHHYGIPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 721 aa, Artificial Sequence
  Fused-Heavy chain CD38/NKp46/huIgG1-L234A/L235A
MEWSGVFMFLLSVTAGVHSQVQLVQSGAEVKKPGASVKVSCKASGYTFTSYWMQWVRQAPGQGLEWMGAIYPGDGDTRYTQKFKGRVTMTRDTSTSTVYMELSSLRSEDTAVYYCARERTTGAPRYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPGGGGSGGSGSGGEVQLQQSGAEVKKPGSSVKVSCKTSGYTFTEYSMHWVRQAPGKSLEWFGGISPNSGGTSYNQKFKGRATLTVDKSSSTAYMELSSLRSEDTAVYYCARRDFHSSFDYWGQGTTVTVSSASTKGPSVFPLAPSSKSTSGGTAALGCLVKDYFPEPVTVSWNSGALTSGVHTFPAVLQSSGLYSLSSVVEVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 326 aa, Artificial Sequence
  IGHG1*01 wildtype (human)
GPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 118 aa, Artificial Sequence
  K3_Humanized_VL
DIVLTQSPATLSLSPGERATLSCRASQSISDYLHWFQQKPHESPRLLIKYASQSISGIPARFSGSGSGSDFTLTISSLEPEDFAVYYCQNGHSFPLTFGPGTKVDIKRTVAAPSVFIF

SEQ ID NO: 44 - protein, 35 aa, Artificial Sequence
  synthetic polypeptide
EPKSCDKTHTCPPCPAPELLGGPGGGGSGGSGSGG

SEQ ID NO: 45 - protein, 21 aa, Artificial Sequence
  non-repetitive linker
SPNSASHSGSAPQTSSAPGSQ

SEQ ID NO: 46 - protein, 721 aa, Artificial Sequence
  Fused-Heavy chain CD38/NKp46/huIgG1 wt
MEWSGVFMFLLSVTAGVHSQVQLVQSGAEVKKPGASVKVSCKASGYTFTSYWMQWVRQAPGQGLEWMGAIYPGDGDTRYTQKFKGRVTMTRDTSTSTVYMELSSLRSEDTAVYYCARERTTGAPRYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPGGGGSGGSGSGGEVQLQQSGAEVKKPGSSVKVSCKTSGYTFTEYSMHWVRQAPGKSLEWFGGISPNSGGTSYNQKFKGRATLTVDKSSSTAYMELSSLRSEDTAVYYCARRDFHSSFDYWGQGTTVTVSSASTKGPSVFPLAPSSKSTSGGTAALGCLVKDYFPEPVTVSWNSGALTSGVHTFPAVLQSSGLYSLSSVVEVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2024/0199756 A1
(43) Pub. Date: Jun. 20, 2024

(54) BISPECIFIC ANTIBODIES TARGETING NKP46 AND CD38 AND METHODS OF USE THEREOF

(71) Applicant: CYTOVIA THERAPEUTICS, LLC., Aventura, FL (US)

(72) Inventors: Jean KADOUCHE, Aventura, FL (US); Wei LI, Aventura, FL (US); Daniel TEPER, Aventura, FL (US)

(73) Assignee: CYTOVIA THERAPEUTICS, LLC., Aventura, FL (US)

(21) Appl. No.: 18/285,633

(22) PCT Filed: Apr. 5, 2022
(86) PCT No.: PCT/US2022/023501

§ 371 Date: Oct. 4, 2023

Related U.S. Application Data
(60) Provisional application No. 63/170,917, filed on Apr. 5, 2021.

(51) Int. Cl. C07K 16/28; A61K 39/00; A61K 39/395; A61K 41/17; A61P 35/00

(52) U.S. Cl. CPC C07K 16/2896; A61K 39/3955; A61K 39/4613; A61K 41/17; A61P 35/00; C07K 16/2803; C07K 2317/21; C07K 2317/24; C07K 2317/31

(57) ABSTRACT

This disclosure provides bispecific antibody molecules that specifically bind to NKp46 and CD38. The disclosure further relates to combination therapies comprising the bispecific antibody molecules. The bispecific antibody molecules can be used to treat, prevent and/or diagnose cancer or infectious conditions or disorders associated with cells expressing NKp46 and/or CD38.

### CROSS REFERENCE TO RELATED APPLICATIONS

This application claims priority to and the benefit of U.S. Provisional Application No. 63/170,917, filed Apr. 5, 2021, which is incorporated herein by reference in its entirety.

### REFERENCE TO SEQUENCE LISTING SUBMITTED ELECTRONICALLY

The contents of the text filed named “CYTT-002_001WO_SeqListing_ST25.txt”, which was created on Apr. 5, 2022 and is 44,213 bytes in size, are hereby incorporated by reference in their entirety.

### BACKGROUND

Cancer immunotherapy is utilized for generating and augmenting an anti-tumor immune response, e.g., by treatment with antibodies specific to antigens on tumor cells, with fusions of antigen presenting cells with tumor cells, or by specific activation of anti-tumor NK cells or T cells. The ability of recruiting immune cells against tumor cells in a patient provides a therapeutic modality of fighting cancer types and metastasis that so far were considered incurable.

Lymphocytes such as natural killer (NK) cells, are potent anti-tumor effectors that play an important role in innate and adaptive immunity. There are three activating receptors found on NK cells, NKp30, NKp44, and NKp46, which are collectively known as Natural Cytotoxicity Receptors (NCRs). NKp46 is an established marker for the identification of NK cells. NKp46 is an NK cell specific triggering molecule found on both resting and activated NK cells. It is an important mediator in NK cell activation against numerous targets, including tumors and virally infected cells.

The use of immune cells for adoptive cell therapies remain to be challenging and have unmet needs for improvement. There are significant opportunities that remain to harness the full potential of NK cells, or other lymphocytes in adoptive immunotherapy. There is an unmet need to provide additional and more effective, specific, safe and/or stable agents that alone, as part of immunologic construct, or in combination with other agents, potentiate cells of the immune system, such as NK cells, to attack tumor cells. Accordingly, there exists a need for novel antibodies and therapeutics that enable dual targeting of NKp46 on NK cells and CD38 on tumor cells, for the treatment of cancers such as multiple myeloma and lymphoma.

### SUMMARY

The present disclosure provides antibodies capable of targeting NKp46 on NK cells and CD38 on tumor cells, for the treatment of cancers including but not limited to multiple myeloma (MM), lymphoma, and leukemias (e.g., acute myeloid leukemia).

The present disclosure provides a bispecific antibody that specifically binds NKp46 and CD38, comprising: i) a first heavy chain comprising a heavy chain complementarity determining region 1 (CDRH1) comprising an amino acid sequence of SEQ ID NO: 17; a heavy chain complementarity determining region 2 (CDRH2) comprising an amino acid sequence of SEQ ID NO: 18; and a heavy chain complementarity determining region 3 (CDRH3) comprising an amino acid sequence of SEQ ID NO: 19; ii) a first light chain comprising a light chain complementarity determining region 1 (CDRL1) comprising an amino acid sequence of SEQ ID NO: 20; a light chain complementarity determining region 2 (CDRL2) comprising an amino acid sequence of SEQ ID NO: 21; and a light chain complementarity determining region 3 (CDRL3) comprising an amino acid sequence of SEQ ID NO: 22; iii) a second heavy chain comprising a CDRH1 comprising an amino acid sequence of SEQ ID NO: 32; a CDRH2 comprising an amino acid sequence of SEQ ID NO: 33; and a CDRH3 comprising an amino acid sequence of SEQ ID NO: 34; and iv) a second light chain comprising a CDRL1 comprising an amino acid sequence of SEQ ID NO: 35; a CDRL2 comprising an amino acid sequence of SEQ ID NO: 36; and a CDRL3 comprising an amino acid sequence of SEQ ID NO: 37; and wherein the bispecific antibody comprises a first antigen binding region comprising i) and ii) that specifically binds to NKp46 and a second antigen binding region comprising iii) and iv) that specifically binds to CD38.

In some embodiments, the first heavy chain comprises a first heavy chain variable region comprising an amino acid sequence of SEQ ID NO: 23, 25, 27 or 29; the first light chain comprises a first light chain variable region comprising an amino acid sequence of SEQ ID NO: 24, 26, 28 or 30; the second heavy chain comprises a second heavy chain variable region comprising an amino acid sequence of SEQ ID NO: 38; and the second light chain comprises a second light chain variable region comprising an amino acid sequence of SEQ ID NO: 39.

In some embodiments, the first antigen binding region comprises a) a first heavy chain comprising a first heavy chain variable region comprising an amino acid sequence of SEQ ID NO: 23 and a first light chain comprising a first light chain variable region comprising the amino acid sequence of SEQ ID NO: 24; b) a first heavy chain comprising a first heavy chain variable region comprising an amino acid sequence of SEQ ID NO: 25 and a first light chain comprising a first light chain variable region comprising the amino acid sequence of SEQ ID NO: 26; c) a first heavy chain comprising a first heavy chain variable region comprising an amino acid sequence of SEQ ID NO: 27 and a first light chain comprising a first light chain variable region comprising the amino acid sequence of SEQ ID NO: 28; or d) a first heavy chain comprising a first heavy chain variable region comprising an amino acid sequence of SEQ ID NO: 29 and a first light chain comprising a first light chain variable region comprising the amino acid sequence of SEQ ID NO: 30.

In some embodiments, the second antigen binding region comprises a second heavy chain comprising a second heavy chain variable region comprising an amino acid sequence of SEQ ID NO: 38 and a second light chain comprising a second light chain variable region comprising the amino acid sequence of SEQ ID NO: 39.

In some embodiments, a) the first heavy chain comprises a first heavy chain variable region comprising an amino acid sequence of SEQ ID NO: 23; the first light chain comprises a first light chain variable region comprising an amino acid sequence of SEQ ID NO: 24; the second heavy chain comprises a second heavy chain variable region comprising an amino acid sequence of SEQ ID NO: 38; and the second light chain comprises a second light chain variable region comprising an amino acid sequence of SEQ ID NO: 39; b) the first heavy chain comprises a first heavy chain variable region comprising an amino acid sequence of SEQ ID NO: 25; the first light chain comprises a first light chain variable region comprising an amino acid sequence of SEQ ID NO: 26; the second heavy chain comprises a second heavy chain variable region comprising an amino acid sequence of SEQ ID NO: 38; and the second light chain comprises a second light chain variable region comprising an amino acid sequence of SEQ ID NO: 39; c) the first heavy chain comprises a first heavy chain variable region comprising an amino acid sequence of SEQ ID NO: 27; the first light chain comprises a first light chain variable region comprising an amino acid sequence of SEQ ID NO: 28; the second heavy chain comprises a second heavy chain variable region comprising an amino acid sequence of SEQ ID NO: 38; and the second light chain comprises a second light chain variable region comprising an amino acid sequence of SEQ ID NO: 39; or d) the first heavy chain comprises a first heavy chain variable region comprising an amino acid sequence of SEQ ID NO: 29; the first light chain comprises a first light chain variable region comprising an amino acid sequence of SEQ ID NO: 30; the second heavy chain comprises a second heavy chain variable region comprising an amino acid sequence of SEQ ID NO: 38; and the second light chain comprises a second light chain variable region comprising an amino acid sequence of SEQ ID NO: 39.

In some embodiments, the bispecific antibody comprises a fused heavy chain comprising an amino acid sequence of SEQ ID NO: 41, a first light chain comprising an amino acid sequence of SEQ ID NO: 31 and a second light chain comprising an amino acid sequence of SEQ ID NO: 40.

In some embodiments, the bispecific antibody comprises at least two Fab fragments with different CH1 and CL domains, wherein said Fab fragments comprise: a) a first Fab fragment consisting of i. the VH region and VL region that specifically binds NKp46; ii. a CH1 domain of a human immunoglobulin comprising substitution of the threonine residue at position 192 of said CH1 domain with a glutamic acid residue; and iii. a CL-kappa domain of a human immunoglobulin comprising substitution of the asparagine residue at position 137 of said CL domain with a lysine residue and substitution of the serine residue at position 114 of said CL domain with an alanine residue, b) a second Fab fragment consisting of wild-type human CH1 and wild-type human CL domains of an immunoglobulin, and the VH region and VL region that specifically binds CD38; and wherein the sequence position numbers used for the CH1 and CL domains refer to Kabat numbering and Fab fragments are tandemly arranged in any order, and wherein the C-terminal end of the CH1 domain of the first Fab fragment being linked to the N-terminal end of the VH domain of the following Fab fragment is through a polypeptide linker.

In some embodiments, the polypeptide linker comprises an amino acid sequence of SEQ ID NO: 9 or 44.

In some embodiments, the bispecific antibody further comprises c) a dimerized CH2 domain and CH3 domain of an immunoglobulin; and d) a hinge region of an IgA, IgG, or IgD, linking the C-terminal ends of the CH1 domains of the antigen binding region to the N-terminal ends of the CH2 domains.

In some embodiments, the bispecific antibody further comprises further comprising an Fc domain derived from an IgG1 Fc domain or an IgG4 Fc domain. In some embodiments, the Fc domain region comprises an amino acid sequence of SEQ ID NO: 15.

In some embodiments, the Fc domain region comprises an amino acid sequence of SEQ ID NO: 16.

In some embodiments, the bispecific antibody is a human antibody, a humanized antibody or a chimeric antibody. In some embodiments, the IgG antibody is an IgG1 or an IgG4 antibody.

The present disclosure also provides a nucleic acid sequence encoding any one of the bispecific antibodies of the disclosure.

The present disclosure also provides a multispecific antibody comprising the antigen binding regions of the bispecific antibodies of the disclosure.

In some embodiments, the multispecific antibody is fused or conjugated to a cytokine.

The present disclosure further provides a method of treating, preventing, or delaying the progression of pathologies associated with aberrant CD38 expression or activity in a subject in need thereof, comprising administering an effective amount of the bispecific antibodies or multispecific antibodies of the disclosure.

In some embodiments, the pathology is cancer. In some embodiments, the cancer is a multiple myeloma. In some embodiments, the cancer is a lymphoma. In some embodiments, the cancer is a leukemia (e.g., chronic myeloid leukemia).

The present disclosure further provides a method of redirecting a NK cell response in a subject in need thereof, comprising administering an effective amount of the bispecific antibodies or multispecific antibodies of the disclosure

In some embodiments, the NK cell response is NK-mediated cytotoxicity or antibody-dependent cellular cytotoxicity (ADCC).

The present disclosure further provides a method of promoting specific lysis of cells expressing CD38+ cells) by natural killer (NK) cells, comprising contacting the CD38+ cells with an effective amount of the bispecific antibodies or multispecific antibodies of the disclosure, wherein the effective amount is an amount sufficient to promote the specific lysis of the CD38+ cells by NK cells. In some embodiments, the CD38+ cells are multiple myeloma cells. In some embodiments, the CD38+ cells are lymphoma cells. In some embodiments, the contacting step comprises administering the bispecific antibody to a subject suffering from or a risk for multiple myeloma or lymphoma.

The present disclosure further provides a method of inhibiting proliferation of multiple myeloma or lymphoma cells or CD38+ cancer cells in a subject treated with a bispecific antibody according to any one of claims 1-15, comprising administering an effective amount of natural killer (NK) cell. In some embodiments, the method comprises administering an effective amount of natural killer (NK) cells.

The present disclosure further provides a combination therapy or a kit for treatment of multiple myeloma, lymphoma, a leukemia (e.g., acute myeloid leukemia) or a CD38+ cancer, comprising NK cells and a bispecific antibody of the disclosure.

The present disclosure further provides a bispecific antibody use in combination with NK cells.

The present disclosure also provides a use of natural killer (NK) cells and a bispecific antibody for treatment of multiple myeloma, lymphoma or a CD38+ cancer.

The present disclosure also provide a kit comprising a bispecific antibody of the disclosure.

In some embodiments, the NK cells are induced pluripotent stem cell-derived natural killer (iPSC-NK) cells. In some embodiments, the CD38+ cancer is multiple myeloma or lymphoma. In some embodiments, the NK cells are donor-derived NK cells. In some embodiments, the NK cells are irradiated immortalized NK cells.

### BRIEF DESCRIPTION OF THE DRAWINGS

FIG. 1A-1B shows a series of flow cytometry histograms depicting staining of cells expressing human NKp46 with mouse anti-NKp46 monoclonal antibodies. FIG. 1A shows FACs staining using anti-NKp46 mAbs (9E2, 461-G1, 02, 09, 12) of BW parental versus BW transfected cells expressing NKp46. The filled gray histogram represents staining with secondary antibody only of the BW parental cells. The background of BW NKp46 transfectants was similar and is not shown. These histograms correspond with one representative experiment out of 6 performed. FIG. 1B shows FACs staining using anti-NKp46 mAbs (9E2, 461-G1, 02, 09, 12) of primary activated bulk human NK cells. The filled gray histogram represents staining of NK cells with secondary antibody only. These histograms correspond with one representative experiment out of 6 performed.

FIG. 2 shows a series of flow cytometry histograms depicting the detection of NKp46 on three types of cells. NKp46-Ig was pre-incubated either alone or with anti-NKp46 mAbs (9E2, 461-G1, 02, 09, 12) at 4° C., followed by FACs staining of BJAB, MCF7 and C1R cells with the pre-treated NKp46-Ig. The filled gray histogram represents staining of cells with secondary antibody only. These histograms correspond with one representative experiment out of 2 performed.

FIG. 3 shows a series of flow cytometry histograms showing downregulation of surface expression of NKp46 following the binding of anti-NKp46 antibodies. Activated bulk NK cell cultures were incubated with the indicated anti-NKp46 mAbs (9E2, 461-G1, 02, 09, 12) at 4° C. The background of cells treated at 37° C. was similar and is not shown. These histograms correspond with one representative experiment out of 5 performed.

FIG. 4 shows a dose response FACS staining with these antibodies on two primary activated primary NK cells. FACs staining using anti-NKp46 mAbs (9E2, 461-G1, 02, 09, 12) of primary activated bulk human NK cells from two donors, NK1 and NK2. The filled gray histogram represents staining of NK cells with secondary antibody only.

FIG. 5 shows binding affinity of mouse anti-NKp46 mAb 09 determined by BIAcore assay.

FIG. 6 shows binding affinity of mouse anti-NKp46 mAb 12 determined by BIAcore assay.

FIG. 7A-7B shows amino acid sequence alignments of variable regions of mouse anti-NKp46 antibodies in comparison to humanized anti-NKp46 antibodies. FIG. 7A shows an amino acid sequence alignment of the heavy chain variable region. FIG. 7B shows an amino acid sequence alignment of kappa light chain variable region.

FIG. 8 shows full length human NKp46, NKp46 domain I (D1) and NKp46 domain II (D2).

FIG. 9 shows a series of histograms depicting the detection of NKp46 on BW cells, BW NKp46 cells and NK Fiji cells using anti-NKp46 mAb antibodies. A commercial anti-NKp46 antibody (black line) was tested (BioLegends (cat #331702)).

FIG. 10 shows a series of histograms depicting the detection of NKp46 on BW cells, BW NKp46 cells and NK Fiji cells using humanized NKp46 hybridoma antibodies.

FIG. 11 shows a graph depicting the activation and percentage of human NK cell killing of cells incubated with anti-NKp46 antibodies and humanized NKp46 hybridoma antibodies. PAR-R is a control antibody. GPC3 is anti GPC3 control antibody. 9E2 is a commercial anti-NKp46 antibody.

FIG. 12 shows a graph depicting the percentage of human NK cell killing of HepG2 cells following incubation with anti-NKp46 antibodies and humanized NKp46 hybridoma antibodies.

FIG. 13A-13B shows schematic diagrams of the bispecific antibody molecule or NK Engager Bispecific antibody of the disclosure. FIG. 13A shows a schematic diagram of the structure of the bispecific antibody. In some instances Mab1 is an anti-NKp46 Fab; Mab2 is an anti-CD38 Fab; Linker is a polypeptide linker; Hinge 1 and Hinge 2 are a human IgG1 or IgG4 hinge; Fc Region is a human IgG1 or a human IgG4 Fc region. Fab fragments have mutations (shown by circles) at the interface of the CH1 and CL domains that prevent heavy chain and light chain mispairing. FIG. 13B shows a schematic diagram of a bispecific antibody molecule or NK Engager bispecific antibody that binds NKp46 expressed on an NK cell and a tumor antigen (TA) such as CD38 expressed on the surface of a tumor cell. Binding of the bispecific antibody to both surface antigens causes NK-cell mediated cytotoxicity.

FIG. 14 shows dose-dependent binding of NKE2 to multiple myeloma cell lines.

FIG. 15A-15B show binding of NKE2 to BW parental cells and human Nkp46 transfected BW cells, respectively.

FIG. 16 shows results of a NKE2 epitope mapping analysis by CD38 alanine scanning mutagenesis.

FIG. 17A-17C show dose-dependent PBNK redirected cytolysis of multiple myeloma cell lines KMS 11 (FIG. 17A), MM.1S (FIG. 17B), and MM.1S (FIG. 17C) with NKE2.

FIGS. 18A and 18B show PB-NK cell redirected cytolysis, degranulation and cytokine production against multiple myeloma MM.1S cells. FIG. 18A shows PB-NK cell redirected cytolysis and degranulation against multiple myeloma MM.1S cells with NKE2, Daratumumab, or single CD38 and NKp46 mAb's. FIG. 18B shows proportion of IFN-γ and TNF-α producing PBNK cells with NKE2, Daratumumab and single CD38 and NKp46 mAbs.

FIG. 19A-19C show lack of immune subset depletion and low levels of NK cell fratricide and cytokine release with wildtype and mutant NKE2 in human PBMC's in-vitro.

FIG. 19A shows NK cell fratricide of PBNK's in the presence of NKE2, Daratumumab, or human IgG. FIG. 19B shows human PBMC immune cell subset depletion with NKE2, Daratumumab, or hIgG1. FIG. 19C shows cytokine release from human PBMCs after incubation with NKE2, anti-CD3 or CD28 mAbs (TGN1412), or hIgG1

### DETAILED DESCRIPTION

The present disclosure provides bispecific antibodies and antigen binding fragments thereof which bind to human natural killer receptor NKp46 and CD38. Specifically, the bispecific antibody includes a first antigen binding region that specifically binds NKp46 expressed on the surface of NK cells and a second antigen binding region that specifically binds to CD38 expressed on tumor cells. In the context of the present disclosure, the following definitions are provided.

### Definitions

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)). The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

As used in the specification and claims, the singular form “a”, “an” and “the” include plural references unless the context clearly dictates otherwise. For example, the term “a cell” includes a plurality of cells, including mixtures thereof.

As used herein, the “administration” of an agent, (e.g., an anti-NKp46 and anti-CD38 bispecific antibody), to a subject or subject includes any route of introducing or delivering to a subject a compound to perform its intended function. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated and target cell or tissue. Non-limiting examples of route of administration include parenteral, enteral, and topical routes of administration. Administration includes self-administration and the administration by another. It is also to be appreciated that the various modes of treatment or prevention of medical conditions as described are intended to mean “substantial”, which includes total but also less than total treatment or prevention, and wherein some biologically or medically relevant result is achieved.

As used herein, the term “animal” refers to living multi-cellular vertebrate organisms, a category that includes, for example, mammals and birds. The term “mammal” includes both human and non-human mammals. Similarly, the term “subject” or “patient” includes both human and veterinary subjects, for example, humans, non-human primates, dogs, cats, sheep, mice, horses, and cows.

As used herein, the term “antibody” refers to immunoglobulin molecules and immunologically active portions of immunoglobulin (Ig) molecules, i.e., molecules that contain an antigen binding site that specifically binds (immunoreacts with) an antigen. By “specifically bind” or “immunoreacts with” or “immunospecifically bind” is meant that the antibody reacts with one or more antigenic determinants of the desired antigen and does not react with other polypeptides or binds at much lower affinity (K_{D}>10⁻⁶ M). Antibodies include, but are not limited to, polyclonal, monoclonal, chimeric, dAb (domain antibody), single chain, Fab, Fab′ and F(ab′)2 fragments, scFvs, and an Fab expression library. Antibodies with high affinity, such as the antibodies described herein, have an affinity (K_{D} of about 0.01-25 nM or less.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. In general, antibody molecules obtained from humans relate to any of the classes IgG, IgM, IgA, IgE and IgD, which differ from one another by the nature of the heavy chain present in the molecule. Certain classes have subclasses as well, such as IgG1, IgG2, and others. Furthermore, in humans, the light chain may be a kappa chain or a lambda chain.

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs contain an antigen binding site capable of immunoreacting with a particular epitope of the antigen characterized by a unique binding affinity for it.

The term “antigen binding region” or “antigen-binding site” or “binding portion” refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions” or “FRs.” Thus, the term “FR” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions” or “CDRs.” Various methods are known in the art for numbering the amino acids sequences of antibodies and identification of the complementarity determining regions. For example, the Kabat numbering system (See Kabat, E. A., et al., Sequences of Protein of Immunological Interest, 5^{th} ed. (1991)) or the IMGT numbering system (IMGT®, the international ImMunoGeneTics information System©. Available online at www.imgt.org). The IMGT numbering system is routinely used and accepted as a reliable and accurate system in the art to determine amino acid positions in coding sequences, alignment of alleles, and to easily compare sequences in immunoglobulin (IG) and T-cell receptor (TR) from all vertebrate species. The accuracy and the consistency of the IMGT data are based on IMGT-ONTOLOGY, the first, and so far unique, ontology for immunogenetics and immunoinformatics (Lefranc. M. P. et al., Biomolecules, 2014 Dec.; 4(4), 1102-1139). IMGT tools and databases run against IMGT reference directories built from a large repository of sequences. In the IMGT system the IG V-DOMAIN and IG C-DOMAIN are delimited taking into account the exon delimitation, whenever appropriate. Therefore, the availability of more sequences to the IMGT database, the IMGT exon numbering system can be and “is used” by those skilled in the art reliably to determine amino acid positions in coding sequences and for alignment of alleles. Additionally, correspondences between the IMGT unique numbering with other numberings (i.e., Kabat) are available in the IMGT Scientific chart (Lefranc. M. P. et al., Biomolecules, 2014 Dec.; 4(4), 1102-1139).

The term “hypervariable region” or “variable region” refers to the amino acid residues of an antibody that are typically responsible for antigen-binding. The hypervariable region generally comprises amino acid residues from a “complementarity determining region” or “CDR” (e.g., around about residues 24-34 (LI), 50-56 (L2) and 89-97 (L3) in the VL, and around about 31-35 (HI), 50-65 (H2) and 95-102 (H3) in the VH when numbered in accordance with the Kabat numbering system; Kabat et al., Sequences of Proteins of Immunological Interest, 5th ed (1991)); and/or those residues from a “hypervariable loop” (e.g., residues 24-34 (LI), 50-56 (L2) and 89-97 (L3) in the VL, and 26-32 (HI), 52-56 (H2) and 95-101 (H3) in the VH when numbered in accordance with the Chothia numbering system; Chothia and Lesk, J. Mol. Biol. 196:901-917 (1987)); and/or those residues from a “hypervariable loop” VCDR (e.g., residues 27-38 (LI), 56-65 (L2) and 105-120 (L3) in the VL, and 27-38 (HI), 56-65 (H2) and 105-120 (H3) in the VH when numbered in accordance with the IMGT numbering system; Lefranc, M. P. et al. Nucl. Acids Res. 27:209-212 (1999), Ruiz, M. e al. Nucl. Acids Res. 28:219-221 (2000)). Optionally, the antibody has symmetrical insertions at one or more of the following points 28, 36 (LI), 63, 74-75 (L2) and 123 (L3) in the VL, and 28, 36 (HI), 63, 74-75 (H2) and 123 (H3) in the VH when numbered in accordance with AHo; Honneger, A. and Plunkthun, A. J. Mol. Biol. 309:657-670 (2001)).

“Antibody fragments” or “antigen binding fragments” include proteolytic antibody fragments (such as F(ab′)2 fragments, Fab′ fragments, Fab′-SH fragments and Fab fragments as are known in the art), recombinant antibody fragments (such as sFv fragments, dsFv fragments, bispecific sFv fragments, bispecific dsFv fragments, F(ab)′2 fragments, single chain Fv proteins (“scFv”), disulfide stabilized Fv proteins (“dsFv”), diabodies, and triabodies (as are known in the art), and camelid antibodies (see, for example, U.S. Pat. Nos. 6,015,695; 6,005,079; 5,874,541; 5,840,526; 5,800,988; and 5,759,808). An scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains.

As used herein, the term “antigen” refers to a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, and proteins. Common categories of antigens include, but are not limited to, viral antigens, bacterial antigens, fungal antigens, protozoa and other parasitic antigens, tumor antigens, antigens involved in autoimmune disease, allergy and graft rejection, toxins, and other miscellaneous antigens.

As used herein, the term “epitope” includes any protein determinant capable of specific binding to an immunoglobulin, an scFv, or a T-cell receptor. The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. For example, antibodies may be raised against N-terminal or C-terminal peptides of a polypeptide.

As used herein, the terms “immunological binding,” and “immunological binding properties” refer to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See Nature 361:186-87 (1993)). The ratio of K_{off}/Kₒₙ enables the cancellation of all parameters not related to affinity, and is equal to the dissociation constant K_{d}. (See, generally, Davies et al. (1990) Annual Rev Biochem 59:439-473). An antibody of the present invention is the to specifically bind to its target, when the equilibrium binding constant (K_{d}) is 1 μM, e.g., 100 nM, preferably 10 nM, and more preferably 1 nM, as measured by assays such as biolayer interferometry assays or similar assays known to those skilled in the art.

As used herein, “binding affinity” refers to the tendency of one molecule to bind (typically non-covalently) with another molecule, such as the tendency of a member of a specific binding pair for another member of a specific binding pair. A binding affinity can be measured as a dissociation constant, which for a specific binding pair (such as an antibody/antigen pair) can be lower than 1×10⁻⁵ M, lower than 1×10⁻⁶ M, lower than 1×10⁻⁷ M, lower than 1×10⁻⁸ M, lower than 1×10⁻⁹ M, lower than 1×10⁻¹⁰ M, lower than 1×10⁻¹¹ M or lower than 1×10⁻¹² M. In one aspect, binding affinity is calculated by a modification of the Scatchard method described by Frankel et al., Mol. Immunol., 16:101-106, 1979. In another aspect, binding affinity is measured by a binding constant. In another aspect, binding affinity is measured by an antigen/antibody dissociation rate. In yet another aspect, a high binding affinity is measured by a competition radioimmunoassay.

The term “isolated polynucleotide” as used herein shall mean a polynucleotide of genomic, cDNA, or synthetic origin or some combination thereof, which by virtue of its origin the “isolated polynucleotide” (1) is not associated with all or a portion of a polynucleotide in which the “isolated polynucleotide” is found in nature, (2) is operably linked to a polynucleotide which it is not linked to in nature, or (3) does not occur in nature as part of a larger sequence. Polynucleotides in accordance with the invention include the nucleic acid molecules encoding the heavy chain immunoglobulin molecules, and nucleic acid molecules encoding the light chain immunoglobulin molecules described herein.

The term “isolated protein” referred to herein means a protein of cDNA, recombinant RNA, or synthetic origin or some combination thereof, which by virtue of its origin, or source of derivation, the “isolated protein” (1) is not associated with proteins found in nature, (2) is free of other proteins from the same source, e.g., free of marine proteins, (3) is expressed by a cell from a different species, or (4) does not occur in nature.

The term “polypeptide” is used herein as a generic term to refer to native protein, fragments, or analogs of a polypeptide sequence. Hence, native protein fragments, and analogs are species of the polypeptide genus. Polypeptides in accordance with the invention comprise the heavy chain immunoglobulin molecules, and the light chain immunoglobulin molecules described herein, as well as antibody molecules formed by combinations comprising the heavy chain immunoglobulin molecules with light chain immunoglobulin molecules, such as kappa light chain immunoglobulin molecules, and vice versa, as well as fragments and analogs thereof.

The phrase “naturally occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally-occurring.

The term “operably linked” as used herein refers to positions of components so described are in a relationship permitting them to function in their intended manner. A control sequence “operably linked” to a coding sequence is ligated in such a way that expression of the coding sequence is achieved under conditions compatible with the control sequences.

The term “control sequence” as used herein refers to polynucleotide sequences which are necessary to effect the expression and processing of coding sequences to which they are ligated. The nature of such control sequences differs depending upon the host organism in prokaryotes, such control sequences generally include promoter, ribosomal binding site, and transcription termination sequence in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences. The term “polynucleotide” as referred to herein means a polymeric boron of nucleotides of at least 10 bases in length, either ribonucleotides or deoxynucleotides or a modified form of either type of nucleotide. The term includes single and double stranded forms of DNA.

As used herein, the twenty conventional amino acids and their abbreviations follow conventional usage. See Immunology—A Synthesis (2nd Edition, E. S. Golub and D. R. Gren, Eds., Sinauer Associates, Sunderland Mass. (1991)). Stereoisomers (e.g., D-amino acids) of the twenty conventional amino acids, unnatural amino acids such as α-, α-disubstituted amino acids, N-alkyl amino acids, lactic acid, and other unconventional amino acids may also be suitable components for polypeptides of the present invention. Examples of unconventional amino acids include: 4 hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity, and most preferably at least 99 percent sequence identity.

Preferably, residue positions which are not identical differ by conservative amino acid substitutions.

Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine valine, glutamic-aspartic, and asparagine-glutamine.

As discussed herein, minor variations in the amino acid sequences of antibodies or immunoglobulin molecules are contemplated as being encompassed by the present invention, providing that the variations in the amino acid sequence maintain at least 75%, more preferably at least 80%, 90%, 95%, and most preferably 99%. In particular, conservative amino acid replacements are contemplated. Conservative replacements are those that take place within a family of amino acids that are related in their side chains. Genetically encoded amino acids are generally divided into families: (1) acidic amino acids are aspartate, glutamate; (2) basic amino acids are lysine, arginine, histidine; (3) non-polar amino acids are alanine, valine, leucine, isoleucine, proline, phenylalanine, methionine, tryptophan, and (4) uncharged polar amino acids are glycine, asparagine, glutamine, cysteine, serine, threonine, tyrosine. The hydrophilic amino acids include arginine, asparagine, aspartate, glutamine, glutamate, histidine, lysine, serine, and threonine. The hydrophobic amino acids include alanine, cysteine, isoleucine, leucine, methionine, phenylalanine, proline, tryptophan, tyrosine and valine. Other families of amino acids include (i) serine and threonine, which are the aliphatic-hydroxy family; (ii) asparagine and glutamine, which are the amide containing family; (iii) alanine, valine, leucine and isoleucine, which are the aliphatic family; and (iv) phenylalanine, tryptophan, and tyrosine, which are the aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, the foregoing examples demonstrate that those of skill in the art can recognize sequence motifs and structural conformations that may be used to define structural and functional domains in accordance with the invention.

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various muteins of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H.

Freeman and Company, New York (1984)); Introduction to Protein Structure (C. Branden and J. Tooze, eds., Garland Publishing, New York, N.Y. (1991)); and Thornton et at. Nature 354:105 (1991).

As used herein, the terms “label” or “labeled” refers to incorporation of a detectable marker, e.g., by incorporation of a radiolabeled amino acid or attachment to a polypeptide of biotinyl moieties that can be detected by marked avidin (e.g., streptavidin containing a fluorescent marker or enzymatic activity that can be detected by optical or calorimetric methods). In certain situations, the label or marker can also be therapeutic. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, p-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance. The term “pharmaceutical agent or drug” as used herein refers to a chemical compound or composition capable of inducing a desired therapeutic effect when properly administered to a patient.

Other chemistry terms herein are used according to conventional usage in the art, as exemplified by The McGraw-Hill Dictionary of Chemical Terms (Parker, S., Ed., McGraw-Hill, San Francisco (1985)).

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present.

Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

The terms “cancer,” “neoplasm,” and “tumor,” used interchangeably and in either the singular or plural form, refer to cells that have undergone a malignant transformation that makes them pathological to the host organism. Non-limiting examples of cancers that may be treated according to the methods of the present disclosure include hematological malignancies and solid tumors. Non-limiting examples of cancers include multiple myeloma. lymphoma, and leukemias (e.g., AML).

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of this disclosure, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. Preferred are compounds that are potent and can be administered locally at very low doses, thus minimizing systemic adverse effects.

### Anti-NKp46 and Anti-CD38 Bispecific Antibodies

The bispecific antibodies of the present disclosure are advantageous over those in the art because they target NKp46 to engage NK cells, rather than other NK cells markers such as CD16 and NKG2D. Importantly, NKp46 expression is frequently maintained in solid tumors with downregulated CD16 and NKG2D. As such, the bispecific antibodies of the present disclosure may be more effective than other bispecific antibody therapies known in the art targeting other lower expression markers. Furthermore, NKp46 is more specific to NK cells. In contrast, NKG2D is widely expressed by T cells, leading to toxicities such as cytokine release syndrome (CRS) when it is targeted using bispecifics. As such, bispecific antibodies of the present disclosure have a better safety profile than other bispecific antibodies known in the art that target other NK cell markers.

The bispecific antibodies of the present disclosure bind specifically to the NKp46 membrane proximal domain (D2 domain) which is advantageous because it does not block NKp46 interactions with its ligands. The bispecific antibodies do not internalize or degrade the NKp46 receptor and therefore can be used for recruiting NK cells in a variety of therapies.

Accordingly, the bispecific antibodies are useful in cancer immunotherapy and may also be used for cancer diagnosis.

### Human Natural Killer Receptor NKp46

The term “NKp46” as used herein refers to a natural killer protein 46, also known as Natural cytotoxicity triggering receptor 1 (NCR1) or CD335. NKp46 is a NK cell specific triggering molecule found on both resting and activated NK cells (Sivori et al., 1997). It is an important mediator in NK cell activation against numerous targets, including tumors and virally infected cells (Moretta et al., 2001). NKp46 is the only receptor on NK cells to have a mouse ortholog, denoted NCR1 (Biassoni et al., 1999). NKp46 is an established marker for the identification of NK cells (Koch et al., 2013).

NKp46 has two Ig-like extracellular domains (D1 and D2) followed by a ˜40-residue stalk region, a type I transmembrane domain, and a short cytoplasmic tail. NKp46 is a major NK cell activating receptor that is involved in the elimination of HCV and other viral infected cells and has been shown to regulate interactions of NK cells with other immune cells including T cells and dendritic cells (DC). An exemplary NKp46 according to the invention is set forth in UniProt and GenBank symbols or accession numbers: UniProtKB-O76036 (NCTR1 HUMAN) and Gene ID: 9437. NKp46 has two Ig-like extracellular domains (D1 and D2) followed by a ˜40-residue stalk region, a type I transmembrane domain, and a short cytoplasmic tail. D2 domain (or NKp46D2), comprising 134 amino acid residues (corresponding to residues 121-254 of the full-length protein of isoform a).

The bispecific antibodies or antigen binding fragments thereof according to the invention bind to an epitope in NKp46. Specifically, the antibodies bind to an epitope within the D2 domain of the NKp46 protein.

### CD38

As used herein, the terms “CD38” and “CD38 antigen” are used interchangeably, and include any variants, isoforms and species homologs of human CD38, which are naturally expressed by cells or are expressed on cells transfected with the CD38 gene. Synonyms of CD38, as recognized in the art, include ADP ribosyl cyclase 1, cADPr hydrolase 1, Cd38-rsl, Cyclic ADP-ribose hydrolase 1, I-19, NIM-R5 antigen. The complete amino acid sequence of an exemplary human CD38 Genbank/NCBI accession number NP_001766.2 (UniProtID number P28907).

CD38 is a multifunctional protein having function in receptor-mediated adhesion and signaling as well as mediating calcium mobilization via its ecto-enzymatic activity, catalyzing formation of cyclic ADP-ribose (cADPR) and ADPR. CD38 mediates cytokine secretion and activation and proliferation of lymphocytes (Funaro et al., J Immunolog 145:2390-6, 1990; Terhorst et al., Cell 771-80, 1981; Guse et al., Nature 398:70-3, 1999). CD38, via its NAD glycohydrolase activity, also regulates extracellular NAD⁺ levels, which have been implicated in modulating the regulatory T-cell compartment (Adriouch et al., 14:1284-92, 2012; Chiarugi et al., Nature Reviews 12:741-52, 2012). In addition to signaling via Ca²⁺, CD38 signaling occurs via cross-talk with antigen-receptor complexes on T- and B-cells or other types of receptor complexes, e.g., MHC molecules, involving CD38 in several cellular responses, but also in switching and secretion of IgG1.

CD38 is a type II transmembrane glycoprotein expressed on hemopoietic cells such as medullary thymocytes, activated T- and B-cells, resting NK cells and monocytes, lymph node germinal center lymphoblasts, plasma B cells, intrafollicular cells and dendritic cells. A portion of normal bone marrow cells, particular precursor cells as well as umbilical cord cells are CD38-positive. In addition to lymphoid precursor cells, CD38 is expressed on erythrocytes and on platelets, and expression is also found in some solid tissues such as gut, brain, prostate, bone, and pancreas. Mature resting T- and B-cells express limited to no surface CD38.

CD38 is also expressed in a variety of malignant hematological diseases, including multiple myeloma, leukemias and lymphomas, such as B-cell chronic lymphocytic leukemia, T- and B-cell acute lymphocytic leukemia, Waldenstrom macroglobulinemia, primary systemic amyloidosis, mantle-cell lymphoma, pro-lymphocytic/myelocytic leukemia, acute myeloid leukemia, chronic myeloid leukemia, follicular lymphoma, Burkitt's lymphoma, large granular lymphocytic (LGL) leukemia, AML, NK-cell leukemia and plasma-cell leukemia. Expression of CD38 has been described on epithelial/endothelial cells of different origin, including glandular epithelium in prostate, islet cells in pancreas, ductal epithelium in glands, including parotid gland, bronchial epithelial cells, cells in testis and ovary and tumor epithelium in colorectal adenocarcinoma. Other diseases, where CD38 expression may be involved, include, e.g., broncho-epithelial carcinomas of the lung, breast cancer (evolving from malignant proliferation of epithelial lining in ducts and lobules of the breast), pancreatic tumors evolving from the β-cells (insulinomas), tumors evolving from epithelium in the gut (e.g., adenocarcinoma and squamous cell carcinoma), carcinoma in the prostate gland, and seminomas in testis and ovarian cancers. In the central nervous system, neuroblastomas express CD38.

### Bispecific Antibodies

The bispecific antibodies of the disclosure have one antigen binding region that is specific for NKp46 and a second antigen binding region that is specific for CD38.

While antibody sequences below are provided herein as examples, it is to be understood that these sequences can be used to generate bispecific antibodies using any of a variety of art-recognized techniques. Examples of bispecific formats include but are not limited to bispecific IgG based on Fab arm exchange (Gramer et al., 2013 MAbs. 5(6)); the CrossMab format (Klein C et al., 2012 MAbs 4(6)); multiple formats based on forced heterodimerization approaches such as SEED technology (Davis J H et al., 2010 Protein Eng Des Sel. 23(4):195-202), electrostatic steering (Gunasekaran K et al., J Biol Chem. 2010 285(25):19637-46.) or knob-into-hole (Ridgway J B et al., Protein Eng. 1996 9(7):617-21.) or other sets of mutations preventing homodimer formation (Von Kreudenstein T S et al., 2013 MAbs. 5(5):646-54.); fragment based bispecific formats such as tandem scFv (such as BiTEs) (Wolf E et al., 2005 Drug Discov. Today 10(18):1237-44.); bispecific tetravalent antibodies (Portner L M et al., 2012 Cancer Immunol Immunother. 61(10):1869-75.); dual affinity retargeting molecules (Moore P A et al., 2011 Blood. 117(17):4542-51), diabodies (Kontermann R E et al., Nat Biotechnol. 1997 15(7):629-31).

Bispecific or multi-specific engagers are fusion proteins consisting of two or more single-chain variable fragments (scFvs) of different antibodies, with at least one scFv binds to an effector cell surface molecule, and at least another to a tumor cell via a tumor specific surface molecule.

The exemplary NK cell surface molecules, that can be used for bi- or multispecific engager recognition, or coupling, include, but are not limited to, CD3, CD28, CD5, CD16, NKG2D, CD64, CD32, CD89, NKG2C.

The exemplary tumor cell surface molecules for bi-specific or multi-specific engager recognition include, but are not limited to, GPC3, B7H3, BCMA, CD10, CD19, CD20, CD22, CD24, CD30, CD33, CD34, CD38, CD44, CD79a, CD79b, CD123, CD138, CD179b, CEA, CLEC12A, CS-1, DLL3, EGFR, EGFRvIII, EPCAM, FLT-3, FOLR1, FOLR3, GD2, gpA33, HER2, HM1.24, LGR5, MSLN, MCSP, MICA/B, PSMA, PAMA, P-cadherin, ROR1.

In some embodiments, the bispecific antibody further comprises a linker between the effector cell and tumor cell antigen binding domains, for example, a modified IL15 as a linker for effector NK cells to facilitate effector cell expansion (called TriKE, or Trispecific Killer Engager, in some publications). In one embodiment, the TriKE is NKp46-IL15-CD38.

In some embodiments, the surface triggering receptor for bi- or multi-specific engager could be endogenous to the effector cells, sometimes depending on the cell types. In some other embodiments, one or more exogenous surface triggering receptors could be introduced to the effector cells using the methods and compositions provided herein, i.e., through additional engineering of an iPSC, then directing the differentiation of the iPSC to T, NK or any other effector cells comprising the same genotype and the surface triggering receptor as the source iPSC.

In some embodiments, the bispecific format includes but are not limited to the bispecific antibody format described in PCT application number WO2013005194, and US patent numbers U.S. Pat. Nos. 9,631,031 and 10,815,310, each of which are incorporated herein in their entirety. Sequence position numbers used herein for the CH1 and CL domains refer to Kabat numbering (Kabat, E. A. et al., Sequences of proteins of immunological interest. 5th Edition-US Department of Health and Human Services, NIH publication no 91-3242, pp 662,680,689, 1991) An bispecific antibody of the present invention is a mutated Fab fragment selected among: a) a Fab fragment consisting of: the VH and VL domains of an antibody of interest; a CH1 domain which is derived from the CH1 domain of an immunoglobulin by substitution of the threonine residue at position 192 of said CH1 domain with a glutamic acid residue; and a CL domain which is derived from the CL domain of an immunoglobulin by substitution of the asparagine residue at position 137 of said CL domain with a lysine residue and substitution of the serine residue at position 114 of said CL domain with an alanine residue; b) a Fab fragment consisting of: the VH and VL domains of an antibody of interest; a CH1 domain which is derived from the CH1 domain of an immunoglobulin by substitution of the leucine residue at position 143 of said CH1 domain with a glutamine residue and substitution of the serine residue at position 188 of said CH1 domain with a valine residue; and a CL domain which is derived from the CL domain of an immunoglobulin by substitution of the valine residue at position 133 of said CL domain with a threonine residue and substitution of the serine residue at position 176 of said CL domain with an valine residue. c) a Fab fragment consisting of: the VH and VL domains of an antibody of interest; a CH1 domain which is derived from the CH1 domain of an IgG immunoglobulin by substitution of the leucine residue at position 124 of said CH1 domain with an alanine residue and substitution of the leucine residue at position 143 of said CH1 domain with a glutamic acid residue; a CL domain which is derived from the CL domain of an IgG immunoglobulin by substitution of the valine residue at position 133 of said CL domain with a tryptophane residue; d) a Fab fragment consisting of: the VH and VL domains of an antibody of interest; a CH1 domain which is derived from the CH1 domain of an immunoglobulin by substitution of the valine residue at position 190 of said CH1 domain with an alanine residue; and a CL domain which is derived from the CL domain of an immunoglobulin by substitution of the leucine residue at position 135 of said CL domain with a tryptophan residue, and substitution of the asparagine residue at position 137 of said CL domain with an alanine residue.

According to a preferred embodiment, the CH1 domain is derived from a IgG immunoglobulin. In some embodiments, the IgG immunoglobulin is from IgG1 isotype or the IgG4 isotype. In some embodiments, the CL domain is a kappa type. For use in human therapy, the immunoglobulin from which the mutated CH1 and CL domains are derived is a human immunoglobulin.

Fab fragments being tandemly arranged in any order, the C-terminal end of the CH1 domain of a first Fab fragment being linked to the N-terminal end of the VH domain of the following Fab fragment through a polypeptide linker. Generally, said polypeptide linker should have a length of at least 20, preferably at least 25, and still more preferably at least 30, and up to 80, preferably up to 60, and still more preferably up to 40 amino-acids.

The polypeptide linker comprises all or part of the sequence of the hinge region of one or more immunoglobulin(s) selected among IgA, IgG, and IgD. If the antibody is to be used in human therapy, hinge sequences of human origin will be preferred.

Sequences of the hinge regions of human IgG, IgA and IgD are indicated below:

IgA1
(SEQ ID NO: 1):
VPSTPPTPSPSTPPTPSPS
IgA2
(SEQ ID NO: 2):
VPPPPP
IgD
(SEQ ID NO: 3):
ESPKAQASSVPTAQPQAEGSLAKATTAPATTRNTGRGGEEKKKEKEKEEQ
EERETKTP
IgG1
(SEQ ID NO: 4):
EPKSCDKTKTCPPCP
IgG2
(SEQ ID NO: 5):
ERKCCVECPPCP
IgG3:
(SEQ ID NO: 6)
ELKTPLGDTTHTCPRCP
followed by 0 or 1 to 4 repeats of
(SEQ ID NO: 7)
EPKSCDTPPPCPRCP.
IgG4:
(SEQ ID NO: 8)
ESKYGPPCPSCP

Said polypeptide linker may comprise all or part of the sequence of the hinge region of only one immunoglobulin. In this case, said immunoglobulin may belong to the same isotype and subclass as the immunoglobulin from which the adjacent CH1 domain is derived, or to a different isotype or subclass.

Alternatively, said polypeptide linker may comprise all or part of the sequences of hinge regions of at least two immunoglobulins of different isotypes or subclasses. In this case, the N-terminal portion of the polypeptide linker, which directly follows the CH1 domain, preferably consists of all or part of the hinge region of an immunoglobulin belonging to the same isotype and subclass as the immunoglobulin from which said CH1 domain is derived.

Optionally, said polypeptide linker may further comprise a sequence of from 2 to 15, preferably of from 5 to 10 N-terminal amino-acids of the CH2 domain of an immunoglobulin.

In some cases, sequences from native hinge regions can be used; in other cases point mutations can be brought to these sequences, in particular the replacement of one or more cysteine residues in native IgG1, IgG2 or IgG3 hinge sequences by alanine or serine, in order to avoid unwanted intra-chain or inter-chains disulfide bonds.

Non-limitative examples of polypeptide linkers which can be used in a multispecific antigen-binding fragment of the invention are polypeptides having the sequence EPKSCDKTHTCPPCPAPELLGGPSTPPTPSPSGG (SEQ ID NO: 9) or EPKSCDKTHTCPPCPAPELLGGPGGGGSGGSGSGG (SEQ ID NO: 44) or a sequence at least 80%, at least 90%, at least 95%, at least 96%, at least 97%, at least 98%, or at least 99% identical thereto. SEQ ID NO: 9 consists of the full length sequence of human IgG1 hinge (SEQ ID NO: 4), followed by the 9 N-terminal amino-acids of human IgG1 CH2 (APELLGGPS, SEQ ID NO: 10), by a portion of the sequence of human IgA1 hinge (TPPTPSPS, SEQ ID NO: 11), and by the dipeptide GG, added to provide supplemental flexibility to the linker. Other flexible linkers may be used, including GS type linkers, such as (GGGS)n or (GGGGS)n, wherein n is a integer, or non-repetitive linker, such as SPNSASHSGSAPQTSSAPGSQ (SEQ ID NO: 45) or a sequence at least 80%, at least 90%, at least 95%, at least 96%, at least 97%, at least 98%, or at least 99% identical thereto.

Optionally, a shorter portion of the N-terminal sequence of the human IgG1 CH2 domain can be used. Also, a longer portion of human IgA1 hinge, up to its full-length sequence (preferably minus the N-terminal valine residue) can be used. According to a particular embodiment, said human IgA1 hinge sequence can be replaced by an artificial sequence, containing an alternation of threonine, serine and proline residues.

For instance, a variant of the polypeptide of SEQ ID NO: 9, which is also suitable for use in a multispecific antigens-binding fragment of the invention is a polypeptide having the following sequence: EPKSCDKTHTCPPCPAPELLPSTPPSPSTPGG (SEQ ID NO: 12) or a sequence at least 80%, at least 90%, at least 95%, at least 96%, at least 97%, at least 98%, or at least 99% identical thereto. In this polypeptide, the full length sequence of human IgG1 hinge is followed by the 5 N-terminal amino-acids of human IgG1 CH2 (APELL, SEQ ID NO: 13), and by the sequence PSTPPSPSTP (SEQ ID NO: 14).

In case of a multispecific antigens-binding fragment of the invention, comprising more than two different Fab fragments, the polypeptide linkers separating the Fab fragments can be identical or different.

In some embodiments, the Fc domain is derived from a IgG immunoglobulin. In some embodiments, the IgG immunoglobulin is from IgG1 isotype or the IgG4 isotype. In some embodiments, the IgG1 Fc domain comprises a L234A and/or L235A mutation (EU numbering). For use in human therapy, the immunoglobulin from which the mutated Fc domains are derived is a human immunoglobulin.

In some embodiments, the IgG4 Fc domain comprises a S228P mutation (EU numbering). In some embodiments, the IgG4 Fc domain comprises an amino acid sequence of SEQ ID NO: 15 or a sequence at least 80%, at least 90%, at least 95%, at least 96%, at least 97%, at least 98%, or at least 99% identical thereto.

>mutIGHG4 S228P mutant of IgG4 isotype
(SEQ ID NO: 15)
ASTKGPSVFPLAPCSRSTSESTAALGCLVKDYFPEPVTVSWNSGALTSGVHTFPAVL

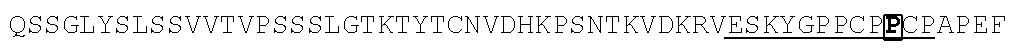

LGGPSVFLFPPKPKDTLMISRTPEVTCVVVDVSQEDPEVQFNWYVDGVEVHNAKT
KPREEQFNSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGK

In some embodiments, the IgG1 Fe domain comprises a L234A/L235A mutation (EU numbering). In some embodiments, the IgG1 Fe domain comprises an amino acid sequence of SEQ ID NO: 16 or a sequence at least 80%, at least 90%, at least 95%, at least 96%, at least 97%, at least 98%, or at least 99% identical thereto.

>IGHG1*01-L234A/L235A|Homo sapiens
(SEQ ID NO: 16)
ASTKGPSVFPLAPSSKSTSGGTAALGCLVKDYFPEPVTVSWNSGALTSGVHTFPAVL

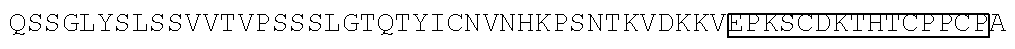

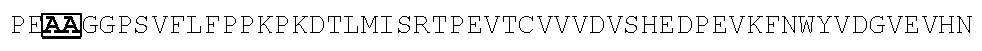

AKTKPREEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK*

>In some embodiments, the igG1 Fc domain is a wildtype igG1 Fc domain. In some embodiments, the IgG1 Fc domain comprises the sequence of SEQ ID NO: 42 or a sequence at least 80%, at least 90%, at least 95%, at least 96%, at least 97%, at least 98%, or at least 99% identical thereto.

>IGHG1*01 wildtype (human):
(SEQ ID NO: 42)
GPSVFPLAPSSKSTSGGTAALGCLVKDYFPEPVTVSWNSGALTSGVHTFPAVLQSSG

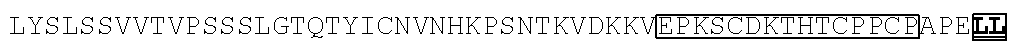

GGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDG
SFFLYSKLTVDKSRWQQGNVFSCSVMHEALHNHYTQKSLSLSPGK

An exemplary bispecific antibody of the disclosure comprises a CR3 mutation to control heavy and light chain pairing. (Golay et al. J Immunol. 2016). The bispecific antibody has a CH1 domain is of IgG1 isotype and comprises a CR3 mutation of T192E, light chain of the kappa isotype and comprising CR3 mutations of S114A and N137K, a peptidic linker of the GS type, a hinge domain, and an IgG4 Fc domain with a S228P (EU numbering) mutation.

An exemplary bispecific antibody of the disclosure comprises a CR3 mutation to control heavy and light chain pairing. (Golay et al. J Immunol. 2016). The bispecific antibody has a CH1 domain is of IgG1 isotype and comprises a CR3 mutation of T192E, light chain of the kappa isotype and comprising CR3 mutations of S114A and N137K, a peptidic linker of the GS type, a hinge domain, and an huIgG1 Fc domain with a L234A/L235A (EU numbering) mutation (Xu et al. In Vitro Characterization of Five Humanized OKT3 Effector Function Variant Antibodies. Cellular Immunology 200, 16-26 (2000). Hezareh et al. Effector Function Activities of a Panel of Mutants of a Broadly Neutralizing Antibody against Human Immunodeficiency Virus Type 1. J. Virol. 2001, 12161-12168 (75).

Exemplary Bispecific Antibodies that Specifically Bind to NKp46 and CD38

The bispecific antibodies of the disclosure have one antigen binding region that is specific for NKp46 and a second antigen binding region that is specific for CD38.

Exemplary NKp46 antibodies from which the NKp46 antigen binding region can be derived from include the 02 antibody, the 09 antibody (also referred to as “K3_P4” or “P4”), the 12 antibody (also referred to as “K3b” or “K3”), the humanized 09 antibody, the humanized 12 antibody, the B341001 antibody, the B341002 antibody, the B341003 antibody, and the B341004 antibody.

Exemplary, CD38 antibodies from which the CD38 antigen binding region can be derived from include the “anti-CD38” antibody. Additionally, exemplary CD38 antibodies from which the CD38 antigen binding region can be derived include but are not limited to those disclosed in U.S. Pat. Nos. 9,758,590, 9,944,711 and 10,766,965.

In some embodiments, exemplary bispecific antibodies of the invention that include at least a first antigen binding region that binds NKp46 and a second antigen binding region that binds CD38 include a combination of heavy chain and complementarity determining regions and light chain complementarity determining regions (CDRs) selected from the CDR sequences shown in Tables 1, 2, 3 and 4. The CDRs shown in Tables 1, 2, 3 and 4 are defined according to the IMGT nomenclature (See IMGT*, the international ImMunoGeneTics information System®. Available online: http://www.imgt.org/).

In some embodiments, exemplary bispecific antibodies of the invention that includes a first heavy chain comprising a combination of heavy chain CDR amino acid sequences selected from the CDRH1, CDRH2 and CDRH3 amino acid sequences shown in Table 1 and at a first light chain with a set of first light chain CDR amino acid sequences selected from the CDRL1, CDRL2 and CDRL3 amino acid sequences shown in Tables 2, a second heavy chain comprising a combination of heavy chain CDR amino acid sequence selected from CDRH1, CDRH2 and CDRH3 amino acid sequences shows in Table 3 and a second light chain with a set of second light chain CDR amino acid sequences selected form from CDRL1, CDRL2 and CDRL3 sequences Table 4.

In some embodiments, exemplary bispecific antibodies of the invention that include a first antigen binding region that binds NKp46 and a second antigen binding region that binds CD38, wherein the first antigen binding region includes the combination of heavy chain complementarity determining regions (CDRs) shown in Table 1 and a combination of the light chain CDRs selected from the CDR sequences shown in Table 2, and wherein the second antigen binding region includes the combination of heavy chain complementarity determining regions (CDRs) shown in Table 3 and a combination of the light chain CDRs selected from the CDR sequences shown in Table 4.

TABLE 1
Anti-NKp46 Heavy Chain CDRs (IMGT numbering)

| Ab ID | CDRH1 | CDRH2 | CDRH3 |
|---|---|---|---|
| >Heavy_09 | GYTFTEYS | ISPNSGGT | ARRDFHSSFDY |
| antibody | (SEQ ID | (SEQ ID | (SEQ ID NO: |
| | NO: 17) | NO: 18) | 19) |
| >Heavy_09 | GYTFTEYS | ISPNSGGT | ARRDFHSSFDY |
| antibody_ | (SEQ ID | (SEQ ID | (SEQ ID NO: |
| humanized | NO: 17) | NO: 18) | 19) |

TABLE 2
Anti-NKp46 Light Chain CDRs (IMGT Numbering)

| Ab ID | CDRL1 | CDRL2 | CDRL3 |
|---|---|---|---|
| >Light_09 | QSISDY | YAS | QNGHSFPLT |
| antibody | (SEQ ID | (SEQ ID | (SEQ ID |
| | NO: 20) | NO: 21) | NO: 22) |
| >Light_09 | QSISDY | YAS | QNGHSFPLT |
| antibody_ | (SEQ ID | (SEQ ID | (SEQ ID |
| humanized | NO: 20) | NO: 21) | NO: 22) |
| >Light_12 | QSISDY | YAS | QNGHSFPLT |
| antibody | (SEQ ID | (SEQ ID | (SEQ ID |
| | NO: 20) | NO: 21) | NO: 22) |
| >Light_12 | QSISDY | YAS | QNGHSFPLT |
| antibody_ | (SEQ ID | (SEQ ID | (SEQ ID |
| Humanized | NO: 20) | NO: 21) | NO: 22) |

TABLE 3
Anti-CD38 Heavy Chain CDRs (IMGT Numbering)

| Ab ID | CDRH1 | CDRH2 | CDRH3 |
|---|---|---|---|
| >Anti- | GYTFTSYW | IYPGDGDT | ARERTTGAPRYFDV |
| CD38 VH1 | (SEQ ID | (SEQ ID | (SEQ ID NO: 34) |
| | NO: 32) | NO: 33) | |

TABLE 4
Anti-CD38 Light Chain CDRs (IMGT Numbering)

| Name | CDRL1 | CDRL2 | CDRL3 |
|---|---|---|---|
| >Anti- | ENIYSF | NTK | QHHYGIPLT |
| CD38_VH1 | (SEQ ID | (SEQ ID | (SEQ ID |
| | NO: 35) | NO: 36) | NO: 37) |

Each of the exemplary anti-NKp46 and anti-CD38 bispecific antibodies described below includes a first heavy chain variable domain (VH), a first light chain variable domain (VL), a second heavy chain variable domain and a second light chain variable domain, as shown in the amino acid and corresponding nucleic acid sequences listed below.

### Anti-NKT46 Antibodies

Exemplary anti-NKp46 antibody sequences are shown below. Table 5 provides illustrative heavy chain variable and light chain variable amino acid sequences for anti-NKp46 antibodies according to the disclosure. Table 6 provides illustrative heavy chain variable and light chain variable nucleic acid sequences that encode the anti-NKp46 antibodies according to the disclosure.

TABLE 5
Exemplary VH and VL region amino acid
sequences of anti-NKp46 antibodies

| | | SEQ ID | | SEQ ID |
|---|---|---|---|---|
| Ab ID | VH full length | NO | VL full length | NO |
| >09 | EVQLQQSGPELVKPGTSV | 23 | DIVMTQSPATLSVTPGDR | 24 |
| antibody | KISCKTSGYTFTEYSMHW | | VSLSCRASQSISDYLHWF | |
| | VKQSHGKSLEWFGGISPN | | QQKSHESPRLLIKYASQS | |
| | SGGTSYNQKFKGKATLTV | | ISGIPSRFSGSGSGSDFT | |
| | DKSSSTAYMELRSLTSED | | LSINSVEPEDVGVYYCQN | |
| | SAVYYCARRDFHSSFDYW | | GHSFPLTFGPGTKLELK | |
| | GQGTTLTVSS | | | |
| >09 | EVQLQQSGAEVKKPGSSV | 25 | DIVMTQSPATLSLSPGER | 26 |
| antibody_ | KVSCKTSGYTFTEYSMHW | | ATLSCRASQSISDYLHWF | |
| humanized | VRQAPGKSLEWFGGISPN | | QQKPHESPRLLIKYASQS | |
| | SGGTSYNQKFKGRATLTV | | ISGIPARFSGSGSGSDFT | |
| | DKSSSTAYMELSSLRSED | | LTISSLEPEDFAVYYCQN | |
| | TAVYYCARRDFHSSFDYW | | GHSFPLTFGPGTKVDIK | |
| | GQGTTVTVSS | | | |
| >12 | EVQLQQSGPELVKPGTSV | 27 | DIVLTQSPATLSVTPGNS | 28 |
| antibody | KISCKTSGYTFTEYSMHW | | VSLSCRASQSISDYLHWF | |
| | VKQSHGKSLEWFGGISPN | | QQKSHESPRLLIKYASQS | |
| | SGGTSYNQKFKGKATLTV | | ISGIPSRFSGSGSGSDFT | |
| | DKSSSTAYMELRSLTSED | | LSINSVEPEDVGVYYCQN | |
| | SAVYYCARRDFHSSFDYW | | GHSFPLTFGPGTKLELK | |
| | GQGTTLTVSS | | | |
| >12 | EVQLQQSGAEVKKPGSSV | 29 | DIVLTQSPATLSLSPGER | 30 |
| antibody_ | KVSCKTSGYTFTEYSMHW | | ATLSCRASQSISDYLHWF | |
| Humanized | VRQAPGKSLEWFGGISPN | | QQKPHESPRLLIKYASQS | |
| | SGGTSYNQKFKGRATLTV | | ISGIPARFSGSGSGSDFT | |
| | DKSSSTAYMELSSLRSED | | LTISSLEPEDFAVYYCQN | |
| | TAVYYCARRDFHSSFDYW | | GHSFPLTFGPGTKVDIK | |
| | GQGTTVTVSS | | | |

### Anti-CD38 Antibodies

Exemplary anti-CD38 antibody sequences are shown below. Table 6 provides illustrative heavy chain variable and light chain variable amino acid sequences for anti-CD38 antibodies according to the disclosure.

TABLE 6
Exemplary VH and VL region amino acid
sequences of anti-CD38 antibodies

| | | SEQ | | SEQ |
|---|---|---|---|---|
| | | ID | | ID |
| Ab ID | VH full length | NO | VL full length | NO |
| >Anti- | QVQLVQSGAEVKKPGAS | 38 | DIQMTQSPSSLSASVG | 39 |
| CD38 | VKVSCKASGYTFTSYWM | | DRVTITCRASENIYSF | |
| | QWVRQAPGQGLEWMGAI | | LAWYQQKPGKAPKLLI | |
| | YPGDGDTRYTQKFKGRV | | YNTKTLTEGVPSRFSG | |
| | TMTRDTSTSTVYMELSS | | SGSGTDFTLTISSLQP | |
| | LRSEDTAVYYCARERTT | | EDFATYYCQHHYGIPL | |
| | GAPRYFDVWGQGTLVTV | | TFGGGTKVEIK | |
| | SS | | | |

### Exemplary Anti-NKp46 And Anti-CD38 Bispecific Antibodies

In some embodiments, the anti-CD38/anti-NKp46 bispecific antibody includes a first heavy chain comprising CDRH1 comprising the amino acid sequence of SEQ ID NO: 17, a CDRH2 comprising the amino acid sequence of SEQ ID NO: 18, a CDRH3 comprising the amino acid sequence of SEQ ID NO: 19, a first light chain comprising a CDRL1 comprising the amino acid sequence of SEQ ID NO: 20, a CDRL2 comprising the amino acid sequence of SEQ ID NO: 21, and a CDRL3 comprising the amino acid sequence of SEQ ID NO: 22, a second heavy chain comprising CDRH1 comprising the amino acid sequence of SEQ ID NO: 32, a CDRH2 comprising the amino acid sequence of SEQ ID NO: 33, a CDRH3 comprising the amino acid sequence of SEQ ID NO: 34, and a second light chain comprising a CDRL1 comprising the amino acid sequence of SEQ ID NO: 35, a CDRL2 comprising the amino acid sequence of SEQ ID NO: 36, and a CDRL3 comprising the amino acid sequence of SEQ ID NO: 37.

In some embodiments, the anti-CD38/anti-NKp46 bispecific antibody includes a first heavy chain variable region comprising an amino acid sequence of SEQ ID NO: 25 or a sequence at least 80%, at least 90%, at least 95%, at least 96%, at least 97%, at least 98%, or at least 99% identical thereto, a first kappa light chain variable region comprising an amino acid sequence of SEQ ID NO: 26 or a sequence at least 80%, at least 90%, at least 95%, at least 96%, at least 97%, at least 98%, or at least 99% identical thereto, and a second heavy chain variable region comprising an amino acid sequence of SEQ ID NO: 38 or a sequence at least 80%, at least 90%, at least 95%, at least 96%, at least 97%, at least 98%, or at least 99% identical thereto; and a second kappa light chain variable region comprising an amino acid sequence of SEQ ID NO: 39 or a sequence at least 80%, at least 90%, at least 95%, at least 96%, at least 97%, at least 98%, or at least 99% identical thereto.

In some embodiments, the anti-CD38/anti-NKp46 bispecific antibody includes a fused heavy chain comprising an amino acid sequence of SEQ ID NO: 41 or a sequence at least 80%, at least 90%, at least 95%, at least 96%, at least 97%, at least 98%, or at least 99% identical thereto, a first kappa light chain comprising an amino acid sequence of SEQ ID NO: 31 or a sequence at least 80%, at least 90%, at least 95%, at least 96%, at least 97%, at least 98%, or at least 99% identical thereto and a second kappa light chain comprising an amino acid sequence of SEQ ID NO: 40 or a sequence at least 80%, at least 90%, at least 95%, at least 96%, at least 97%, at least 98%, or at least 99% identical thereto. Shown in bold in SEQ ID NOs: 41, 31, 40, and 46 is the signal sequence, which is not necessarily present in all embodiments.

>Fused-Heavy chain CD38/NKp46/huIgG1-L234A/L235A
(SEQ ID NO: 41)
MEWSGVFMFLLSVTAGVHSQVQLVQSGAEVKKPGASVKVSCKASGYTFTSYWM
QWVRQAPGQGLEWMGAIYPGDGDTRYTQKFKGRVTMTRDTSTSTVYMELSSLRS
EDTAVYYCARERTTGAPRYFDVWGQGTLVTVSSASTKGPSVFPLAPSSKSTSGGTA
ALGCLVKDYFPEPVTVSWNSGALTSGVHTFPAVLQSSGLYSLSSVVTVPSSSLGTQT

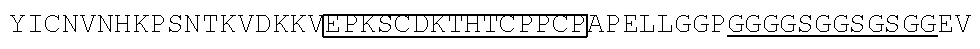

QLQQSGAEVKKPGSSVKVSCKTSGYTFTEYSMHWVRQAPGKSLEWFGGISPNSGG
TSYNQKFKGRATLTVDKSSSTAYMELSSLRSEDTAVYYCARRDFHSSFDYWGQGT
TVTVSSASTKGPSVFPLAPSSKSTSGGTAALGCLVKDYFPEPVTVSWNSGALTSGVH

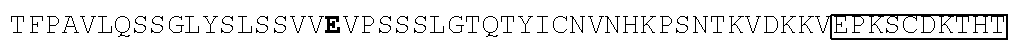

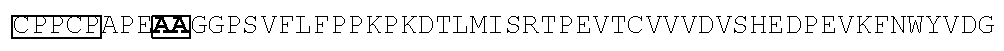

VEVHNAKTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSLSLSPGK
>Fused-Heavy chain CD38/NKp46/huIgG1-wt
(SEQ ID NO: 46)
MEWSGVFMFLLSVTAGVHSQVQLVQSGAEVKKPGASVKVSCKASGYTFTSYWM
QWVRQAPGQGLEWMGAIYPGDGDTRYTQKFKGRVTMTRDTSTSTVYMELSSLRS
EDTAVYYCARERTTGAPRYFDVWGQGTLVTVSSASTKGPSVFPLAPSSKSTSGGTA
ALGCLVKDYFPEPVTVSWNSGALTSGVHTFPAVLQSSGLYSLSSVVTVPSSSLGTQT
YICNVNHKPSNTKVDKKVEPKSCDKTHTCPPCPAPELLGGPGGGGSGGSGSGGEVQ
LQQSGAEVKKPGSSVKVSCKTSGYTFTEYSMHWVRQAPGKSLEWFGGISPNSGGT
SYNQKFKGRATLTVDKSSSTAYMELSSLRSEDTAVYYCARRDFHSSFDYWGQGTT
VTVSSASTKGPSVFPLAPSSKSTSGGTAALGCLVKDYFPEPVTVSWNSGALTSGVHT
FPAVLQSSGLYSLSSVVEVPSSSLGTQTYICNVNHKPSNTKVDKKVEPKSCDKTHTC
PPCPAPELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLSPGK
>light chain 1 anti-NkP46
(SEQ ID NO: 31)
MEWSGVFMFLLSVTAGVHSDIVMTQSPATLSLSPGERATLSCRASQSISDYLHWF
QQKPHESPRLLIKYASQSISGIPARFSGSGSGSDFTLTISSLEPEDFAVYYCQNGHSFP
LTFGPGTKVDIKRTVAAPAVFIFPPSDEQLKSGTASVVCLLKNFYPREAKVQWKVD
NALQSGNSQESVTEQDSKDSTYSLSSTLTLSKADYEKHKVYACEVTHQGLSSPVTK
SFNRGEC
>light chain 2 anti-CD38
(SEQ ID NO: 40)
MEWSGVFMFLLSVTAGVHSDIQMTQSPSSLSASVGDRVTITCRASENIYSFLAWY
QQKPGKAPKLLIYNTKTLTEGVPSRFSGSGSGTDFTLTISSLQPEDFATYYCQHHYGI
PLTFGGGTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVT
KSFNRGEC

The antibody can be expressed by a vector containing a DNA segment encoding the single chain antibody described above.

These can include vectors, liposomes, naked DNA, adjuvant-assisted DNA. gene gun, catheters, etc. Vectors include chemical conjugates such as described in WO 93/64701, which has targeting moiety (e.g., a ligand to a cellular surface receptor), and a nucleic acid binding moiety (e.g., polylysine), viral vector (e.g., a DNA or RNA viral vector), fusion proteins such as described in PCT/US 95/02140 (WO 95/22618) which is a fusion protein containing a target moiety (e.g., an antibody specific for a target cell) and a nucleic acid binding moiety (e.g., a protamine), plasmids, phage, etc. The vectors can be chromosomal, non-chromosomal or synthetic.

Preferred vectors include viral vectors, fusion proteins and chemical conjugates. Retroviral vectors include moloney murine leukemia viruses. DNA viral vectors are preferred. These vectors include pox vectors such as orthopox or avipox vectors, herpesvirus vectors such as a herpes simplex I virus (HSV) vector (see Geller, A. I. et al., J. Neurochem, 64:487 (1995); Lim, F., et al., in DNA Cloning: Mammalian Systems, D. Glover, Ed. (Oxford Univ. Press, Oxford England) (1995); Geller, A. I. et al., Proc Natl. Acad. Sci.: U.S.A. 90:7603 (1993); Geller, A. I., et al., Proc Natl. Acad. Sci USA 87:1149 (1990), Adenovirus Vectors (see LeGal LaSalle et al., Science, 259:988 (1993); Davidson, et al., Nat. Genet 3:219 (1993); Yang, et al., J. Virol. 69:2004 (1995) and Adeno-associated Virus Vectors (see Kaplitt, M. G. et al., Nat. Genet. 8:148 (1994).

Pox viral vectors introduce the gene into the cells cytoplasm. Avipox virus vectors result in only a short term expression of the nucleic acid. Adenovirus vectors, adeno-associated virus vectors and herpes simplex virus (HSV) vectors are preferred for introducing the nucleic acid into neural cells. The adenovirus vector results in a shorter term expression (about 2 months) than adeno-associated virus (about 4 months), which in turn is shorter than HSV vectors. The particular vector chosen will depend upon the target cell and the condition being treated. The introduction can be by standard techniques, e.g., infection, transfection, transduction or transformation. Examples of modes of gene transfer include e.g., naked DNA, CaPO₄ precipitation, DEAE dextran, electroporation, protoplast fusion, lipofection, cell microinjection, and viral vectors.

The vector can be employed to target essentially any desired target cell. For example, stereotaxic injection can be used to direct the vectors (e.g., adenovirus, HSV) to a desired location. Additionally, the particles can be delivered by intracerebroventricular (icv) infusion using a minipump infusion system, such as a SynchroMed Infusion System. A method based on bulk flow, termed convection, has also proven effective at delivering large molecules to extended areas of the brain and may be useful in delivering the vector to the target cell. (See Bobo et al., Proc. Natl. Acad. Sci. USA 91:2076-2080 (1994); Morrison et al., Am. J. Physiol. 266:292-305 (1994)). Other methods that can be used include catheters, intravenous, parenteral, intraperitoneal and subcutaneous injection, and oral or other known routes of administration.

Bispecific antibodies are antibodies that have binding specificities for at least two different antigens. In the present case, one of the binding specificities is for a target such as NKp46 or any fragment thereof. The second binding target is CD38 or any fragment thereof.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10:3655-3659 (1991).

Bispecific antibodies can be made using any of a variety of art-recognized techniques, including those disclosed in WO 2012/023053, the contents of which are hereby incorporated by reference in their entirety. The methods described in WO 2012/023053 generate bispecific antibodies that are identical in structure to a human immunoglobulin. This type of molecule is composed of two copies of a unique heavy chain polypeptide, a first light chain variable region fused to a constant Kappa domain and second light chain variable region fused to a constant Lambda domain. Each combining site displays a different antigen specificity to which both the heavy and light chain contribute. The light chain variable regions can be of the Lambda or Kappa family and are preferably fused to a Lambda and Kappa constant domains, respectively. This is preferred in order to avoid the generation of non-natural polypeptide junctions. However it is also possible to obtain bispecific antibodies of the invention by fusing a Kappa light chain variable domain to a constant Lambda domain for a first specificity and fusing a Lambda light chain variable domain to a constant Kappa domain for the second specificity. The bispecific antibodies described in WO 2012/023053 are referred to as IgGκλ antibodies or “κλ bodies,” a new fully human bispecific IgG format. This κλ-body format allows the affinity purification of a bispecific antibody that is undistinguishable from a standard IgG molecule with characteristics that are undistinguishable from a standard monoclonal antibody and, therefore, favorable as compared to previous formats.

An essential step of the method is the identification of two antibody Fv regions (each composed by a variable light chain and variable heavy chain domain) having different antigen specificities that share the same heavy chain variable domain. Numerous methods have been described for the generation of monoclonal antibodies and fragments thereof. (See, e.g., Antibodies: A Laboratory Manual, Harlow E, and Lane D, 1988, Cold Spring Harbor Laboratory Press, Cold Spring Harbor, NY, incorporated herein by reference). Fully human antibodies are antibody molecules in which the sequence of both the light chain and the heavy chain, including the CDRs 1 and 2, arise from human genes. The CDR3 region can be of human origin or designed by synthetic means. Such antibodies are termed “human antibodies”, or “fully human antibodies” herein. Human monoclonal antibodies can be prepared by using the trioma technique; the human B-cell hybridoma technique (see Kozbor, et al., 1983 Immunol Today 4: 72); and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

Monoclonal antibodies are generated, e.g., by immunizing an animal with a target antigen or an immunogenic fragment, derivative or variant thereof. Alternatively, the animal is immunized with cells transfected with a vector containing a nucleic acid molecule encoding the target antigen, such that the target antigen is expressed and associated with the surface of the transfected cells. A variety of techniques are well-known in the art for producing xenogenic non-human animals. For example, see U.S. Pat. Nos. 6,075,181 and 6,150,584, which is hereby incorporated by reference in its entirety.

Alternatively, the antibodies are obtained by screening a library that contains antibody or antigen binding domain sequences for binding to the target antigen. This library is prepared, e.g., in bacteriophage as protein or peptide fusions to a bacteriophage coat protein that is expressed on the surface of assembled phage particles and the encoding DNA sequences contained within the phage particles (i.e., “phage displayed library”).

Hybridomas resulting from myeloma/B cell fusions are then screened for reactivity to the target antigen. Monoclonal antibodies are prepared, for example, using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

Although not strictly impossible, the serendipitous identification of different antibodies having the same heavy chain variable domain but directed against different antigens is highly unlikely. Indeed, in most cases the heavy chain contributes largely to the antigen binding surface and is also the most variable in sequence. In particular the CDR3 on the heavy chain is the most diverse CDR in sequence, length and structure. Thus, two antibodies specific for different antigens will almost invariably carry different heavy chain variable domains.

The methods disclosed in co-pending application WO 2012/023053 overcomes this limitation and greatly facilitates the isolation of antibodies having the same heavy chain variable domain by the use of antibody libraries in which the heavy chain variable domain is the same for all the library members and thus the diversity is confined to the light chain variable domain. Such libraries are described, for example, in co-pending applications WO 2010/135558 and WO 2011/084255, each of which is hereby incorporated by reference in its entirety. However, as the light chain variable domain is expressed in conjunction with the heavy variable domain, both domains can contribute to antigen binding. To further facilitate the process, antibody libraries containing the same heavy chain variable domain and either a diversity of Lambda variable light chains or Kappa variable light chains can be used in parallel for in vitro selection of antibodies against different antigens. This approach enables the identification of two antibodies having a common heavy chain but one carrying a Lambda light chain variable domain and the other a Kappa light chain variable domain that can be used as building blocks for the generation of a bispecific antibody in the full immunoglobulin format of the invention.

The common heavy chain and two different light chains are co-expressed into a single cell to allow for the assembly of a bispecific antibody of the invention. If all the polypeptides get expressed at the same level and get assembled equally well to form an immunoglobulin molecule then the ratio of monospecific (same light chains) and bispecific (two different light chains) should be 50%. However, it is likely that different light chains are expressed at different levels and/or do not assemble with the same efficiency. Therefore, a means to modulate the relative expression of the different polypeptides is used to compensate for their intrinsic expression characteristics or different propensities to assemble with the common heavy chain. This modulation can be achieved via promoter strength, the use of internal ribosome entry sites (IRES) featuring different efficiencies or other types of regulatory elements that can act at transcriptional or translational levels as well as acting on mRNA stability. Different promoters of different strength could include CMV (Immediate-early Cytomegalovirus virus promoter); EF1-1α (Human elongation factor 1α-subunit promoter); Ubc (Human ubiquitin C promoter); SV40 (Simian virus 40 promoter). Different IRES have also been described from mammalian and viral origin. (See e.g., Hellen C U and Sarnow P. Genes Dev 2001 15: 1593-612). These IRES can greatly differ in their length and ribosome recruiting efficiency. Furthermore, it is possible to further tune the activity by introducing multiple copies of an IRES (Stephen et al. 2000 Proc Natl Acad Sci USA 97: 1536-1541). The modulation of the expression can also be achieved by multiple sequential transfections of cells to increase the copy number of individual genes expressing one or the other light chain and thus modify their relative expressions. The Examples provided herein demonstrate that controlling the relative expression of the different chains is critical for maximizing the assembly and overall yield of the bispecific antibody.

The co-expression of the heavy chain and two light chains generates a mixture of three different antibodies into the cell culture supernatant: two monospecific bivalent antibodies and one bispecific bivalent antibody. The latter has to be purified from the mixture to obtain the molecule of interest. The method described herein greatly facilitates this purification procedure by the use of affinity chromatography media that specifically interact with the Kappa or Lambda light chain constant domains such as the CaptureSelect Fab Kappa and CaptureSelect Fab Lambda affinity matrices (BAC BV, Holland). This multi-step affinity chromatography purification approach is efficient and generally applicable to antibodies of the invention. This is in sharp contrast to specific purification methods that have to be developed and optimized for each bispecific antibodies derived from quadromas or other cell lines expressing antibody mixtures. Indeed, if the biochemical characteristics of the different antibodies in the mixtures are similar, their separation using standard chromatography technique such as ion exchange chromatography can be challenging or not possible at all.

Other suitable purification methods include those disclosed in co-pending application PCT/IB2012/003028, filed on Oct. 19, 2012, published as WO2013/088259, the contents of which are hereby incorporated by reference in their entirety.

In other embodiments of producing bispecific antibodies, antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface includes at least a part of the CH3 region of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Techniques for generating bispecific antibodies from antibody fragments have been described in the literature. For example, bispecific antibodies can be prepared using chemical linkage. The bispecific antibodies produced can be used as agents for the selective immobilization of enzymes.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (VH) connected to a light-chain variable domain (VL) by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g., CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction.

### Conjugates

Heteroconjugate antibodies are also within the scope of the present invention. Heteroconjugate antibodies are composed of two covalently joined antibodies. Such antibodies have, for example, been proposed to target immune system cells to unwanted cells (see U.S. Pat. No. 4,676,980), and for treatment of HIV infection (see WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

The invention also pertains to immunoconjugates comprising an antibody conjugated to a cytotoxic agent such as a toxin (e.g., an enzymatically active toxin of bacterial, fungal, plant, or animal origin, or fragments thereof), or a radioactive isotope (i.e., a radioconjugate).

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. (See WO94/11026).

Those of ordinary skill in the art will recognize that a large variety of possible moieties can be coupled to the resultant antibodies of the invention. (See, for example, “Conjugate Vaccines”, Contributions to Microbiology and Immunology, J. M. Cruse and R. E. Lewis, Jr (eds), Carger Press, New York, (1989), the entire contents of which are incorporated herein by reference).

Coupling may be accomplished by any chemical reaction that will bind the two molecules so long as the antibody and the other moiety retain their respective activities. This linkage can include many chemical mechanisms, for instance covalent binding, affinity binding, intercalation, coordinate binding and complexation. The preferred binding is, however, covalent binding. Covalent binding can be achieved either by direct condensation of existing side chains or by the incorporation of external bridging molecules. Many bivalent or polyvalent linking agents are useful in coupling protein molecules, such as the antibodies of the present invention, to other molecules. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents. (See Killen and Lindstrom, Jour. Immun. 133:1335-2549 (1984); Jansen et al., Immunological Reviews 62:185-216 (1982); and Vitetta et al., Science 238:1098 (1987).

Preferred linkers are described in the literature. (See, for example, Ramakrishnan, S. et al., Cancer Res. 44:201-208 (1984) describing use of MBS (M-maleimidobenzoyl-N-hydroxysuccinimide ester). See also, U.S. Pat. No. 5,030,719, describing use of halogenated acetyl hydrazide derivative coupled to an antibody by way of an oligopeptide linker. Particularly preferred linkers include: (i) EDC (1-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido]hexanoate (Pierce Chem. Co., Cat #21651G); (iv) Sulfo-LC-SPDP (sulfosuccinimidyl 6 [3-(2-pyridyldithio)-propianamide] hexanoate (Pierce Chem. Co. Cat. #2165-G); and (v) sulfo-NHS (N-hydroxysulfo-succinimide: Pierce Chem. Co., Cat. #24510) conjugated to EDC.

The linkers described above contain components that have different attributes, thus leading to conjugates with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form conjugates with increased stability. Disulfide linkages, are in general, less stable than other linkages because the disulfide linkage is cleaved in vitro, resulting in less conjugate available. Sulfo-NHS, in particular, can enhance the stability of carbodimide couplings. Carbodimide couplings (such as EDC) when used in conjunction with sulfo-NHS, forms esters that are more resistant to hydrolysis than the carbodimide coupling reaction alone.

### Fc-Modified Antibodies

The bispecific antibodies of the invention can be of different Isotypes and their Fc portion can be modified in order to alter the bind properties to different Fc receptors and in this way modify the effectors functions of the antibody as well as it pharmacokinetic properties. Numerous methods for the modification of the Fc portion have been described and are applicable to antibodies of the invention. (see for example Strohl, WR Curr Opin Biotechnol 2009 (6):685-91; U.S. Pat. No. 6,528,624; PCT/US2009/0191199 filed Jan. 9, 2009). The methods of the invention can also be used to generate bispecific antibodies and antibody mixtures in a F(ab′)2 format that lacks the Fc portion.

It can be desirable to modify the antibody of the invention with respect to effector function, so as to enhance, e.g., the effectiveness of the antibody in treating cancer and/or other diseases and disorders associated with aberrant NKp46 and/or CD38 expression and/or activity. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). (See Caron et al., J. Exp Med., 176: 1191-1195 (1992) and Shopes, J. Immunol., 148: 2918-2922 (1992)). Alternatively, an antibody can be engineered that has dual Fc regions and can thereby have enhanced complement lysis and ADCC capabilities. (See Stevenson et al., Anti-Cancer Drug Design, 3: 219-230 (1989)).

Mutations enhancing the effector function of antibodies are known in the art, see, e.g., Saunders et al., 2019; Front. Immunol. 10:1296 and Wang et al., 2018, Protein Cell 9(1): 63-73, each of which is incorporated herein by reference in its entirety for examples of Fc modifications that may be introduced in the antibodies described herein. Unless otherwise indicated, the Fc mutations in this section are described with reference to the Kabat numbering scheme for immunoglobulins.

For example, Lys326Trp/Glu333Ser and Ser267Glu/His268Phe/Ser324Thr Fc mutants show decreased ADCC, while Lys326Trp/Glu333Ser, Lys326Ala/Glu333Ala, Lys326Met/Glu333Ser, Cys221Asp/Asp222Cys, Ser267Glu, His268Phe, and Ser324Thr, and Glu345Arg Fc mutants show increased C1q binding. On the other hand, S239D/1332E and S239D/I332E/A330L Fc mutants are associated with increased ADCC activity.

Other mutations in the Fc region can improve antibody circulation half-life, for example, Arg435His, Met252Tyr/Ser254Thr/Thr256Glu (“YTE”), Met428Leu/Asn434Ser, and Thr252Leu/Thr253Ser/Thr254Phe mutants show extended half-life compared to the unmutated versions.

In other embodiments, an Fc mutation may results in the loss of an effector function, for example, ablation of Fc receptor binding. Examples of Fc mutants that decrease binding to one or more Fc receptors include Leu235Glu, Leu234Ala/Leu235Ala (“LALA”), Ser228Pro/Leu235Glu, Leu234Ala/Leu235Ala/Pro329Gly, Pro331Ser/Leu234Glu/Leu235Phe, Asp265Ala, and Ala330Leu.

Furthermore, binding to Fc receptors may be achieved by glycoengineering. Glycoengineering may involve the modification of the glycosylation site at amino acid N297 of the CH2 domain of immunoglobulin. Alternatively or additionally, recombinantly produced antibodies may be post-translationally modified by exposing the cell culture producing the antibody to glycosylation inhibitors. Post-translational modifications glycosylation, carboxylation, deamidation, oxidation, hydroxylation, O-sulfation, amidation, glycylation, glycation, alkylation, acylation, acetylation, phosphorylation, biotinylation, formylation, lipidation, iodination, prenylation, oxidation, palmitoylation, phosphatidylinositolation, phosphopantetheinylation, sialylation, and selenoylation, C-terminal Lysine removal. Illustrative glycosylation inhibitors are described I, for example, U.S. Pat. No. 9,868,973, which is incorporated herein by reference in its entirety for examples of glycosylation inhibitors that may be used in the production of the antibodies described herein.

### NK Cells

In some embodiments, the present disclosure provides methods for immunotherapy comprising administering an effective amount of the bispecific antibody and immune cells of the present disclosure (e.g. NK cells).

NK cells are a subpopulation of lymphocytes that have spontaneous cytotoxicity against a variety of tumor cells, virus-infected cells, and some normal cells in the bone marrow and thymus. NK cells are critical effectors of the early innate immune response toward transformed and virus-infected cells. NK cells constitute about 10% of the lymphocytes in human peripheral blood. When lymphocytes are cultured in the presence of IL-2, strong cytotoxic reactivity develops. NK cells are effector cells known as large granular lymphocytes because of their larger size and the presence of characteristic azurophilic granules in their cytoplasm. NK cells differentiate and mature in the bone marrow, lymph nodes, spleen, tonsils, and thymus. NK cells can be detected by specific surface markers, such as CD16, CD56, and CD8 in humans. NK cells do not express T cell antigen receptors, the pan T marker CD3, or surface immunoglobulin B cell receptors.

Stimulation of NK cells is achieved through a cross-talk of signals derived from cell surface activating and inhibitory receptors. The activation status of NK cells is regulated by a balance of intracellular signals received from an array of germ-line-encoded activating and inhibitory receptors (Campbell, 2006). When NK cells encounter an abnormal cell (e.g., tumor or virus-infected cell) and activating signals predominate, the NK cells can rapidly induce apoptosis of the target cell through directed secretion of cytolytic granules containing perform and granzymes or engagement of death domain-containing receptors.

Activated NK cells can also secrete type I cytokines, such as interferon-γ, tumor necrosis factor-a and granulocyte-macrophage colony-stimulating factor (GM-CSF), which activate both innate and adaptive immune cells as well as other cytokines and chemokines (Wu et al., 2003). Production of these soluble factors by NK cells in early innate immune responses significantly influences the recruitment and function of other hematopoietic cells. Also, through physical contacts and production of cytokines, NK cells are central players in a regulatory crosstalk network with dendritic cells and neutrophils to promote or restrain immune responses.

In certain embodiments, NK cells are derived from human peripheral blood mononuclear cells (PBMC), unstimulated leukapheresis products (PBSC), human embryonic stem cells (hESCs), induced pluripotent stem cells (iPSCs), mesenchymal stem cells (MSCs), hematopoietic stem cells (HSCs), bone marrow, CD34+ cells or umbilical cord blood (CB) by methods well known in the art. In some embodiments, NK cells are isolated from PBMC. In some embodiments, umbilical CB is used to derive NK cells. In certain aspects, the NK cells are isolated and expanded by the previously described method of ex vivo expansion of NK cells (Shah et al, 2013). In this method, CB mononuclear cells are isolated by ficoll density gradient centrifugation and cultured in a bioreactor with IL-2 and artificial antigen presenting cells (aAPCs). After 7 days, the cell culture is depleted of any cells expressing CD3 and re-cultured for an additional 7 days. The cells are again CD3-depleted and characterized to determine the percentage of CD56+/CD3+ cells or NK cells. In some embodiments, umbilical CB is used to derive NK cells by the isolation of CD34+ cells and differentiation into CD56+/CD3+ cells by culturing in medium containing SCF, IL-7, IL-15, and IL-2.

Exemplary methods of isolating and deriving NK cells include but are not limited to those described in U.S. Pat. No. 9,260,696. Exemplary methods of generating iPSC-NK cells are also described in Zhu and Kaufman, Mol. Bio, 2019, Yang et. Al., Mol Ther: Meth Clin Dev, 2020 and Moseman et. al., 2020.

### Methods of Use

It will be appreciated that administration of therapeutic entities in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company, Easton, PA (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2):1-60 (2000), Charman W N “Lipids, lipophilic drugs, and oral drug delivery-some emerging concepts.” J Pharm Sci. 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

Therapeutic formulations of the invention, which include an antibody of the invention, are used to treat or alleviate a symptom associated with a cancer, such as, by way of non-limiting example, leukemias, lymphomas, breast cancer, colon cancer, ovarian cancer, bladder cancer, prostate cancer, glioma, lung & bronchial cancer, colorectal cancer, pancreatic cancer, esophageal cancer, liver cancer, urinary bladder cancer, kidney and renal pelvis cancer, oral cavity & pharynx cancer, uterine corpus cancer, and/or melanoma The present invention also provides methods of treating or alleviating a symptom associated with a cancer. A therapeutic regimen is carried out by identifying a subject, e.g., a human patient suffering from (or at risk of developing) a cancer, using standard methods.

Efficaciousness of treatment is determined in association with any known method for diagnosing or treating the particular immune-related disorder. Alleviation of one or more symptoms of the immune-related disorder indicates that the antibody confers a clinical benefit.

Methods for the screening of antibodies that possess the desired specificity include, but are not limited to, enzyme linked immunosorbent assay (ELISA) and other immunologically mediated techniques known within the art.

Antibodies directed against a target such as NKp46, CD38, or a combination thereof (or a fragment thereof), may be used in methods known within the art relating to the localization and/or quantitation of these targets, e.g., for use in measuring levels of these targets within appropriate physiological samples, for use in diagnostic methods, for use in imaging the protein, and the like). In a given embodiment, antibodies specific any of these targets, or derivative, fragment, analog or homolog thereof, that contain the antibody derived antigen binding domain, are utilized as pharmacologically active compounds (referred to hereinafter as “Therapeutics”).

An antibody of the invention can be used to isolate a particular target using standard techniques, such as immunoaffinity, chromatography or immunoprecipitation. Antibodies of the invention (or a fragment thereof) can be used diagnostically to monitor protein levels in tissue as part of a clinical testing procedure, e.g., to determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibodies of the invention, including polyclonal, monoclonal, humanized and fully human antibodies, may be used as therapeutic agents. Such agents will generally be employed to treat or prevent a disease or pathology associated with aberrant expression or activation of a given target in a subject. An antibody preparation, preferably one having high specificity and high affinity for its target antigen, is administered to the subject and will generally have an effect due to its binding with the target. Administration of the antibody may abrogate or inhibit or interfere with the signaling function of the target. Administration of the antibody may abrogate or inhibit or interfere with the binding of the target with an endogenous ligand to which it naturally binds. For example, the antibody binds to the target and neutralizes or otherwise inhibits the interaction between CD38 and its endogenous ligand. For example, the antibody binds to the target and neutralizes or otherwise inhibits the interaction between NKp46 and its endogenous ligand.

A therapeutically effective amount of an antibody of the invention relates generally to the amount needed to achieve a therapeutic objective. As noted above, this may be a binding interaction between the antibody and its target antigen that, in certain cases, interferes with the functioning of the target. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from twice daily to once a week.

Antibodies or a fragment thereof of the invention can be administered for the treatment of a variety of diseases and disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. (See, e.g., Marasco et al., Proc. Natl. Acad. Sci. USA, 90: 7889-7893 (1993)). The formulation can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

An antibody according to the invention can be used as an agent for detecting the presence of a given target (or a protein fragment thereof) in a sample. In some embodiments, the antibody contains a detectable label. Antibodies are polyclonal, or more preferably, monoclonal. An intact antibody, or a fragment thereof (e.g., F_{ab}, scFv, or F_{(ab)2}) is used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, N J, 1995; “Immunoassay”, E. Diamandis and T. Christopoulus, Academic Press, Inc., San Diego, C A, 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti-analyte protein antibody. For example, the antibody can be labeled with a radioactive marker whose presence and location in a subject can be detected by standard imaging techniques.

### Diseases and Disorders

In some embodiments, a medical disease or disorder is treated by transfer of an immune cell population that elicits an immune response. In certain embodiments of the present disclosure, cancer or infection is treated by transfer of an immune cell population that elicits an immune response. Provided herein are methods for treating or delaying progression of cancer in an individual comprising administering to the individual an effective amount an antigen-specific cell therapy. The present methods may be applied for the treatment of immune disorders, solid cancers, hematologic cancers, and viral infections.

Tumors for which the present treatment methods are useful include any malignant cell type, such as those found in a solid tumor or a hematological tumor. Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); myelodysplastic syndrome (MDS); chronic myeloblasts leukemia; diffuse large B-cell lymphoma (DLBCL); peripheral T-cell lymphoma (PTCL); or anaplastic large cell lymphoma (ALCL). In some embodiments, the cancer is multiple myeloma. In some embodiments, the cancer is lymphoma. In some embodiments, the cancer is AML.

In certain embodiments of the present disclosure, immune cells (e.g. NK cells) are delivered to an individual in need thereof, such as an individual that has cancer or an infection. The cells then enhance the individual's immune system to attack or directly attack the respective cancer or pathogenic cells. In some cases, the individual is provided with one or more doses of the immune cells. In cases where the individual is provided with two or more doses of the immune cells, the duration between the administrations should be sufficient to allow time for propagation in the individual, and in specific embodiments the duration between doses is 1, 2, 3, 4, 5, 6, 7, 8, 9, 10, 11, or 12 or more weeks.

Certain embodiments of the present disclosure provide methods for treating or preventing an immune-mediated disorder. In one embodiment, the subject has an autoimmune disease. Non-limiting examples of autoimmune diseases include: alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac spate-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Meniere's disease, mixed connective tissue disease, multiple sclerosis, type 1 or immune-mediated diabetes mellitus, myasthenia gravis, nephrotic syndrome (such as minimal change disease, focal glomerulosclerosis, or mebranous nephropathy), pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomenon, Reiter's syndrome, Rheumatoid arthritis, sarcoidosis, scleroderma, Sjogren's syndrome, stiff-man syndrome, systemic lupus erythematosus, lupus erythematosus, ulcerative colitis, uveitis, vasculitides (such as polyarteritis nodosa, takayasu arteritis, temporal arteritis/giant cell arteritis, or dermatitis herpetiformis vasculitis), vitiligo, and Wegener's granulomatosis. Thus, some examples of an autoimmune disease that can be treated using the methods disclosed herein include, but are not limited to, multiple sclerosis, rheumatoid arthritis, systemic lupus erythematosis, type I diabetes mellitus, Crohn's disease; ulcerative colitis, myasthenia gravis, glomerulonephritis, ankylosing spondylitis, vasculitis, or psoriasis. The subject can also have an allergic disorder such as Asthma.

In yet another embodiment, the subject is the recipient of a transplanted organ or stem cells and immune cells are used to prevent and/or treat rejection. In particular embodiments, the subject has or is at risk of developing graft versus host disease. GVHD is a possible complication of any transplant that uses or contains stem cells from either a related or an unrelated donor. There are two kinds of GVHD, acute and chronic. Acute GVHD appears within the first three months following transplantation. Signs of acute GVHD include a reddish skin rash on the hands and feet that may spread and become more severe, with peeling or blistering skin. Acute GVHD can also affect the stomach and intestines, in which case cramping, nausea, and diarrhea are present. Yellowing of the skin and eyes (jaundice) indicates that acute GVHD has affected the liver. Chronic GVHD is ranked based on its severity: stage/grade 1 is mild; stage/grade 4 is severe. Chronic GVHD develops three months or later following transplantation. The symptoms of chronic GVHD are similar to those of acute GVHD, but in addition, chronic GVHD may also affect the mucous glands in the eyes, salivary glands in the mouth, and glands that lubricate the stomach lining and intestines. Any of the populations of immune cells disclosed herein can be utilized. Examples of a transplanted organ include a solid organ transplant, such as kidney, liver, skin, pancreas, lung and/or heart, or a cellular transplant such as islets, hepatocytes, myoblasts, bone marrow, or hematopoietic or other stem cells. The transplant can be a composite transplant, such as tissues of the face. Immune cells can be administered prior to transplantation, concurrently with transplantation, or following transplantation. In some embodiments, the immune cells are administered prior to the transplant, such as at least 1 hour, at least 12 hours, at least 1 day, at least 2 days, at least 3 days, at least 4 days, at least 5 days, at least 6 days, at least 1 week, at least 2 weeks, at least 3 weeks, at least 4 weeks, or at least 1 month prior to the transplant. In one specific, non-limiting example, administration of the therapeutically effective amount of immune cells occurs 3-5 days prior to transplantation.

In some embodiments, the subject can be administered nonmyeloablative lymphodepleting chemotherapy prior to the immune cell therapy. The nonmyeloablative lymphodepleting chemotherapy can be any suitable such therapy, which can be administered by any suitable route. The nonmyeloablative lymphodepleting chemotherapy can comprise, for example, the administration of cyclophosphamide and fludarabine. An exemplary route of administering cyclophosphamide and fludarabine is intravenously. Likewise, any suitable dose of cyclophosphamide and fludarabine can be administered. In particular aspects, around 60 mg/kg of cyclophosphamide is administered for two days after which around 25 mg/m² fludarabine is administered for five days.

In some embodiments, the subject can be administered nonmyeloablative lymphodepleting immunotherapy prior to the immune cell therapy. The nonmyeloablative lymphodepleting immunotherapy can be any suitable such therapy, which can be administered by any suitable route. The nonmyeloablative lymphodepleting immunotherapy can comprise, for example, the administration of an anti-CD52 agent or anti-CD20 agent. In some embodiments, the lymphodepleting immunotherapy is an anti-CD52 antibody. In some embodiments, the anti-CD52 antibody is alemtuzumab. In some embodiments, the lymphodepleting immunotherapy is an anti-CD20 antibody. Exemplary anti-CD20 antibodies include, but are not limited to rituximab, ofatumumab, ocrelizumab, obinutuzumab, ibritumomab or iodine i131 tositumomab. An exemplary route of administering anti-CD52 agent or anti-CD20 agent is intravenously. Likewise, any suitable dose of anti-CD52 agent or anti-agent can be administered.

In certain embodiments, a growth factor that promotes the growth and activation of the immune cells is administered to the subject either concomitantly with the immune cells or subsequently to the immune cells. The immune cell growth factor can be any suitable growth factor that promotes the growth and activation of the immune cells. Examples of suitable immune cell growth factors include interleukin (IL)-2, IL-7, IL-15, and IL-12, which can be used alone or in various combinations, such as IL-2 and IL-7, IL-2 and IL-15, IL-7 and IL-15, IL-2, IL-7 and IL-15, IL-12 and IL-7, IL-12 and IL-15, or IL-12 and IL2.

Therapeutically effective amounts of immune cells can be administered by a number of routes, including parenteral administration, for example, intravenous, intraperitoneal, intramuscular, intrastemal, or intraarticular injection, or infusion.

The therapeutically effective amount of immune cells for use in adoptive cell therapy is that amount that achieves a desired effect in a subject being treated. For instance, this can be the amount of immune cells necessary to inhibit advancement, or to cause regression of an autoimmune or alloimmune disease, or which is capable of relieving symptoms caused by an autoimmune disease, such as pain and inflammation. It can be the amount necessary to relieve symptoms associated with inflammation, such as pain, edema and elevated temperature. It can also be the amount necessary to diminish or prevent rejection of a transplanted organ.

The immune cell population can be administered in treatment regimens consistent with the disease, for example a single or a few doses over one to several weeks to ameliorate a disease state or periodic doses over an extended time to inhibit disease progression and prevent disease recurrence. The precise dose to be employed in the formulation will also depend on the route of administration, and the seriousness of the disease or disorder. The therapeutically effective amount of immune cells will be dependent on the subject being treated, the severity and type of the affliction, and the manner of administration. In some embodiments, doses that could be used in the treatment of human subjects range from at least 3.8×10⁴, at least 3.8×10⁵, at least 3.8×10⁶, at least 3.8×10⁷, at least 3.8×10⁸, at least 3.8×10⁹, or at least 3.8×10¹⁰ immune cells/m². In a certain embodiment, the dose used in the treatment of human subjects ranges from about 3.8×10⁹ to about 3.8×10¹⁰ immune cells/m². In additional embodiments, a therapeutically effective amount of immune cells can vary from about 5×10⁶ cells per kg body weight to about 7.5×10⁸ cells per kg body weight, such as from about 2×10⁷ cells to about 5×10⁸ cells per kg body weight, or from about 5×10⁷ cells to about 2×10⁸ cells per kg body weight, or from about 5×10⁶ cells per kg body weight to about 1×10⁷ cells per kg body weight. In some embodiments, a therapeutically effective amount of immune cells ranges from about 1×10⁵ cells per kg body weight to about 10×10⁹ cells per kg body weight. The exact amount of immune cells is readily determined by one of skill in the art based on the age, weight, sex, and physiological condition of the subject. Effective doses can be extrapolated from dose-response curves derived from in vitro or animal model test systems.

The immune cells may be administered in combination with one or more other therapeutic agents for the treatment of the immune-mediated disorder. Combination therapies can include, but are not limited to, one or more anti-microbial agents (for example, antibiotics, anti-viral agents and anti-fungal agents), anti-tumor agents (for example, fluorouracil, methotrexate, paclitaxel, fludarabine, etoposide, doxorubicin, or vincristine), immune-depleting agents (for example, fludarabine, etoposide, doxorubicin, or vincristine), immunosuppressive agents (for example, azathioprine, or glucocorticoids, such as dexamethasone or prednisone), anti-inflammatory agents (for example, glucocorticoids such as hydrocortisone, dexamethasone or prednisone, or non-steroidal anti-inflammatory agents such as acetyls alicylic acid, ibuprofen or naproxen sodium), cytokine antagonists (for example, anti-TNF and anti-IL-6), cytokines (for example, interleukin-10 or transforming growth factor-beta), hormones (for example, estrogen), or a vaccine. In addition, immunosuppressive or tolerogenic agents including but not limited to calcineurin inhibitors (e.g., cyclosporin and tacrolimus); mTOR inhibitors (e.g., Rapamycin); mycophenolate mofetil, antibodies (e.g., recognizing CD3, CD4, CD40, CD154, CD45, IVIG, or B cells); chemotherapeutic agents (e.g., Methotrexate, Treosulfan, Busulfan); irradiation; or chemokines, interleukins or their inhibitors (e.g., BAFF, IL-2, anti-IL-2R, IL-4, JAK kinase inhibitors) can be administered. Such additional pharmaceutical agents can be administered before, during, or after administration of the immune cells, depending on the desired effect.

This administration of the cells and the agent can be by the same route or by different routes, and either at the same site or at a different site.

### Combination Therapies

In some embodiments, the compositions and methods of the present embodiments involve an immune cell population in combination with at least one additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

An immune cell therapy may be administered before, during, after, or in various combinations relative to an additional cancer therapy, such as immune checkpoint therapy. The administrations may be in intervals ranging from concurrently to minutes to days to weeks. In embodiments where the immune cell therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below an immune cell therapy is “A” and an anti-cancer therapy is “B”:
A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

### Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBT-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, decitabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above. In some embodiments, azacitidine is administered at 75 mgs/m² subcutaneously.

### Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

### Immunotherapy

The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells.

Antibody-drug conjugates have emerged as a breakthrough approach to the development of cancer therapeutics. Cancer is one of the leading causes of deaths in the world. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al, 1998); cytokine therapy, e.g., interferons α, β, and γ, IL-1, GM-CSF, and TNF (Bukowski et al, 1998; Davidson et al, 1998; Hellstrand et al, 1998); gene therapy, e.g., TNF, IL-1, IL-2, and p53 (Qin et al, 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al, 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Application No. US20140294898, US2014022021, and US20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO*, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD 152. The complete cDNA sequence of human CTLA-4 has the Genbank accession number L15006. CTLA-4 is found on the surface of T cells and acts as an “off switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al. (1998) Proc Natl Acad Sci USA 95(17): 10067-10071; Camacho et al. (2004)/Clin Oncology 22(145): Abstract No. 2505 (antibody CP-675206); and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

Examples of immunotherapies for use in treatment of kidney cancer or renal cell cancer include, but are not limited to Afinitor (Everolimus), Afinitor Disperz (Everolimus), Aldesleukin, Avastin (Bevacizumab), Avelumab, Axitinib, Bavencio (Avelumab), Bevacizumab, Cabometyx (Cabozantinib-S-Malate), Cabozantinib-S-Malate, Everolimus, IL-2 (Aldesleukin), Inlyta (Axitinib), Interleukin-2 (Aldesleukin), Ipilimumab, Keytruda (Pembrolizumab), Lenvatinib Mesylate, Lenvima (Lenvatinib Mesylate), Mvasi (Bevacizumab), Nexavar (Sorafenib Tosylate), Nivolumab, Opdivo (Nivolumab), Pazopanib, Hydrochloride, Pembrolizumab, Proleukin (Aldesleukin), Sorafenib Tosylate, Sunitinib Malate, Sutent (Sunitinib Malate), Temsirolimus, Torisel (Temsirolimus), Votrient (Pazopanib Hydrochloride), Yervoy (Ipilimumab).

Examples of immunotherapies for use in treatment of Acute Myeloid Leukemia (AML) include, but are not limited to Azacitidine, Arsenic Trioxide, Cerubidine (Daunorubicin Hydrochloride), Cyclophosphamide, Cytarabine, Daunorubicin Hydrochloride, Daunorubicin Hydrochloride and Cytarabine Liposome, Daurismo (Glasdegib Maleate), Dexamethasone, Doxorubicin Hydrochloride, Enasidenib Mesylate, Gemtuzumab Ozogamicin, Gilteritinib Fumarate, Glasdegib Maleate, Idamycin PFS (Idarubicin Hydrochloride), Idarubicin Hydrochloride, Idhifa (Enasidenib Mesylate), Ivosidenib, Midostaurin, Mitoxantrone Hydrochloride, Mylotarg (Gemtuzumab Ozogamicin), Rubidomycin (Daunorubicin Hydrochloride), Rydapt (Midostaurin), Tabloid (Thioguanine), Thioguanine, Tibsovo (Ivosidenib), Trisenox (Arsenic Trioxide), Venclexta (Venetoclax), Venetoclax, Vincristine Sulfate, Vyxeos (Daunorubicin Hydrochloride and Cytarabine Liposome), Xospata (Gilteritinib Fumarate).

### Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### Pharmaceutical Compositions

The antibodies of the invention (also referred to herein as “active compounds”), and derivatives, fragments, analogs and homologs thereof, can be incorporated into pharmaceutical compositions suitable for administration. Such compositions typically comprise the antibody and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

Also provided herein are pharmaceutical compositions and formulations comprising immune cells (e.g., NK cells) and a pharmaceutically acceptable carrier.

In some embodiments, a pharmaceutical composition comprises a dose ranging from about 1×10⁵ NK cells to about 1×10⁹ NK cells. In some embodiments, the dose is about 1×10⁵, 1×10⁶, 1×10⁷, 1×10⁸ or 1×10⁹ NK cells. In some embodiments, a pharmaceutical composition comprises a dose ranging from about 5×10⁵ NK cells to about 10×10¹² NK cells

In some embodiments, a pharmaceutical composition is cryopreserved.

Pharmaceutical compositions and formulations as described herein can be prepared by mixing the active ingredients (such as an antibody or a polypeptide) having the desired degree of purity with one or more optional pharmaceutically acceptable carriers (Remington's Pharmaceutical Sciences 22^{nd} edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn— protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

### Articles of Manufacture or Kits

An article of manufacture or a kit is provided comprising the bispecific antibodies and immune cells is also provided herein. The article of manufacture or kit can further comprise a package insert comprising instructions for using the immune cells to treat or delay progression of cancer in an individual or to enhance immune function of an individual having cancer. Any of the antigen-specific immune cells described herein may be included in the article of manufacture or kits. Suitable containers include, for example, bottles, vials, bags and syringes. The container may be formed from a variety of materials such as glass, plastic (such as polyvinyl chloride or poly olefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

### Examples

### Example 1: Generation of Mouse Monoclonal Antibodies that Bind to Human NKp46

### Binding of Mouse Anti-NKp46 Monoclonal Antibodies to NKp46 Expressing Cells

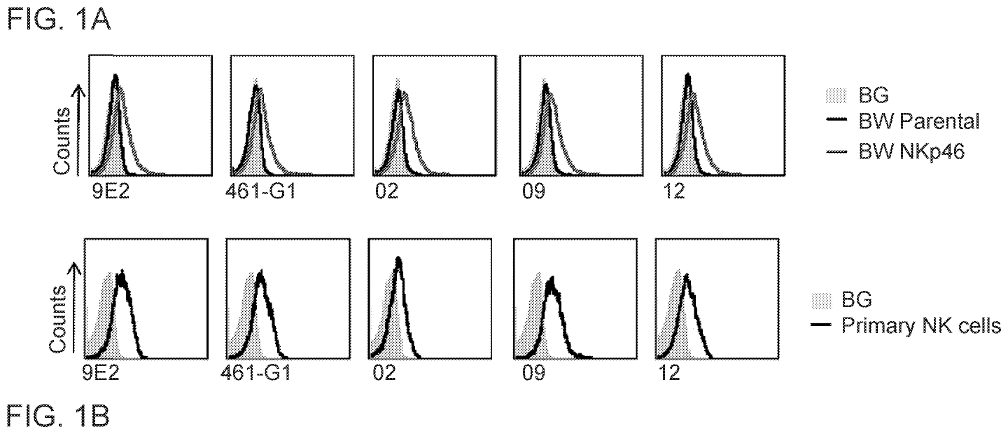

To develop antibodies against NKp46, NKp46-deficient mice (Ncr1^{gfp/gfp}, (Gazit et al., 2006)) were injected with a fusion protein consisting of the extracellular portion of NKp46 fused to human IgG (NKp46-Ig). Newly generated anti-NKp46 mAbs were evaluated the ability to bind NKp46. Data for clones 02, 09, and 12 are shown. Binding was examined on mouse thymoma BW transfectant cells expressing NKp46 (BW NKp46). A commercially available anti-NKp46 mAb (denoted 9E2) and an anti-NKp46 mAb (461-G1) (Amon et al., 2004; Mandelboim et al., 2001) were used as controls. Based on a flow cytometry experiment, all antibodies tested specifically interacted with BW NKp46 but not with the parental BW cells (FIG. 1A). This experiment was repeated with IL-2 activated primary bulk human NK cells (activated NK cells) to demonstrate that the antibodies recognizes NKp46 naturally expressed by human NK cells (FIG. 1B). The activated NK cells used throughout the experiments were isolated from PBMCs. Following purification, the NK cells were approximately 97% pure and were verified for CD56⁺CD3⁻ surface markers. All the antibodies (9E2, 461-G1, 02, 09 and 12) positively stained the activated NK cells. Similar results were obtained with PBMCs derived from additional donors.

Inhibition of Endogenous NKp46 Ligand Binding Using Mouse Anti-NKp46 mAbs

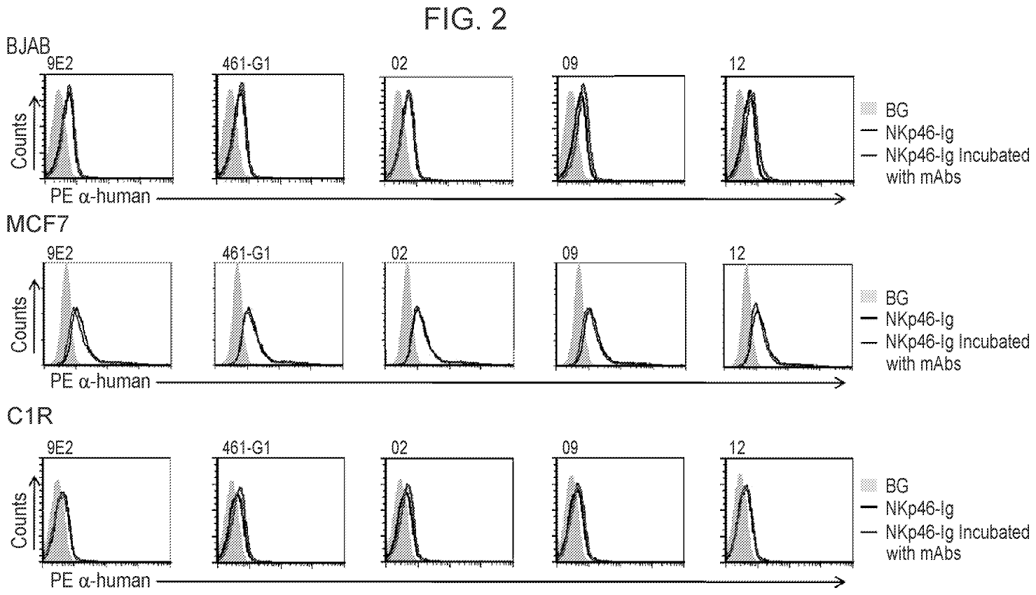

To determine if anti-NKp46 mAbs blocks the interaction of NKp46 with its ligands, BJAB, MCF7, and CIR tumor cells which express an unknown ligand for NKp46 were used (FIG. 2). NKp46-Ig was incubated either alone or with the anti-NKp46 mAbs and control antibodies on ice. Subsequently, the treated NKp46-Ig fusion proteins were used to FACS stain the tumor cells. None of the anti-NKp46 mAbs were able to block the binding of NKp46-Ig to the cells (FIG. 2).

### Downregulation of Surface Expressed NKp46 on NK Cells Using Mouse Anti-NKp46 Antibodies

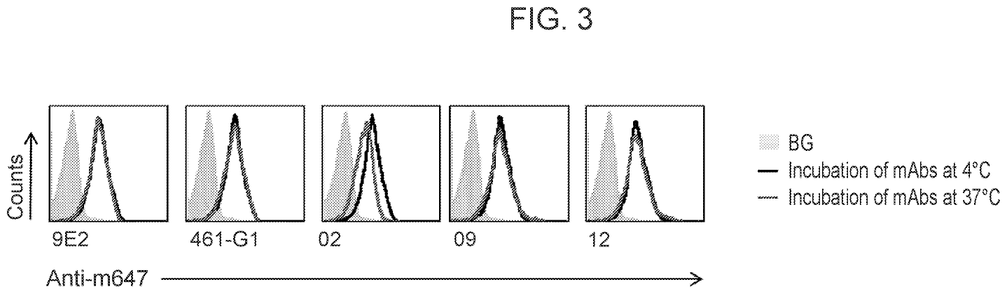

To test if anti-NKp46 mAbs leads to reduced NKp46 expression on the surface of NK cells human anti-NKp46 mAbs were incubated with activated NK cells for 8 hours either at 4° C. or 37° C. The cells were then FACS stained with a conjugated secondary anti-mouse antibody. Only one of the mAbs tested, 02, led to reduced levels of NKp46 (FIG. 3). Internalization assays were performed with only 9E2 control, 461-G1 control, and 02 and are described in (Berhani et al., 2018). Treatment of activated NK cells with 02 only led to an average surface downregulation of NKp46 by 60% (Berhani et al., 2018). 02 binding to NKp46, led to receptor internalization and degradation via the lysosomal pathway (Berhani et al., 2018). 12 and 09 anti-NKp46 antibodies did not lead to downregulation of NKp46 from the surface of NK cells.

Taken together, these assays revealed a new antibody which is unique in its ability to downregulate NKp46 surface expression, 02. Two other antibodies, 09 and 12, can be used to generate bispecific or tri-specific antibodies. These antibodies specifically bind NKp46, and they do not interfere with the binding of NKp46 to its cognate ligand.

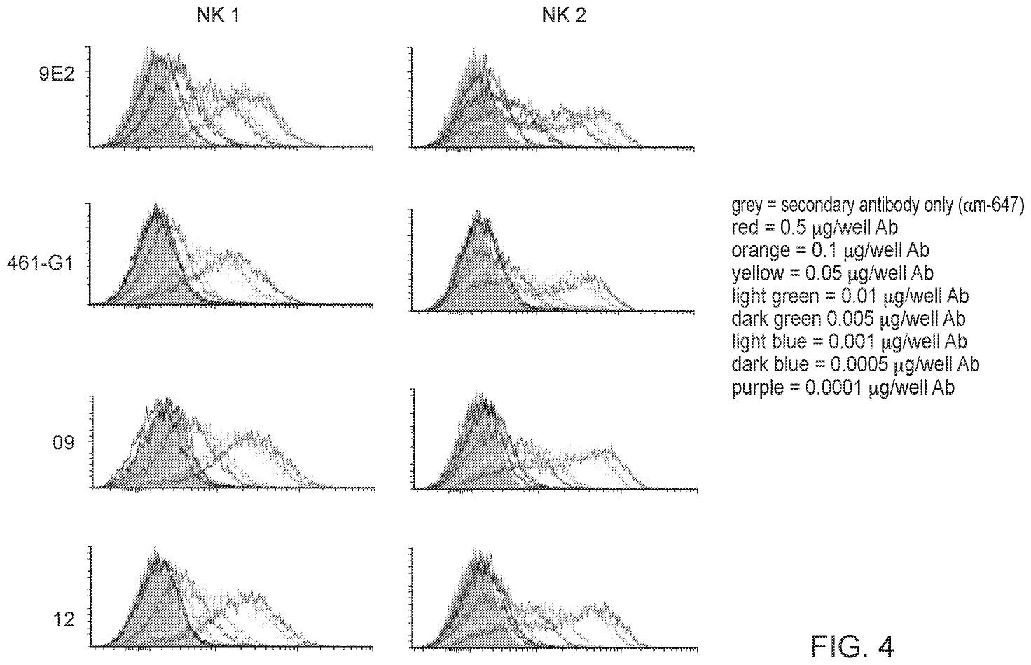

A dose response FACS staining with these antibodies on two primary activated primary NK cells was used to confirm the findings (FIG. 4). As the concentration of the tested antibodies decreases, the fluorescence signal in the FACs staining also decreases. Therefore, the 09 and 12 antibodies display a strong dose response. This is similar to the results seen using commercial anti-NKp46 control antibody and 461-G1 control antibody.

These results demonstrate that 09 and 12 ant-NKp46 antibodies are suitable for the generation of bispecific and tri-specific antibodies, which would bridge between NK cells and tumor cells; thus leading to tumor cell killing.

### BIAcore Binding Affinity

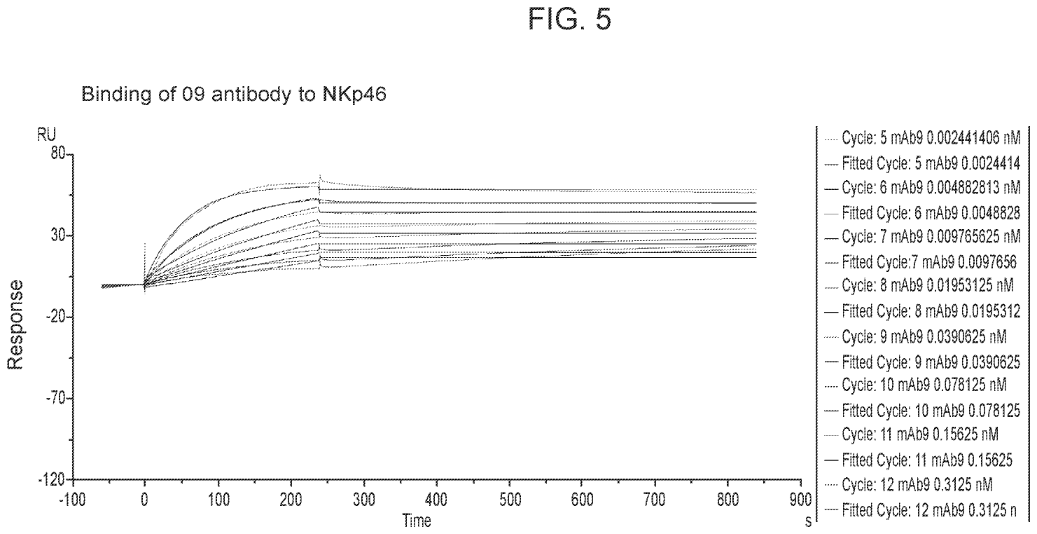

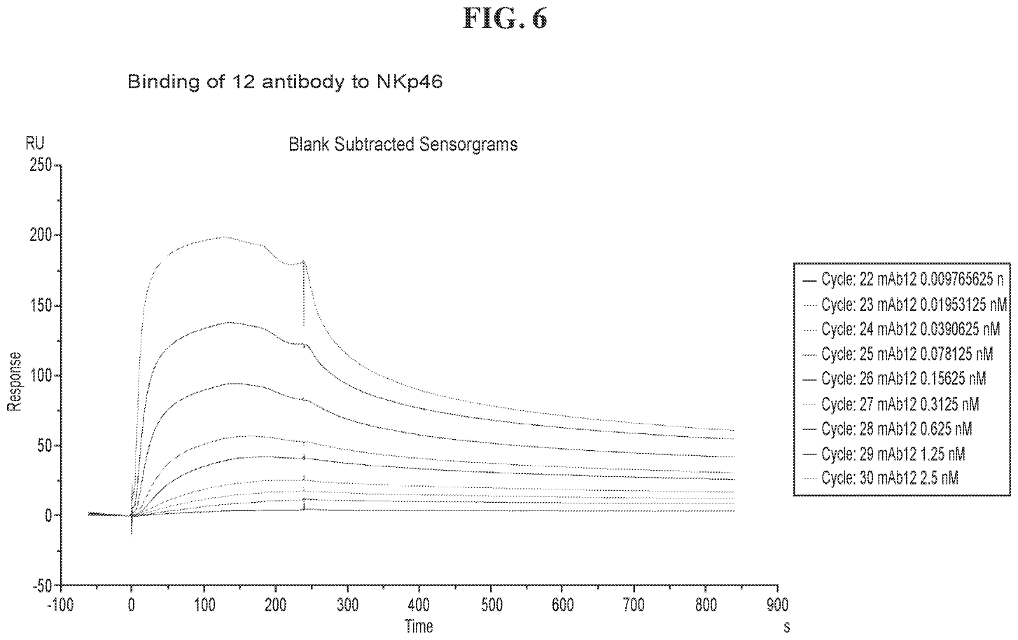

The binding affinity of two mouse anti-NKp46 (09 and 12) was determined using a BIAcore assay. FIG. 5 and Table 9 shows the binding affinity of 09 antibody. FIG. 6 and Table 10 shows the binding affinity of 12 antibody.

TABLE 9
Binding Affinity of 09 anti-NKp46 antibody determined by BIAcore

| ka | kd | KD | Rmax | Conc | | Flow | kt | RI | Chi² | U- |
|---|---|---|---|---|---|---|---|---|---|---|
| (1/Ms) | (1/s) | (M) | (RU) | (M) | tc | (ul/min) | (RU/Ms) | (RU) | (RU²) | value |
| 6.22E+07 | 1.50E−10 | 2.41E−18 | | | 1.69E+10 | | | | 4.68 | 95 |
| | | | 1009 | 2.44E−12 | | 30 | 5.24E+10 | 1.759- | | |
| | | | 408.1 | 4.88E−12 | | 30 | 5.24E+10 | −0.2504 | | |
| | | | 228.6 | 9.77E−12 | | 30 | 5.24E+10 | 0.4908 | | |
| | | | 141.4 | 1.95E−11 | | 30 | 5.24E+10 | 1.716 | | |
| | | | 89.95 | 3.91E−11 | | 30 | 5.24E+10 | 2.704 | | |
| | | | 66.19 | 7.81E−11 | | 30 | 5.24E+10 | 3.579 | | |
| | | | 56.02 | 1.56E−10 | | 30 | 5.24E+10 | 2.989 | | |
| | | | 59.06 | 3.13E−10 | | 30 | 5.24E+10 | 1.791 | | |

TABLE 10
Binding Affinity of 12 anti-NKp46
antibody determined by BIAcore

| | | Rmax | | offset | |
|---|---|---|---|---|---|
| KD (M) | SE(KD) | (RU) | SE(Rmax) | (RU) | SE(offset) |
| 1.67E−09 | 2.20E−10 | | | | |
| | | 317.8 | 19 | 6.1 | 2.1 |

### Example 2: Generation of Humanized Antibodies that Bind to Human NKp46

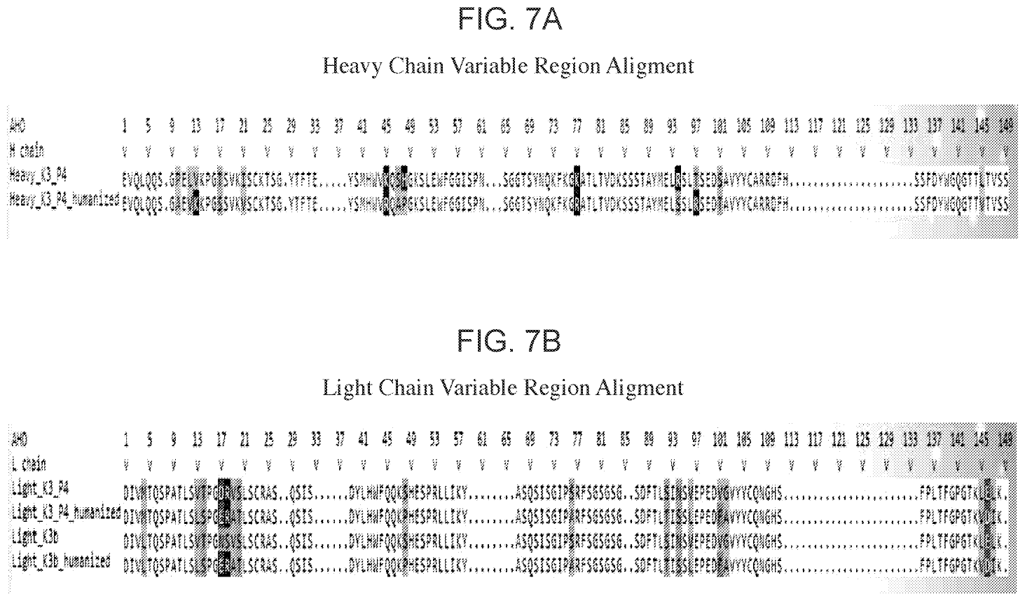

Mouse anti-NKp46 antibodies (09 antibody; 12 antibody) were used to generate humanized anti-NKp46 antibodies with an IgG4 framework 09 antibody humanized; 12 antibody humanized). FIG. 7A and FIG. 71B show the heavy chain variable region amino acid sequence alignment and the light chain variable region amino acid sequence alignment respectively. The resulting humanized sequences are set forth in SEQ ID NO: 25 (humanization of the heavy chain of 12) SEQ ID NO: 29 (humanization of the heavy chain of 09), SEQ ID NO: 26 (humanization of the Light chain of 09), and SEQ ID NO: 30 (humanization of the light chain of 12). Four humanized clones were prepared: B341001, B341002, B341003, and B341004.

(humanization of the heavy chain of 09),
>SEQ ID NO: 25
EVQLQQSGAEVKKPGSSVKVSCKTSGYTFTEYSMHWVRQAPGKSLEWFGG
ISPNSGGTSYNQKFKGRATLTVDKSSSTAYMELSSLRSEDTAVYYCARRD
FHSSFDYWGQGTTVTVSS
(humanization of the Light chain of 09)
>SEQ ID NO: 26
DIVMTQSPATLSLSPGERATLSCRASQSISDYLHWFQQKPHESPRLLIKY
ASQSISGIPARFSGSGSGSDFTLTISSLEPEDFAVYYCQNGHSFPLTFGP
GTKVDIK
(humanization of the heavy chain of 12):
SEQ ID NO: 29
EVQLQQSGAEVKKPGSSVKVSCKTSGYTFTEYSMHWVRQAPGKSLEWFGG
ISPNSGGTSYNQKFKGRATLTVDKSSSTAYMELSSLRSEDTAVYYCARRD
FHSSFDYWGQGTTVTVSS
(humanization of the light chain of 12)
>SEQ ID NO: 30
DIVLTQSPATLSLSPGERATLSCRASQSISDYLHWFQQKPHESPRLLIKY
ASQSISGIPARFSGSGSGSDFTLTISSLEPEDFAVYYCQNGHSFPLTFGP
GTKVDIKRTVAAPSVFIF

Binding of the humanized antibodies to NKp46 was tested using Human NKp46 antigen (Acro Biosystems) expressed on HEK293 cells or CHO cells.

1. Antigen coat tested in a matrix of 3 conc. vs. 3 buffers to find optimal signal.

2. Optimal signal conditions used to coat the ELISA tray. Standard blocking and washing steps.

3. Primary antibody was applied in range of concentrations with 10 dilutions. (MAbs with MMT humanized variable and mutated IgG4)

4. Secondary Anti Used to Detect Binding (AntiAnti-Hu Kappa with HRP, TMB Substrate)

5. Absorbance measured at 450 nm

Binding Data for Humanized 09 anti-NKp46 antibody is shown in Table 11. Using a normalized binding curve, the estimated K_{D} value is 27 μM.

TABLE 11
Humanized 09 anti-NKp46 Antibody Binding Data

| [09 antibody] nM | % Absorbance, Normalized |
|---|---|
| 5.218 | 0.709891 |
| 1.650 | 0.689258 |
| 0.5218 | 0.669464 |
| 0.1650 | 0.54814 |
| 0.05218 | 0.469117 |
| 0.01650 | 0.349274 |

Binding Data for Humanized 12 anti-NKp46 antibody is shown in Table 12. Using a normalized binding curve, the estimated K_{D} value is 25 μM.

TABLE 12
Humanized 12 anti-NKp46 Antibody Binding Data

| [12 antibody] nM | % Absorbance, Normalized |
|---|---|
| 5.218 | 0.720931 |
| 1.650 | 0.68579 |
| 0.5218 | 0.64718 |
| 0.1650 | 0.57056 |
| 0.05218 | 0.438264 |
| 0.01650 | 0.299833 |

### BIACore Assay

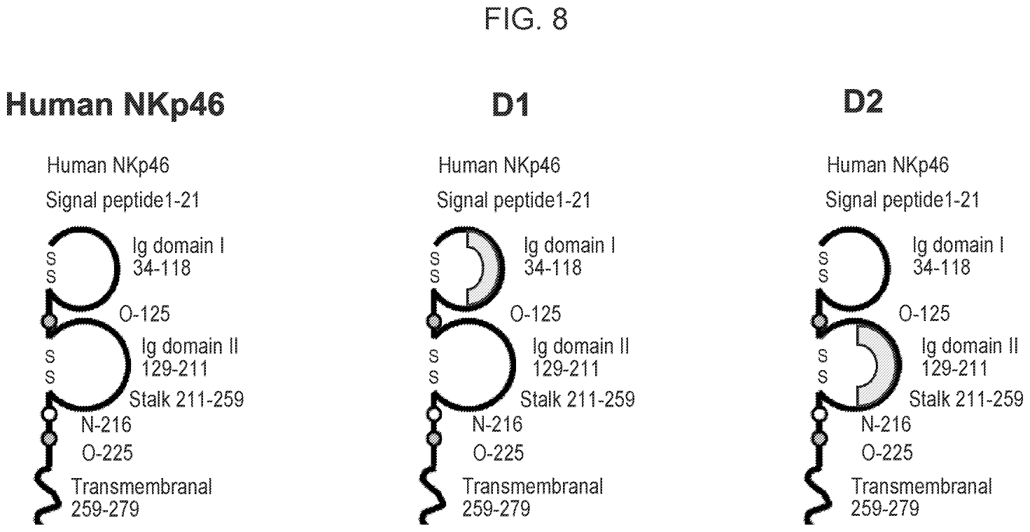

The binding affinity of the humanized anti-NKp46 antibodies (B341001, B341002, B341003, and B341004) to NKp46 domains was determined using a BIAcore assay. FIG. 8 shows the full length NKp46 polypeptide, D1 domain and D2 domain that were tested in these studies.

In this assay we incubated BW cells and BW cells transfected to express NKp46 (BW NKp46) with the mouse NKp46 antibodies 9 and 12, with the commercial mouse NKp46 antibody 9E9 and with the humanized NKp46 antibodies (B241001, B341002, B341003 and B341004). Antibodies stained the transfected BW NKp46 cells but not the parental BW cells indicating that the humanized NKp46 antibodies and the mouse antibodies are specific. As such, the antibodies bind specifically to the expressed NKp46.

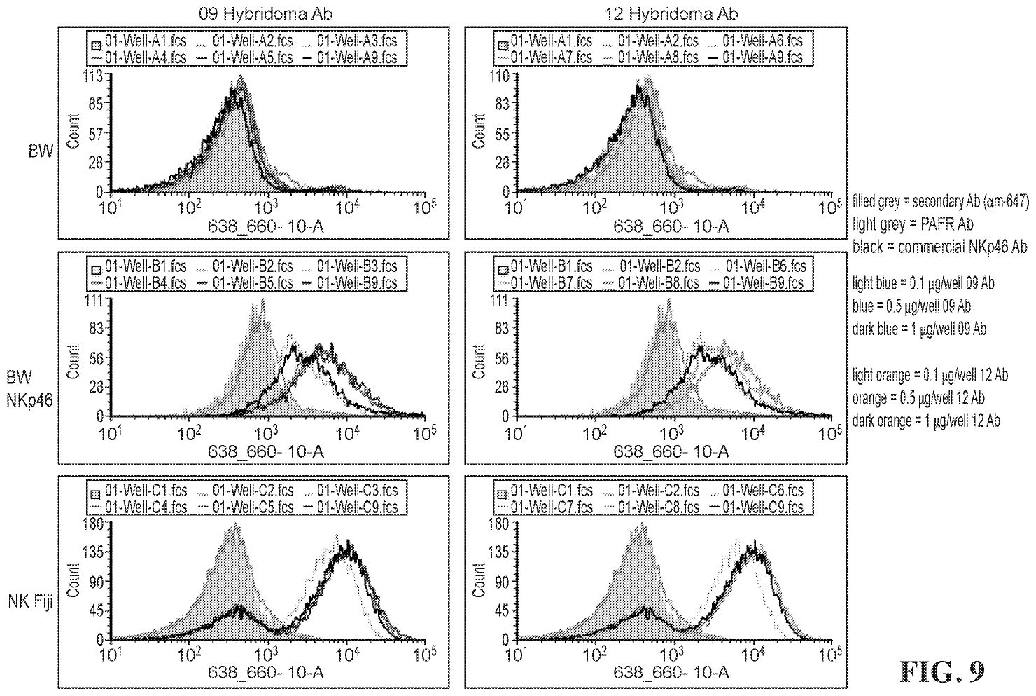

50,000 parental BW cells, BW cells transfected to express NKp46 or primary IL-2 activated NK cells (NK Fiji), were stained for NKp46 expression using mouse anti-human antibodies 9 and 12 at concentrations: 1 μg/ml, 5 μg/ml and 10 μg/ml. Anti-PAFR antibody was used as a negative control. FIG. 9 shows the results of these experiments.

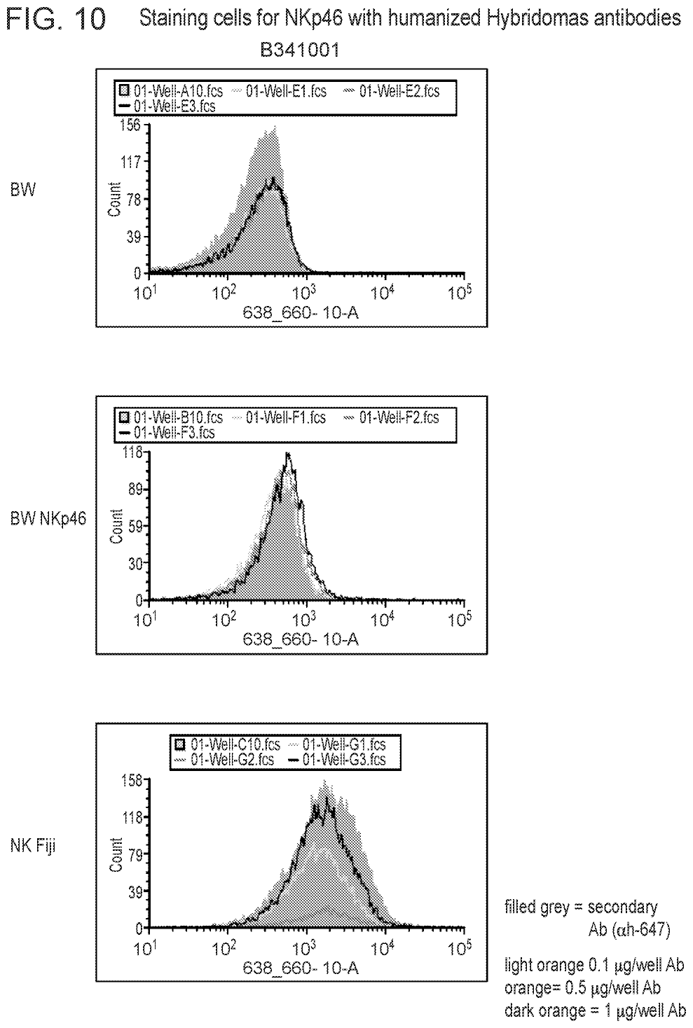

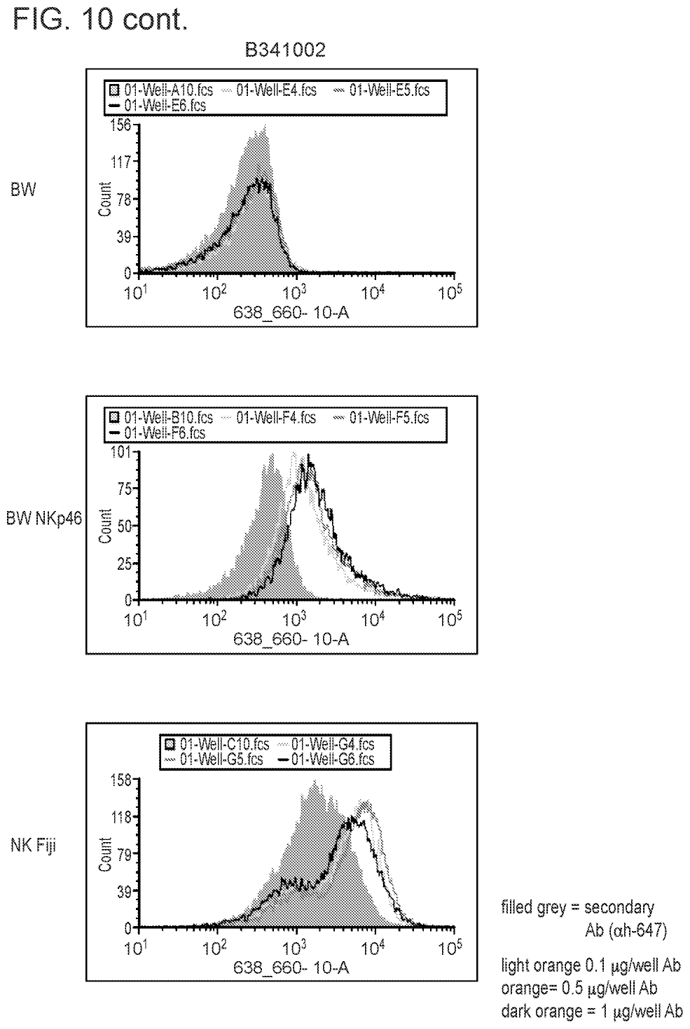

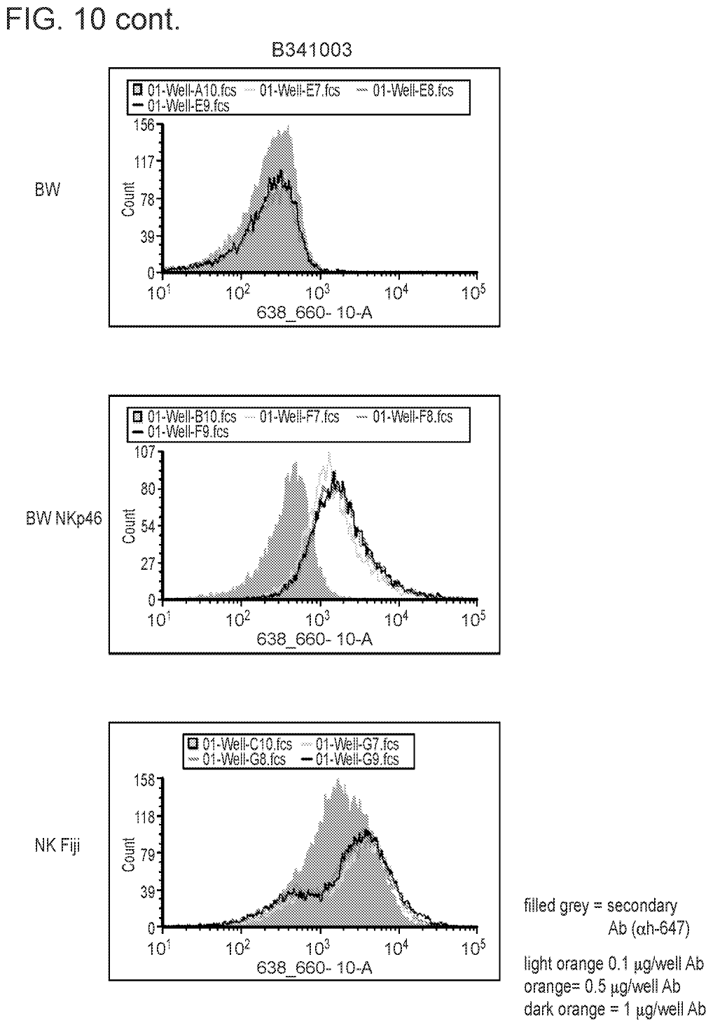

50,000 parental BW cells, BW cells transfected to express NKp46 or primary IL-2 activated NK cells (NK Fiji), were stained for NKp46 expression using various humanized anti-NKp46 antibodies at concentrations: 1 μg/ml, 5 μg/ml and 10 μg/ml. Anti-PAFR antibody was used as a negative control. FIG. 10 shows the results of these experiments.

As shown in Table 13, the humanized antibodies bound to D2 domain of the NKp46 polypeptide. 09 and 12 mouse anti-NKp46 monoclonal antibodies were included as a control and also bound to the D2 domain. The 09 anti-NKp46 monoclonal antibody shows binding to D1 domain also.

TABLE 13
Binding Affinity of humanized anti-
NKp46 antibodies to NKp46 domains

| | D1-Ig | D2-Ig | NKp46-Ig |
|---|---|---|---|
| B341001 | — | — | — |
| B341002 | — | 3.32E−16 | 2.53E−16 |
| B341003 | — | 2.48E−16 | 2.30E−17 |
| B341004 | — | 5.13E−16 | 1.76E−17 |
| Hyb 09 | 1.53E−14 | 3.68E−16 | 2.59E−16 |
| Hyb 12 | — | 3.09E−16 | 3.15E−16 |

### Example 3: Functional Characterization of Humanized Antibodies and Mouse Monoclonal Antibodies that Bind to Human Nkp46

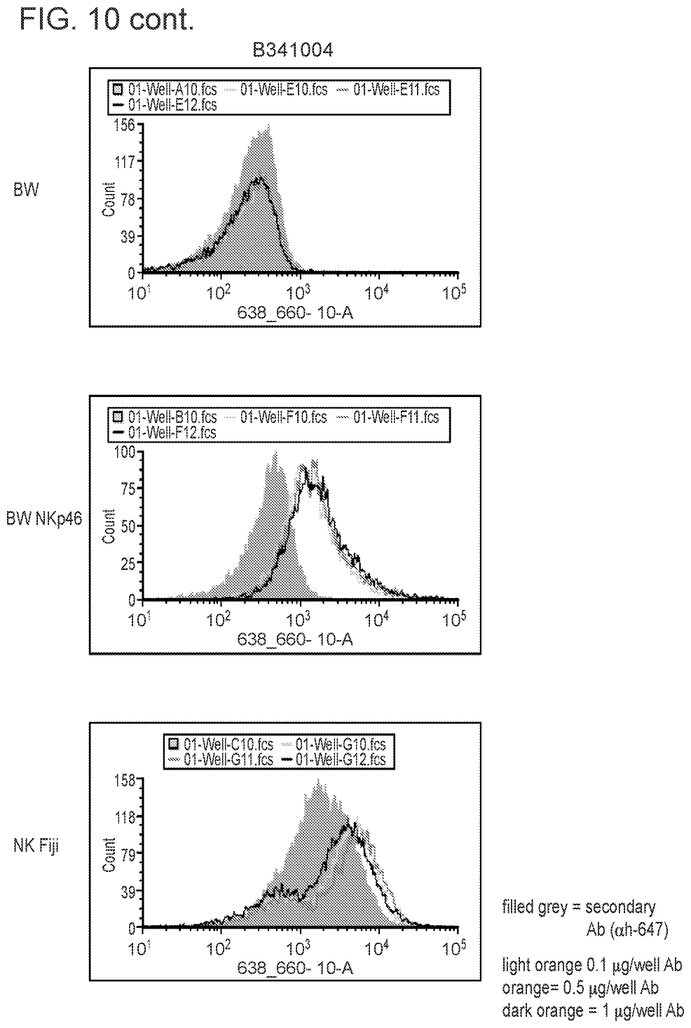

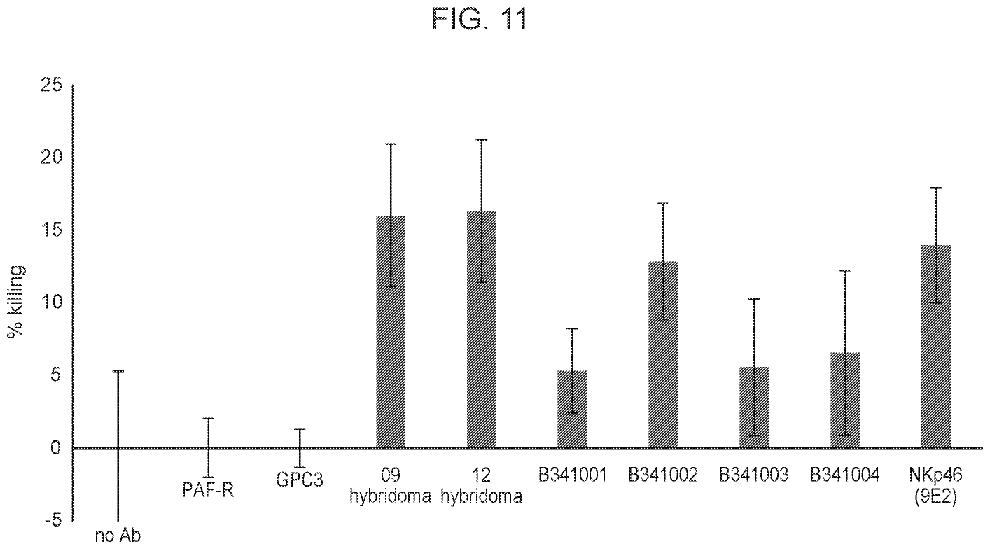

The humanized antibodies were examined for their ability to activate NK cells killing. 2500 mouse mastocytoma cell line P815 were incubated with 0.05 μg antibody for one hour on ice, then 10,000 NK cells were added and the cells were incubated for 5 hours at 37° C. PAR-R was use as a control antibody. An anti-GPC3 antibody was used as a control. No NK cell killing was observed using both control antibodies. 9E2 is a commercial anti-NKp46 antibody. FIG. 11 shows that the humanized antibodies activated NK cells killing.

Next, the humanized antibodies were examined for their ability to affect Killing of HepG2 cells (cells expressing GPC3) by NK cells. 5000 HepG2 cells were incubated with 1 μg or 5 μg of the mouse NKp46 antibodies 09 and 12, with the commercial mouse NKp46 antibody 9E9 and with the humanized NKp46 antibodies B241001, B341002, B341003 and B341004 for 1 hour on ice. Then, 100,000 NK cells were added and the cells were incubated for 5 hours at 37° C. FIG. 12 shows that none of the anti-NKp46 antibodies affect the killing of HepG2 cells.

### Example 4—Generation of Humanized CD38 Antibodies the Bind to Human CD38

Two humanized antibodies that bind to CD38 were generated (CD38-IgG1 L234A, L235A and CD38-IgG1 wildtype). Binding of the humanized antibody to human CD38 was tested. The ELISA plate was coated with 0.1 mg/well of human CD38 protein. 1t antibody (humanized anti-aNKp46 antibody or humanized anti-CD38 antibody) was added at 0.1 mg/well. Secondary antibody was diluted 1:7500. After addition of Streptavidin-HRP and TMB substrate the optical absorbance was measured at 650 nm. Anti-CD38 antibody specifically binds human CD38, while humanized anti-NKp46 control antibodies do not.

### Example 5: Generation of Bispecific Antibody Molecules that Bind to Human NKp46 and Human CD38

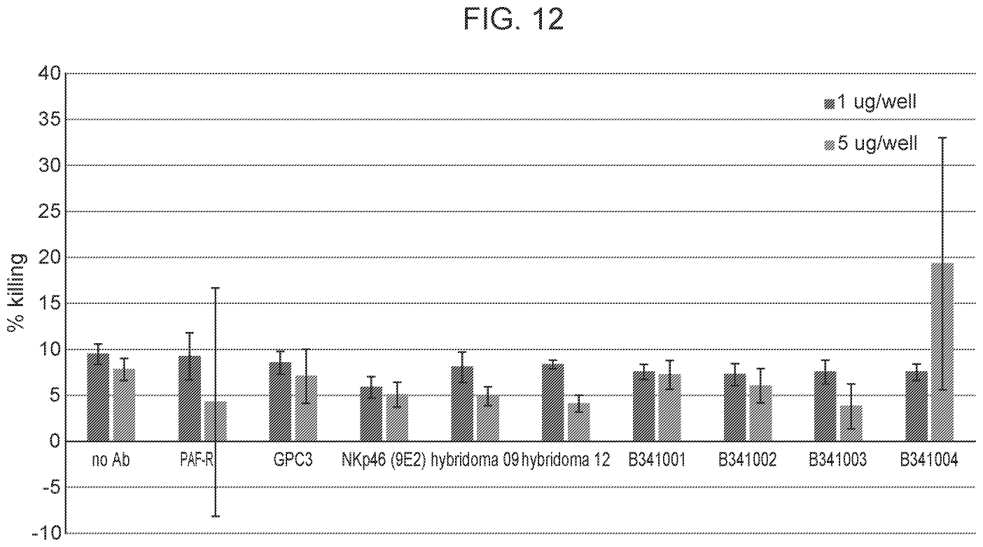

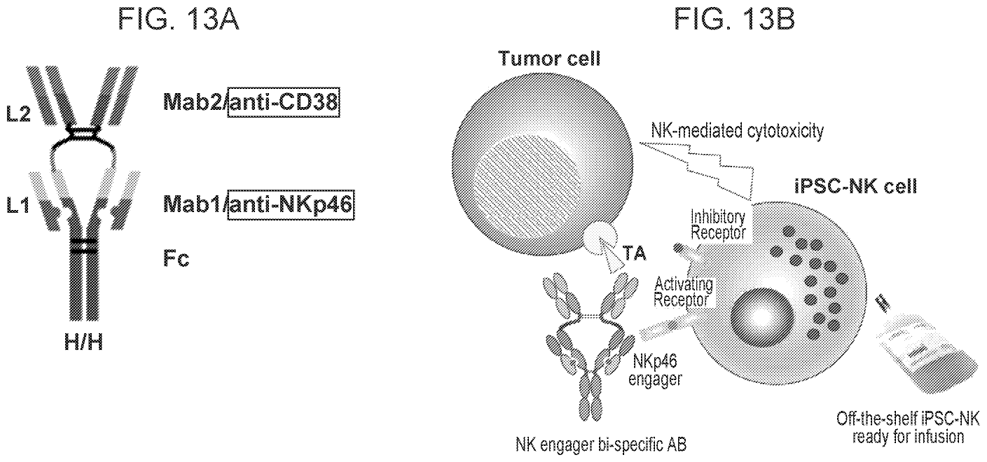

A bispecific antibody molecule that bind to human NKp46 and CD38 was constructed (NKE2). FIG. 13A shows a schematic diagram of the bispecific antibody molecules where mAb 1 has an anti-NKp46 antigen recognition region and mAb 2 an anti-CD38 antigen recognition region. In some cases, these bispecific antibody molecules are NK cell engager bispecific antibody molecules (FIG. 13B). The NK cell engager bispecific antibody can bind both an NK cell and a tumor cell and mediate NK-mediated cytotoxicity.

### Binding of Bispecific Antibody to Cells Expressing NKp46 and CD38

Three different types of cells were used to test binding of bispecific antibody molecules to NKp46 and CD38—BW cells that express neither NKp46 nor CD38, BW cells expressing NKp46, and KMS11 and MM1.S MM cell lines expressing CD38. 50,000 cells were incubated with 0.5 μg of anti-CD38 and NKp46 bispecific antibody, anti-CD38 antibody, or no antibody (control) for 1 hour on ice, then AF-647 conjugated anti-human antibody was added for another 30 minutes. This experiment shows that the bi-specific antibody binds to NKp46 (using the BW NKp46 arm) and to KMS11 or MM1.S MM cells using the anti-CD38 arm.

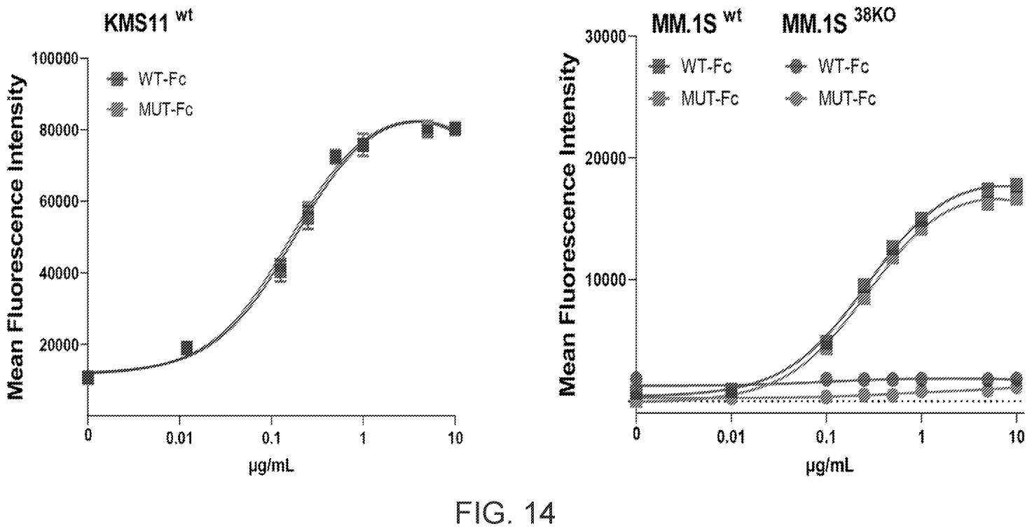

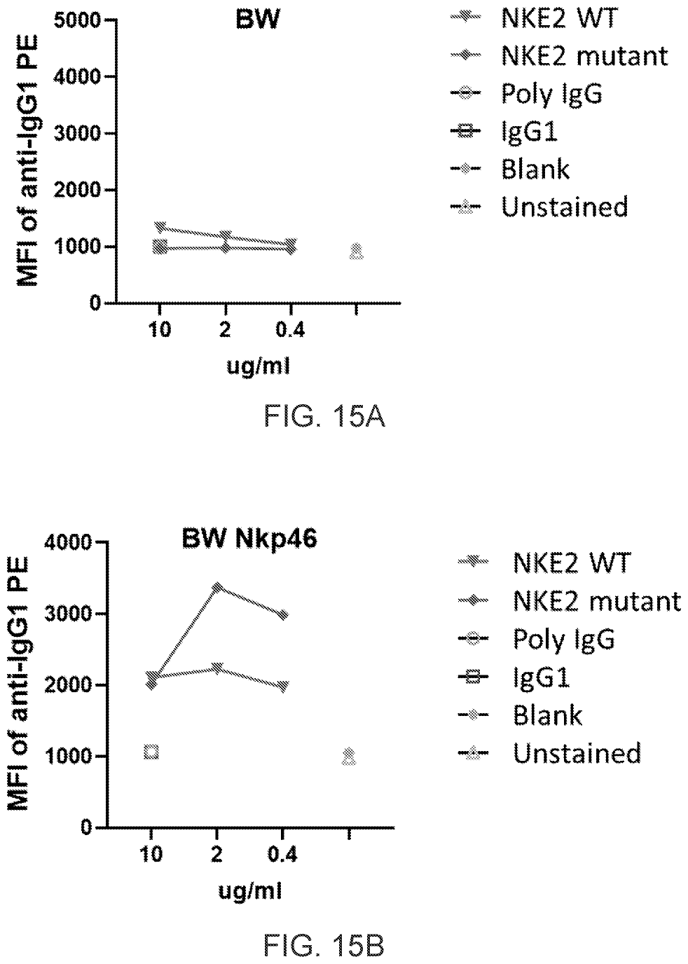

Dose dependent binding was observed with NKE2 WT or Mut Fc (Fc silent) to MM1S and KMS11 multiple myeloma cells, and no binding to a CD38 knock out MM.1S cell line was observed (FIG. 14). Similar binding was observed with NKE2 WT and Mut Fc to BW cells (FIGS. 15A and 15B).

### Example 6: Functional Characterization of Bispecific Antibody Molecules that Bind to Human NKp46 and Human CD38

### KMS11 and MMS.1 Cell Killing by NK Cells—Radioactive Assay

KMS11 and MMS.1 cells express CD38. To determine if KMS11 and MMS.1 MM cells can be killed by NK cells in the presence of a bispecific antibody that binds to NKp46 and CD38, KMS11 and MMS.1 MM cells were radioactively labeled with ³⁵S-Methionine and plated in a 96 plate at 5000 cells/well. Primary activated human NK cells were added to the wells at different amounts for different effector to target (E:T) ratios (100,000, 50,000, 25,000 and 12,500 cells per well, 20:1, 10:1, 5:1 and 2.5:1 ratios). The cells were incubated for 5 hrs at 37° C., and the medium was harvested and radioactivity was determined using a beta counter. KMS11 and MMS.1 MM cell was measured in the presence of the bispecific antibody at various E:T ratios.

### KMS11 and MMS.1 MM Cell Killing by NK Cells—Degranulation Assay

Different amounts of KMS11 and MMS.1 MM cells were plated in a 96 plate (500,000, 250,000, 125,000, 62,500 31,250, 15,625 and 7,800 cells/well). 5000 primary activated human NK cells were then added together with aCD56 and aCD107A antibodies. Cells were incubated at 37° C. for 2 hrs. NK degranulation was calculated by Flow Cytometry staining of CD107 on the CD56 positive cells. Cells were incubated with an anti-NKp46 and anti-CD38 bispecific antibody an anti-NKp46 antibody control and an anti-CD38 antibody control. The percentage of degranulation exhibited was measured in the bispecific antibody at various E:T ratios. Degranulation is a proxy for KMS11 and MMS.1 MM cell killing by NK cells. Thus, this shows that bispecific antibody that binds both NKp46 and CD38 has an increased functional effect on KMS11 and MMS.1 MM cell killing than the monospecific antibodies alone.

### Example 7: In Vivo Functional Characterization of Bispecific Antibody Molecules that Bind to Human NKp46 and Human CD38

Effects of Bispecific Antibody that Binds Human NKp46 and CD38 in a Human MM Xenograft Model

MM1.S cells expressing luciferase (MM1.Sluc) are injected in the tail vein of immunodeficient (NSG) mice and luciferase levels are measured by IVIS Imaging System every 3 or 4 days. Thirteen days after MM1.S injection, purified human T cells are transplanted I.V. with or without anti-NKp46 and CD38 bispecific antibody. Dose response experiments to determine a safe amount of bi-specific antibodies needed to reduce tumor size are conducted. Increasing concentrations of the bi-specific antibodies and the singular antibodies (humanized anti-NKp46 and anti-CD38) are injected into mice bearing tumors. Antibody injections are administered twice a week. For a period of 4 weeks, mice are monitored daily (weight of mice, and general appearance) and their tumors measured by digital vernier caliper. The humane endpoint is set to a tumor volume of 1 cm³ or a weight loss of 20% from initial body weight. After four weeks, mice are sacrificed and tumors are be removed and weighed. Treatments in which a significant inhibition of tumor growth (lower tumor volume and weight) is observed would be considered as successful.

### Example 8: Characterization of a Novel Multifunctional Tetravalent CD38 NKp46 FLEX-NK™ Engager

CD38 is a clinically validated target for NK cell mediated cytotoxicity in multiple myeloma. NKE2 is a tetravalent IgG1-like multifunctional NK engager antibodies with a novel FLEX-linker which simultaneously binds both the targeted cancer cells and NK cells. NK engagement and activation is mediated through a binder directed against the natural cytotoxicity receptor NKp46. NKE2 comprises an anti-CD38 binding domain and the NKp47 binding domain of the humanized 09 antibody (see tables 1-6 for sequences). In this example, two versions of NKE2, one with a wild type (WT) Fc and one with a mutant null Fc were characterized NKE2 showed dose dependent binding to multiple myeloma cell lines

Binding of NKE2 to CD38 expressing multiple myeloma cell lines MM1S and KMS11 and to a CD38 knock out MM.1S cell line was determined. Dose dependent binding was observed with NKE2 WT or Mut Fc (Fc silent) to MM1S and KMS11 multiple myeloma cells, and no binding to a CD38 knock out MM.1S cell line was observed (FIG. 14).

### NKE2 Showed Dose Dependent Binding to NK Cells

NKE2 binding to BW cells was determined. BW parental cells or human Nkp46 transfected BW Cells were incubated with NKE2 or Control IgG1 for 1 hour at RT. Then the cells were stained with Secondary anti-IgG1 PE Ab and the binding (MFI of anti-IgG1 PE) was measured by flow cytometry. Results are shown in FIGS. 15A and 15B. Similar binding was observed with NKE2 WT and Mut Fc to BW cells.

Epitope Mapping Studies Indicate NKE2 Binding to a Distinct Site from Daratumumab

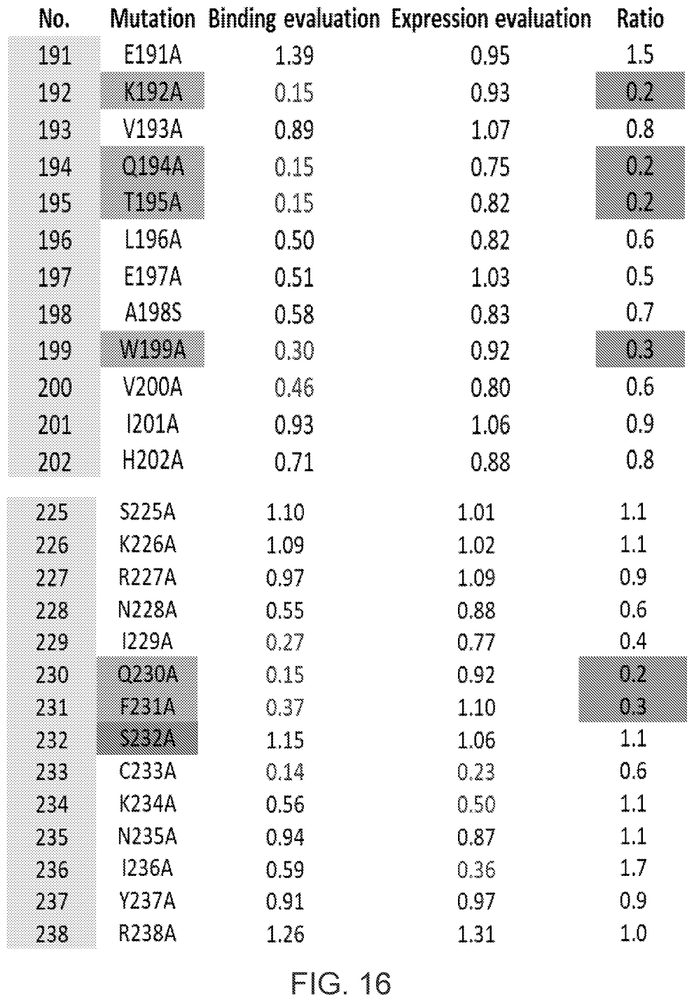

NKE2 epitope mapping analysis by CD38 alanine scanning mutagenesis indicates NKE2 binds a distinct site from Daratumumab. Results are shown in FIG. 16. Alanine mutants that affect NKE2 binding are highlighted in green and the residue that's known to affect Daratumumab binding is highlighted in red (residue S232 corresponds to S274 with the leader sequence). These data suggest a distinct epitope detected by NKE2.

### NKE2 Showed Dose Dependent PBNK Redirected Cytolysis of Multiple Myeloma Cell Lines.

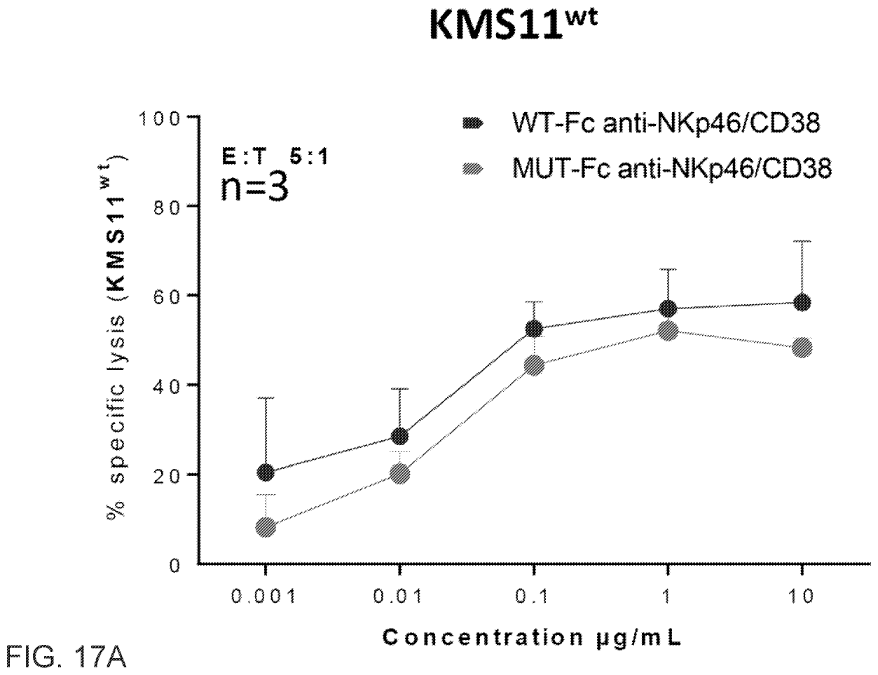

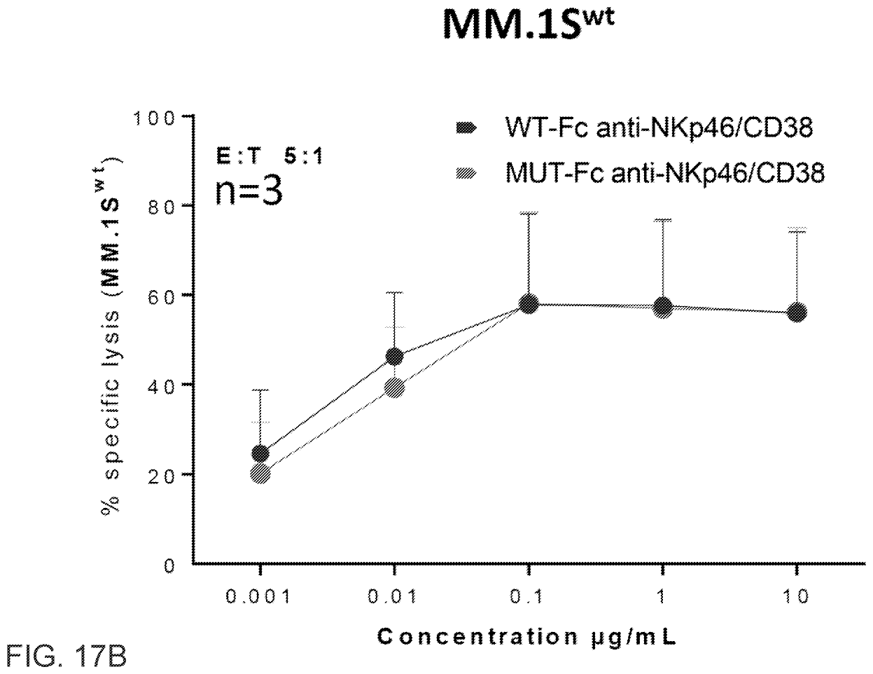

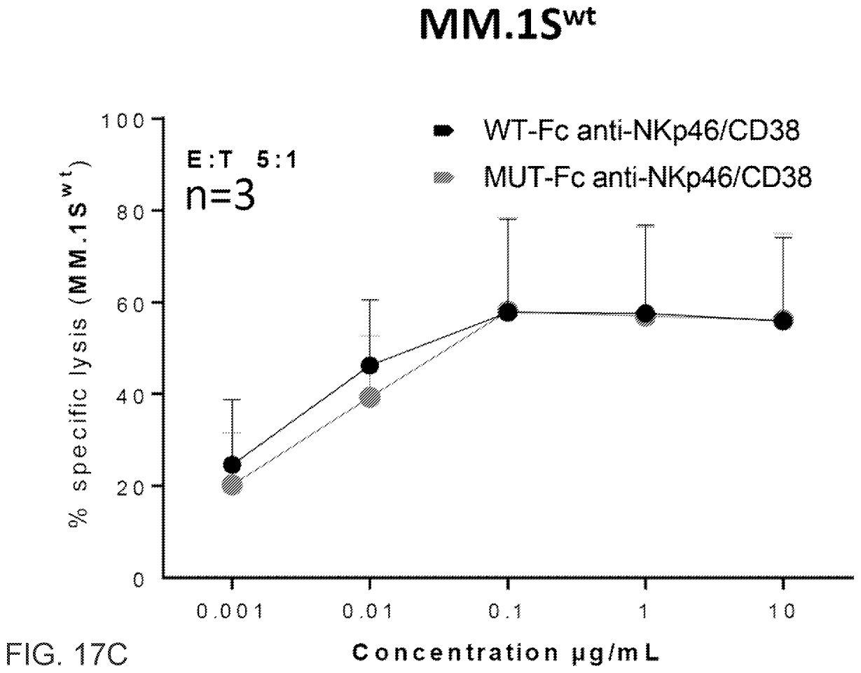

PBNK cytotoxicity assay was assessed with increasing NKE2 concentrations at a fixed E/T ratio of 5 for 20 hrs and target cytolysis assessed by flowcytometry. Results are shown in FIGS. 17A-17C. NKE2 dose-dependent PBNK redirected cytolysis of KMS11 and MM1S multiple myeloma cells was dependent on CD38 expression as minimal cytolysis was observed with MM1S^{CD38KO} cells. NKE2 WT-Fc showed slightly higher cytolysis compared NKE2 Fc mutant.

### NKE2 PB-NK Cell Redirected Cytolysis, Degranulation and Cytokine Production Against Multiple Myeloma MMTS Cells

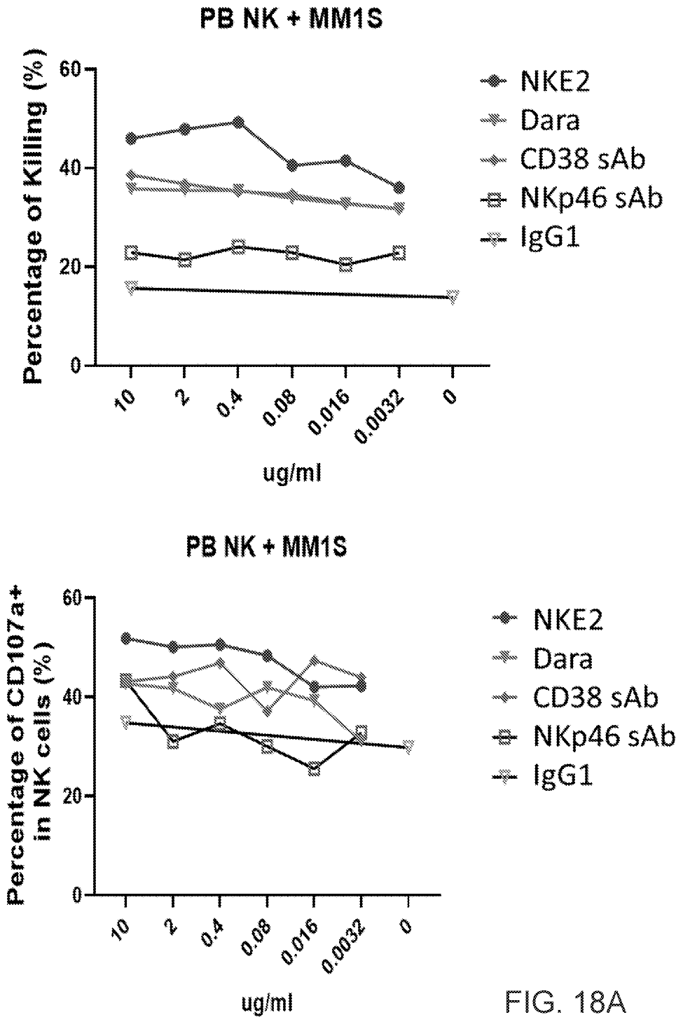

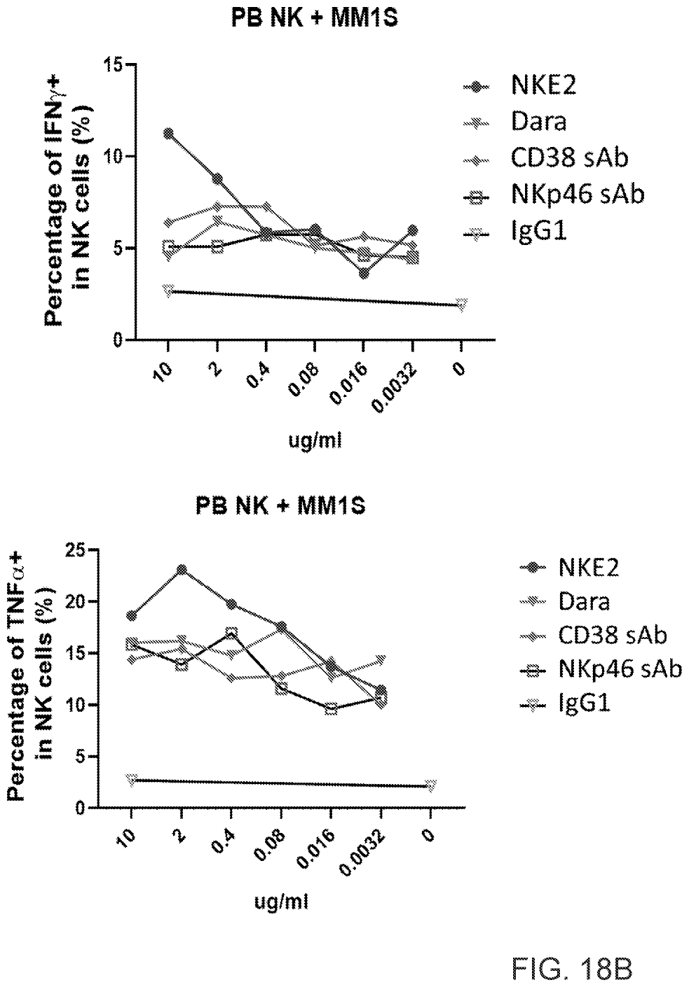

PB-NK cytolysis was determined at various concentrations of NKE2 at a fixed E/T ratio of 1 for 24 hrs and target lysis analyzed by flow cytometry. PBNK degranulation was assessed at an E/T ratio of 1 for 5 hrs and assessed by flowcytometry using an anti-CD107a antibody. Results are shown in FIG. 18A. NKE2 showed significantly higher dose dependent PB-NK cell redirected cytolysis and degranulation against multiple myeloma MM1S cells compared to Daratumumab or single CD38 and NKp46 mAb's. These results indicate that co-engagement of NKp46 and CD38 on opposing NK and MM cells by NKE2 contributes to the higher potency observed by this NK cell engager. Interestingly, the NKp46 mAb alone without a functional Fc induced peripheral blood NK cell cytolysis of the multiple myeloma cells, consistent with a prior report that NKp46 plays a key role in NK-cell mediated killing of myeloma cells.

Cytokine production was assessed by intracellular staining for IFN-g and TNF-α by flowcytometry. Results are shown in FIG. 18B. NKE2 also induced higher % of IFN-g and TNF-α producing PBNK cells compared to Daratumumab and single CD38 and NKp46 mAbs following incubation with MM cells at E/T ratio of 1 for 5 hrs.

### NKE2 Showed No Significant Immune Subset Depletion and Low Levels of NK Cell Fratricide and Cytokine Release in Human PBMC's In-Vitro

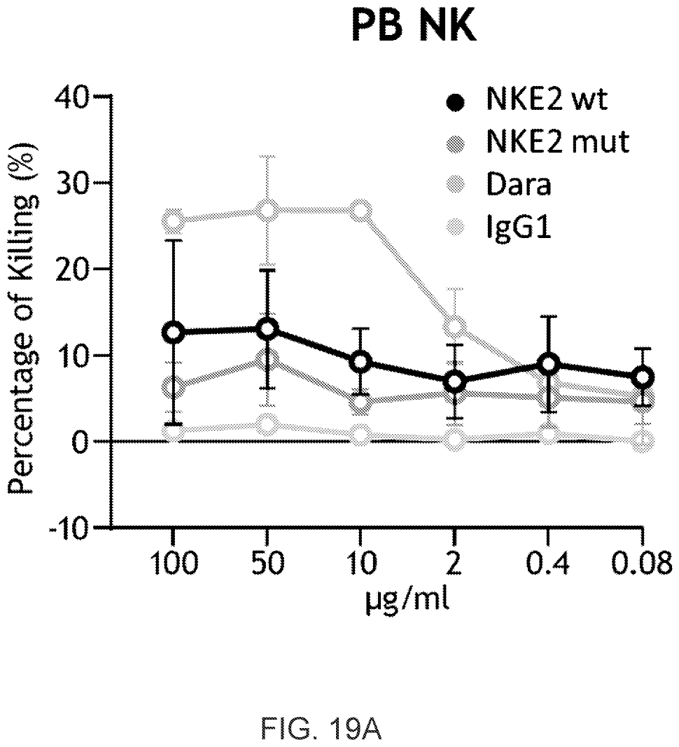

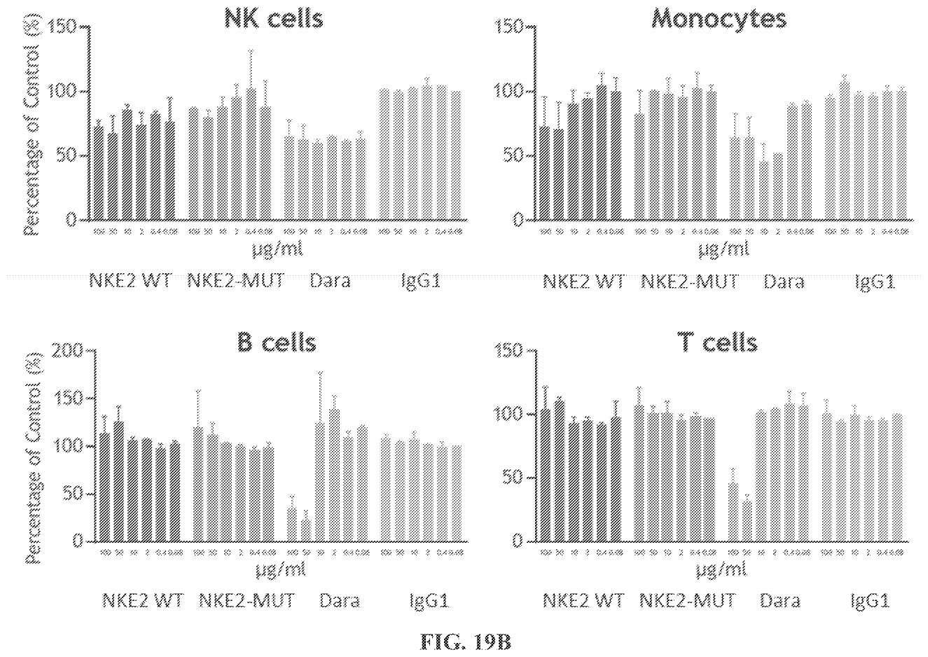

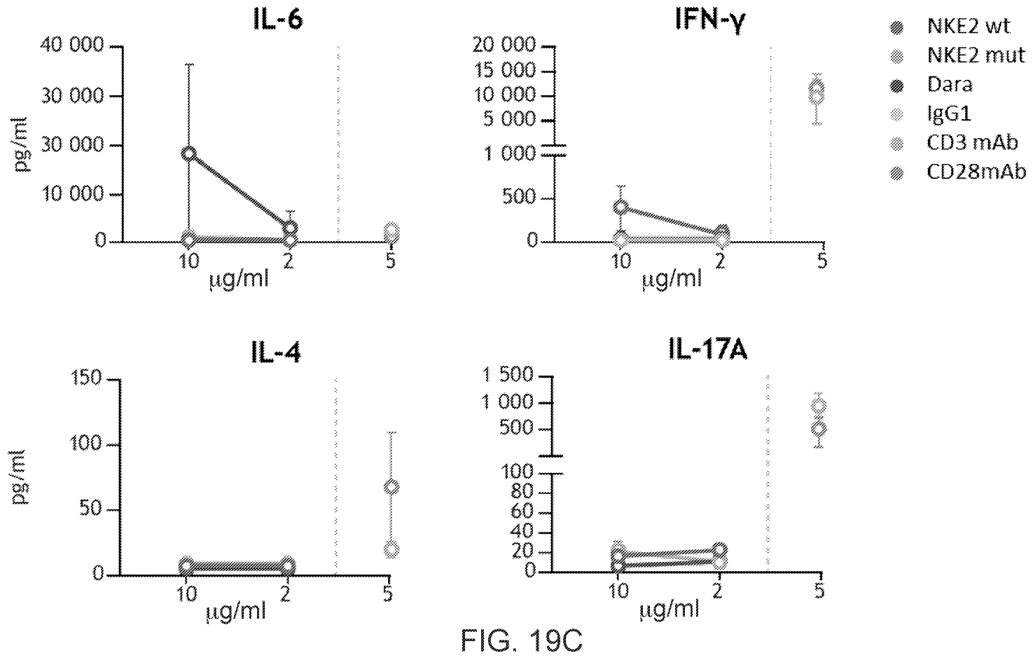

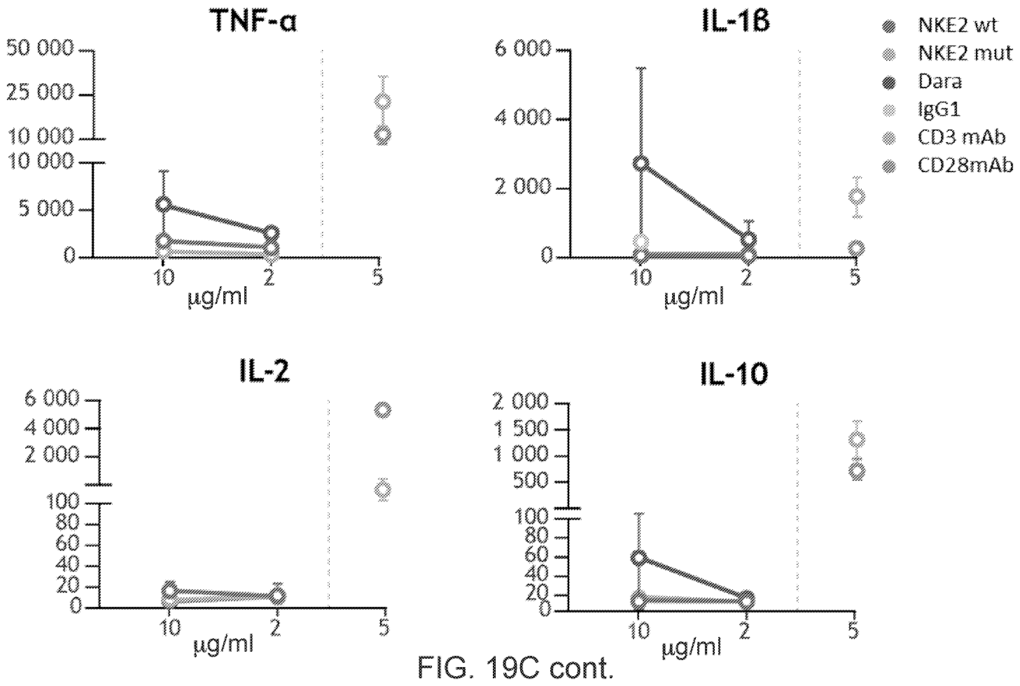

NK cell fratricide was evaluated using purified PBNK in the presence of NKE2 or daratumumab or human IgG by flow cytometry using the live dead cell dye. Results are shown in FIG. 19A. Daratumumab showed higher fratricide of PBNK cells compared to NKE2 WT and NKE2 MUT.

The effect of NKE2 on human PBMC immune cell subset depletion was evaluated following incubation with NKE2 or daratumumab or hIgG1 for 24 hrs followed by immune subset analysis by flow cytometry. Results are shown in FIG. 19B. While daratumumab showed depletion of NK cells and monocytes, minimal immune cell depletion was observed with NKE2 WT and NKE2 MUT.

The potential of NKE2 to induce cytokine release was evaluated in the human PBMC assay following incubation with NKE2 or anti-CD3 or CD28 mAbs (TGN1412) or hIgG1 for 48 hrs and supernatants tested for the presence of cytokines by multiplex ELISA assay. Results are shown in FIG. 19C. While robust cytokine release was observed with anti-CD3 and CD28 (TGN1412) mAbs, minimal cytokine release was observed with NKE2.

### CONCLUSION

The results discussed in this example suggest that the CD38 NKp46 engagers have a favorable NK cell engager profile for targeting CD38 expressing multiple myeloma distinct from Daratumumab.

### INCORPORATION BY REFERENCE

All publications, patents, and Accession numbers mentioned herein are hereby incorporated by reference in their entirety as if each individual publication or patent was specifically and individually indicated to be incorporated by reference.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A bispecific antibody that specifically binds NKp46 and CD38, comprising:
i) a first heavy chain comprising
a heavy chain complementarity determining region 1 (CDRH1) comprising an amino acid sequence of SEQ ID NO: 17;
a heavy chain complementarity determining region 2 (CDRH2) comprising an amino acid sequence of SEQ ID NO: 18; and
a heavy chain complementarity determining region 3 (CDRH3) comprising an amino acid sequence of SEQ ID NO: 19;
ii) a first light chain comprising
a light chain complementarity determining region 1 (CDRL1) comprising an amino acid sequence of SEQ ID NO: 20;
a light chain complementarity determining region 2 (CDRL2) comprising an amino acid sequence of SEQ ID NO: 21; and
a light chain complementarity determining region 3 (CDRL3) comprising an amino acid sequence of SEQ ID NO: 22;
iii) a second heavy chain comprising
a CDRH1 comprising an amino acid sequence of SEQ ID NO: 32;
a CDRH2 comprising an amino acid sequence of SEQ ID NO: 33; and
a CDRH3 comprising an amino acid sequence of SEQ ID NO: 34; and
iv) a second light chain comprising
a CDRL1 comprising an amino acid sequence of SEQ ID NO: 35;
a CDRL2 comprising an amino acid sequence of SEQ ID NO: 36; and
a CDRL3 comprising an amino acid sequence of SEQ ID NO: 37; and
wherein the bispecific antibody comprises a first antigen binding region comprising i) and ii) that specifically binds to NKp46 and a second antigen binding region comprising iii) and iv) that specifically binds to CD38.

2. The bispecific antibody of claim 1, wherein
the first heavy chain comprises a first heavy chain variable region comprising an amino acid sequence of SEQ ID NO: 23, 25, 27 or 29;
the first light chain comprises a first light chain variable region comprising an amino acid sequence of SEQ ID NO: 24, 26, 28 or 30;
the second heavy chain comprises a second heavy chain variable region comprising an amino acid sequence of SEQ ID NO: 38; and
the second light chain comprises a second light chain variable region comprising an amino acid sequence of SEQ ID NO: 39.

3. The bispecific antibody of claim 1, wherein the first antigen binding region comprises
a) a first heavy chain comprising a first heavy chain variable region comprising an amino acid sequence of SEQ ID NO: 23 and a first light chain comprising a first light chain variable region comprising the amino acid sequence of SEQ ID NO: 24;
b) a first heavy chain comprising a first heavy chain variable region comprising an amino acid sequence of SEQ ID NO: 25 and a first light chain comprising a first light chain variable region comprising the amino acid sequence of SEQ ID NO: 26;
c) a first heavy chain comprising a first heavy chain variable region comprising an amino acid sequence of SEQ ID NO: 27 and a first light chain comprising a first light chain variable region comprising the amino acid sequence of SEQ ID NO: 28; or
d) a first heavy chain comprising a first heavy chain variable region comprising an amino acid sequence of SEQ ID NO: 29 and a first light chain comprising a first light chain variable region comprising the amino acid sequence of SEQ ID NO: 30.

4. The bispecific antibody of claim 1, wherein the second antigen binding region comprises:
a second heavy chain comprising a second heavy chain variable region comprising an amino acid sequence of SEQ ID NO: 38 and a second light chain comprising a second light chain variable region comprising the amino acid sequence of SEQ ID NO: 39.

5. The bispecific antibody of claim 1, wherein
a) the first heavy chain comprises a first heavy chain variable region comprising an amino acid sequence of SEQ ID NO: 23; the first light chain comprises a first light chain variable region comprising an amino acid sequence of SEQ ID NO: 24; the second heavy chain comprises a second heavy chain variable region comprising an amino acid sequence of SEQ ID NO: 38; and the second light chain comprises a second light chain variable region comprising an amino acid sequence of SEQ ID NO: 39;
b) the first heavy chain comprises a first heavy chain variable region comprising an amino acid sequence of SEQ ID NO: 25; the first light chain comprises a first light chain variable region comprising an amino acid sequence of SEQ ID NO: 26; the second heavy chain comprises a second heavy chain variable region comprising an amino acid sequence of SEQ ID NO: 38; and the second light chain comprises a second light chain variable region comprising an amino acid sequence of SEQ ID NO: 39;
c) the first heavy chain comprises a first heavy chain variable region comprising an amino acid sequence of SEQ ID NO: 27; the first light chain comprises a first light chain variable region comprising an amino acid sequence of SEQ ID NO: 28; the second heavy chain comprises a second heavy chain variable region comprising an amino acid sequence of SEQ ID NO: 38; and the second light chain comprises a second light chain variable region comprising an amino acid sequence of SEQ ID NO: 39; or
d) the first heavy chain comprises a first heavy chain variable region comprising an amino acid sequence of SEQ ID NO: 29; the first light chain comprises a first light chain variable region comprising an amino acid sequence of SEQ ID NO: 30; the second heavy chain comprises a second heavy chain variable region comprising an amino acid sequence of SEQ ID NO: 38; and the second light chain comprises a second light chain variable region comprising an amino acid sequence of SEQ ID NO: 39.

6. The bispecific antibody of claim 1, wherein the bispecific antibody comprises a fused heavy chain comprising an amino acid sequence of SEQ ID NO: 41, a first light chain comprising an amino acid sequence of SEQ ID NO: 31 and a second light chain comprising an amino acid sequence of SEQ ID NO: 40.

7. The bispecific antibody of claim 1, wherein the bispecific antibody comprises an amino acid sequence of SEQ ID NO: 46, a first light chain comprising an amino acid sequence of SEQ ID NO: 31 and a second light chain comprising an amino acid sequence of SEQ ID NO: 40.

8. The bispecific antibody of any one of claims 1-7, wherein the bispecific antibody comprises at least two Fab fragments with different CH1 and CL domains, wherein said Fab fragments comprise:
a) a first Fab fragment consisting of:
i. the VH region and VL region that specifically binds NKp46;
ii. a CH1 domain of a human immunoglobulin comprising substitution of the threonine residue at position 192 of said CH1 domain with a glutamic acid residue; and
iii. a CL-kappa domain of a human immunoglobulin comprising substitution of the asparagine residue at position 137 of said CL domain with a lysine residue and substitution of the serine residue at position 114 of said CL domain with an alanine residue,
b) a second Fab fragment consisting of wild-type human CH1 and wild-type human CL domains of an immunoglobulin, and the VH region and VL region that specifically binds CD38; and
wherein the sequence position numbers used for the CH1 and CL domains refer to Kabat numbering and Fab fragments are tandemly arranged in any order, and
wherein the C-terminal end of the CH1 domain of the first Fab fragment being linked to the N-terminal end of the VH domain of the following Fab fragment is through a polypeptide linker.

9. The bispecific antibody of claim 8, wherein the polypeptide linker comprises an amino acid sequence of SEQ ID NO: 9 or 44.

10. The bispecific antibody of claim 8, further comprising
c) a dimerized CH2 domain and CH3 domain of an immunoglobulin; and
d) a hinge region of an IgA, IgG, or IgD, linking the C-terminal ends of the CH1 domains of the antigen binding region to the N-terminal ends of the CH2 domains.

11. The bispecific antibody of claim 8, further comprising an Fc domain derived from an IgG1 Fc domain or an IgG4 Fc domain.

12. The bispecific antibody of claim 11, wherein the Fc domain region comprises an amino acid sequence of SEQ ID NO: 15.

13. The bispecific antibody of claim 11, wherein the Fc domain region comprises an amino acid sequence of SEQ ID NO: 16.

14. The bispecific antibody of any one of claims 1-13, wherein the bispecific antibody is a human antibody, a humanized antibody or a chimeric antibody.

15. The bispecific antibody of any one of claims 1-14, wherein the IgG antibody is an IgG1 or an IgG4 antibody.

16. A nucleic acid sequence encoding any one of claims 1-15.

17. A multispecific antibody comprising the antigen binding regions of the bispecific antibody of any one of claims 1-15.

18. A method of treating, preventing, or delaying the progression of pathologies associated with aberrant CD38 expression or activity in a subject in need thereof, comprising administering an effective amount of the bispecific antibody of any one of claims 1-15 or the multispecific antibody of claim 17.

19. The method of claim 18, wherein the pathology is cancer.

20. The method of claim 19, wherein the cancer is a multiple myeloma.

21. The method of claim 19, wherein the cancer is lymphoma.

22. The method of claim 23, wherein the cancer is leukemia.

23. A method of redirecting a NK cell response in a subject in need thereof, comprising administering an effective amount of the bispecific antibody of any one of claims 1-15 or the multispecific antibody of claim 17.

24. The method of claim 23, wherein the NK cell response is NK-mediated cytotoxicity or antibody-dependent cellular cytotoxicity (ADCC).

25. A method of promoting specific lysis of cells expressing CD38 (CD38+ cells) by natural killer (NK) cells, comprising contacting the CD38+ cells with an effective amount of the bispecific antibody according to any one of claims 1-15,
wherein the effective amount is an amount sufficient to promote the specific lysis of the CD38+ cells by NK cells.

26. The method of claim 25, wherein the CD38+ cells are multiple myeloma cells or lymphoma cells.

27. The method of claim 25, wherein the contacting step comprises administering the bispecific antibody to a subject suffering from or a risk for multiple myeloma or lymphoma.

28. A method of inhibiting proliferation of multiple myeloma cells, lymphoma cells or CD38+ cancer cells in a subject treated with a bispecific antibody according to any one of claims 1-15, comprising administering an effective amount of natural killer (NK) cell.

29. The method of any one of claims 18-24, wherein the method comprises administering an effective amount of natural killer (NK) cells.

30. A combination therapy or a kit for treatment of multiple myeloma, lymphoma or a CD38+ cancer, comprising NK cells and a bispecific antibody according to any one of claims 1-15.

31. The bispecific antibody according to any one of claims 1-15 for use in combination with NK cells.

31. Use of natural killer (NK) cells and a bispecific antibody according to any one of claims 1-15 for treatment of multiple myeloma, a leukemia (e.g., acute myeloid leukemia), lymphoma or a CD38+ cancer.

32. A kit comprising a bispecific antibody of any one of claims 1-15.

33. The method of any one of claims 25-29, wherein the NK cells are induced pluripotent stem cell-derived natural killer (iPSC-NK) cells.

34. The method of any one of claims 25-29, wherein the NK cells are donor-derived NK cells.

35. The method of any one of claims 25-29, wherein, the NK cells are irradiated immortalized NK cells.

36. The method of any one of claims 25-29, wherein the CD38+ cancer is a hematological malignancy or a solid tumor.
